(19) United States
(12) Patent Application Publication
(10) Pub. No.: US 2023/0002746 A1
(43) Pub. Date: Jan. 5, 2023

(54) BASE-EDITING SYSTEMS

(71) Applicant: Inari Agriculture Technology, Inc., Cambridge, MA (US)

(72) Inventors: Adam Patrick JOYCE, Cambridge, MA (US); John Paul ISSA, Cambridge, MA (US); Michael Andreas KOCK, Cambridge, MA (US)

(73) Assignee: Inari Agriculture Technology, Inc., Cambridge, MA (US)

(21) Appl. No.: 17/772,106

(22) PCT Filed: Oct. 20, 2020
(86) PCT No.: PCT/US2020/058186

§ 371 Date: Apr. 26, 2022

Related U.S. Application Data
(60) Provisional application No. 62/929,007, filed on Oct. 31, 2019.

(51) Int. Cl. C12N 9/22; C12N 9/78; C12N 15/11; A61K 47/64; A61K 47/54; C12N 15/90; A61K 38/46; A61K 38/50; A61K 31/7105; C12N 15/82

(52) U.S. Cl. CPC C12N 9/22; C12N 9/78; C12Y 305/04005; C12N 15/11; A61K 47/6455; A61K 47/545; C12N 15/907; A61K 38/465; A61K 38/50; A61K 31/7105; C12N 15/8213; C12N 2800/80; C07K 2319/80; C12N 2310/20; C07K 2319/81

(57) ABSTRACT

The present disclosure relates to base-editing systems including a fusion protein including a DNA-binding domain and a cytidine deaminase domain and a non-protein uracil-DNA glycosylase inhibitor, and methods of using the same. The DNA-binding domains of base-editing systems of the present disclosure include domains with a variety of target region possibilities, which increase the number and type of sequences that can be edited. The npUGIs of the base-editing systems of the present disclosure improve UDG inhibition (e.g., UDG inhibition is more complete) and are suitable for use in a wide range of organisms.

### CROSS-REFERENCE TO RELATED APPLICATIONS

This application claims priority to U.S. Provisional Application No. 62/929,007, filed on Oct. 31, 2019, the content of which is hereby incorporated by reference in its entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 165362000340seglist.txt, date recorded: Oct. 19, 2020, size: 3,827 KB).

### FIELD

The present disclosure relates to base-editing systems including a fusion protein including a DNA-binding domain and a cytidine deaminase domain and a non-protein uracil-DNA glycosylase inhibitor, and methods of using the same.

### BACKGROUND

Targeted editing of nucleic acid sequences, for example, the targeted cleavage or the targeted introduction of a specific modification into genomic DNA, is a highly promising approach for the study of gene function and also has the potential to provide new therapies for human genetic diseases (Humbert et al., Crit Rev Biochem Mol (2012) 47(3):264-81. PMID: 22530743). Many genetic disorders have been identified as having specific nucleotide changes underlying the disorder (for example, a C to T change in a specific codon of a gene associated with a disease; Cargill et al., Nat Genet (1999) 22(3):231-8. PMID: 10391209). In addition, targeted editing can be used for a wide range of applications in plants, including modifying sequences to add or improve desirable characteristics. An ideal nucleic acid editing technology possesses three characteristics: (1) high efficiency of installing the desired modification; (2) minimal off-target activity; and (3) the ability to be programmed to precisely edit any site in a given nucleic acid. Therefore, precision gene editing would provide an effective tool to precisely modify proteins and would represent a powerful tool suitable for a wide range of applications in medical, industrial, agronomic, and research fields.

There are a variety of current genome engineering tools that are able to cleave DNA of a particular sequence, including engineered zinc finger nucleases (ZFNs; Urnov et al., Nat Rev Genet (2010) 11(9):636-46. PMID: 20717154), transcription activator-like effector nucleases (TALENs) (Joung and Sander, Nat Rev Mol Cell Biol (2013) 14(1):49-55. PMID: 23169466), and the RNA-guided DNA endonuclease Cas9 (Charpentier and Doudna, Nature (2013) 495, (7439):50-1. PMID: 23467164). These existing tools can be used to mutate the DNA at the cleavage site via non-homologous end joining (NHEJ) or to replace the DNA surrounding the cleavage site via homology-directed repair (HDR) (Pan et al., Mol Biotechnol (2013) 55(1):54-62. PMID: 23089945). NHEJ and HDR are both, however, stochastic processes that typically result in modest gene editing efficiencies as well as off-target gene alterations (Santiago et al., PNAS (2008) 105(15):5809-14. PMID: 18359850).

Because of this limited efficiency, research has focused on developing composite genome engineering tools. These composite tools include additional components, such as uracil-DNA glycosylase inhibitors, to increase gene editing efficiency. In developing these systems, however, it has been found that Cas9 base-editing systems are limited in their ability to target some regions of the genome because Cas9 is highly dependent on the presence of a nearby protospacer adjacent motif (PAM). Further, base-editing systems that have protein uracil-DNA glycosylase inhibitors suffer from incomplete uracil-DNA glycosylase (UDG) inhibition, which decreases base-editing efficiency. Last, there exists the possibility that some organisms possess glycosylase variants that bind poorly to protein uracil-DNA glycosylase inhibitors, and thus provide incomplete protection of deaminated cytosine bases.

There exists therefore a need for genome engineering tools able to edit a target nucleotide sequence at higher efficiency with a reduced amount of off-target alterations as compared to the current genome engineering tools. These tools would address a need for efficient and reliable precision genome editing in medical, industrial, agronomic, and research fields.

### BRIEF SUMMARY

In order to meet these needs, the present disclosure is directed to base-editing systems including a fusion protein including a DNA-binding domain and a cytidine deaminase domain and a non-protein uracil-DNA glycosylase inhibitor (npUGI). The DNA-binding domains of base-editing systems of the present disclosure include domains with a variety of target region possibilities, which increase the number and type of sequences that can be edited. The npUGIs of the base-editing systems of the present disclosure improve UDG inhibition (e.g., UDG inhibition is more complete) and are suitable for use in a wide range of organisms. The base-editing systems of the present disclosure are able to specifically edit target nucleotide sequences at higher efficiency.

In some aspects, the present disclosure relates to a base-editing system including (i) a fusion protein including (a) a DNA-binding domain and (b) a cytidine deaminase domain; and (ii) a non-protein uracil-DNA glycosylase inhibitor (npUGI). In some embodiments that may be combined with any of the preceding embodiments, the DNA-binding domain is selected from the group of a Cas domain, a Transcription Activator-Like Effector (TALE) domain, or a Zinc finger (ZF) domain. In some embodiments that may be combined with any of the preceding embodiments, the Cas domain is selected from the group of a Cas 9 domain, a Cas12a domain, a Cas12h domain, a Cas12i domain, a CasX domain, a CasY domain, a C2c1 domain, a C2c2 domain, or a C2c3 domain. In some embodiments that may be combined with any of the preceding embodiments, the Cas domain is the Cas12a domain. In some embodiments that may be combined with any of the preceding embodiments, the Cas12a domain includes an amino acid sequence with at least 85% sequence identity, at least 90% sequence identity, at least 95% sequence identity, or at least 99% sequence identity to SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413. In some embodiments that may be combined with any of the preceding embodiments, the Cas12a domain is SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413. In some embodiments that may be combined with any of the preceding embodiments, the Cas domain is the Cas9 domain. In some embodiments that may be combined with any of the preceding embodiments, the Cas9 domain includes an amino acid sequence with at least 85% sequence identity, at least 90% sequence identity, at least 95% sequence identity, or at least 99% sequence identity to SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 325, SEQ ID NO: 674, SEQ ID NO: 1869, SEQ ID NO: 1870, SEQ ID NO: 1871, SEQ ID NO: 1872, SEQ ID NO: 1873, SEQ ID NO: 1874, SEQ ID NO: 1875, or SEQ ID NO: 1876. In some embodiments, the Cas9 domain includes SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 325, SEQ ID NO: 674, SEQ ID NO: 1869, SEQ ID NO: 1870, SEQ ID NO: 1871, SEQ ID NO: 1872, SEQ ID NO: 1873, SEQ ID NO: 1874, SEQ ID NO: 1875, or SEQ ID NO: 1876. In some embodiments that may be combined with any of the preceding embodiments, the Cas domain is selected from the group of a Cas nickase domain or a deactivated Cas domain. In some embodiments that may be combined with any of the preceding embodiments, the base-editing system further includes a guide RNA, the guide RNA has a length in the range of 15-100 nucleotides, and the guide RNA includes a sequence of at least 10 contiguous nucleotides that is complementary to a target sequence. In some embodiments that may be combined with any of the preceding embodiments, the DNA-binding domain is the TALE domain. In some embodiments, the DNA-binding domain is the ZF domain.

In some embodiments that may be combined with any of the preceding embodiments, the cytidine deaminase domain is an apolipoprotein B mRNA-editing complex (APOBEC) family deaminase domain. In some embodiments that may be combined with any of the preceding embodiments, the APOBEC family deaminase domain is selected from the group of an APOBEC1 deaminase domain, an APOBEC2 deaminase domain, an APOBEC3A deaminase domain, an APOBEC3B deaminase domain, an APOBEC3C deaminase domain, an APOBEC3D deaminase domain, an APOBEC3F deaminase domain, an APOBEC3G deaminase domain, an APOBEC3H deaminase domain, or an APOBEC4 deaminase domain. In some embodiments that may be combined with any of the preceding embodiments, the APOBEC family deaminase domain includes an amino acid sequence with at least 85% sequence identity, at least 90% sequence identity, at least 95% sequence identity, or at least 99% sequence identity to SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain includes SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain is an activation-induced deaminase (AID) domain.

In some embodiments that may be combined with any of the preceding embodiments, the npUGI is selected from the group of a small molecule inhibitor of uracil-DNA glycosylase (UDG) or a nucleic acid inhibitor of UDG. In some embodiments that may be combined with any of the preceding embodiments, the npUGI is the small molecule inhibitor of UDG. In some embodiments that may be combined with any of the preceding embodiments, the small molecule inhibitor of UDG is a compound of formula (I):

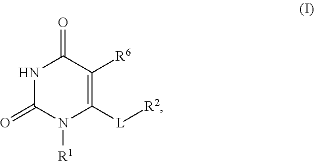

or a pharmaceutically acceptable salt thereof, wherein: R¹ is H, a furanose carbohydrate, a pyranose carbohydrate, a carbohydrate mimetic, C₁₋₁₆alkyl, C₁₋₁₆alkenyl, C₁₋₁₆alkynyl, C₁₋₁₆alkoxy, or C₆₋₂₀aryl, wherein the furanose carbohydrate, or a derivative thereof, pyranose carbohydrate, or a derivative thereof, carbohydrate mimetic, C₁₋₁₆alkyl, C₁₋₁₆alkenyl, C₁₋₁₆alkynyl, C₁₋₁₆alkoxy, or C₆₋₂₀aryl is independently optionally substituted with one or more substituents selected from the group of hydroxyl, halo, cyano, NO₂, N(R⁴)(R⁵), C₁₋₁₆alkoxy, or C₆₋₂₀aryl, wherein the C₆₋₂₀aryl is further optionally substituted with one or more substituents selected from the group of C₁₋₁₆alkyl, C₁₋₁₆alkenyl, C₁₋₁₆alkynyl, C₁₋₁₆alkoxy, hydroxyl, halo, cyano, NO₂, or N(R⁴)(R⁵); L is O, S, or *—N(R³)—**, wherein R³ is H or C₁₋₁₆alkyl, and ** indicates the point of attachment to the R² moiety and * indicates the point of attachment to the remainder of the molecule; R² is H, N(R⁴)(R⁵), or C₆₋₂₀aryl, wherein the C₆₋₂₀aryl is independently substituted with one or more substituents selected from the group of C₁₋₁₆alkyl, C₁₋₁₆alkenyl, C₁₋₁₆alkynyl, or C₁₋₁₆alkoxy; R⁴ and R⁵ are each independently H or C₆₋₂₀aryl; and R⁶ is H or halo. In some embodiments that may be combined with any of the preceding embodiments, R¹ is H. In some embodiments that may be combined with any of the preceding embodiments, R¹ is C₁₋₁₆alkyl, wherein the C₁₋₁₆alkyl is optionally substituted with one or more substituents selected from the group of hydroxyl, halo, cyano, NO₂, N(R⁴)(R⁵), C₁₋₁₆alkoxy, or C₆₋₂₀aryl, wherein the C₆₋₂₀aryl is further optionally substituted with one or more substituents selected from the group of C₁₋₁₆alkyl, C₁₋₁₆alkenyl, C₁₋₁₆alkynyl, C₁₋₁₆alkoxy, hydroxyl, halo, cyano, NO₂, or N(R⁴)(R⁵), wherein R⁴ and R⁵ are each independently H or C₆₋₂₀aryl. In some embodiments that may be combined with any of the preceding embodiments, L is NR³, wherein R³ is H. In some embodiments that may be combined with any of the preceding embodiments, L is O. In some embodiments that may be combined with any of the preceding embodiments, R² is C₆₋₂₀aryl, wherein the C₆₋₂₀aryl is independently substituted with one or more substituents selected from the group of C₁₋₁₆alkyl, C₁₋₁₆alkenyl, C₁₋₁₆alkynyl, or C₁₋₁₆alkoxy. In some embodiments that may be combined with any of the preceding embodiments, R⁶ is H. In some embodiments that may be combined with any of the preceding embodiments, R⁶ is halo. In some embodiments that may be combined with any of the preceding embodiments, the base-editing system further includes a pharmaceutically acceptable carrier, diluent, or excipient. In some embodiments that may be combined with any of the preceding embodiments, the compound of formula (I) is incorporated into a macromolecule. In some embodiments that may be combined with any of the preceding embodiments, the macromolecule is an oligonucleotide. In some embodiments that may be combined with any of the preceding embodiments, the npUGI is the nucleic acid inhibitor of UDG. In some embodiments that may be combined with any of the preceding embodiments, the nucleic acid inhibitor of UDG is selected from the group of a double-stranded RNA inhibitor, an antisense oligonucleotide inhibitor, a RNA/DNA hybrid inhibitor, a microRNA inhibitor, or a siRNA inhibitor. In some embodiments that may be combined with any of the preceding embodiments, the nucleic acid inhibitor reversibly inhibits an activity of an UDG. In some embodiments that may be combined with any of the preceding embodiments, the Cas domain is the Cas nickase domain or the deactivated Cas domain, the npUGI is a RNA guide of the Cas nickase domain or the deactivated Cas domain, the RNA guide includes a 5′ end and a 3′ end, and the RNA guide includes one or more deoxyuracil (dU) bases added to the 5′ end or the 3′ end. In some embodiments that may be combined with any of the preceding embodiments, the one or more dU bases are modified to resist glycosidic bond cleavage, have enhanced in vivo stability, or have enhanced ability to bind UDG, as compared to unmodified dU bases. In some embodiments that may be combined with any of the preceding embodiments, the one or more dU bases include a section of double-stranded DNA. In some embodiments that may be combined with any of the preceding embodiments, the UDG is an animal UDG. In some embodiments that may be combined with any of the preceding embodiments, the UDG is a plant UDG. In some embodiments that may be combined with any of the preceding embodiments, the plant UDG includes an amino acid sequence with at least 85% sequence identity, at least 90% sequence identity, at least 95% sequence identity, or at least 99% sequence identity to SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments that may be combined with any of the preceding embodiments, the plant UDG includes SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments that may be combined with any of the preceding embodiments, the fusion protein and the npUGI are chemically linked. In some embodiments that may be combined with any of the preceding embodiments, the fusion protein and the npUGI are not chemically linked.

In some aspects, the present disclosure relates to a base-editing system including (i) a fusion protein including (a) a Cas9 nickase domain or a dCas9 domain and (b) an APOBEC family deaminase domain; and (ii) a npUGI.

In some aspects, the present disclosure relates to a base-editing system including (i) a fusion protein including (a) a Cas12a nickase domain or a dCas12a domain and (b) an APOBEC family deaminase domain; and (ii) a npUGI.

In some aspects, the present disclosure relates to a base-editing system including (i) a fusion protein including (a) a TALE domain and (b) an APOBEC family deaminase domain; and (ii) a npUGI.

In some aspects, the present disclosure relates to a base-editing system including (i) a fusion protein including (a) a ZF domain and (b) an APOBEC family deaminase domain; and (ii) a npUGI.

In some embodiments that may be combined with any of the preceding embodiments, the npUGI is selected from the group of a small molecule inhibitor of uracil-DNA glycosylase (UDG) or a nucleic acid inhibitor of UDG. In some embodiments that may be combined with any of the preceding embodiments, the npUGI is a RNA guide of the Cas9 nickase domain, the dCas9 domain, the Cas12a nickase domain, or the dCas12a domain, wherein the RNA guide includes a 5′ end and a 3′ end, and wherein the RNA guide includes one or more deoxyuracil (dU) bases added to the 5′ end or the 3′ end.

In some aspects, the present disclosure relates to methods of editing a target nucleic acid molecule, including contacting a target nucleic acid molecule with the base-editing system of any of the preceding embodiments, wherein the DNA binding domain of the fusion protein is the Cas domain.

In some aspects, the present disclosure relates to methods of editing a target nucleic acid molecule, including contacting a target nucleic acid molecule with the base-editing system of any of the preceding embodiments, wherein the DNA binding domain of the fusion protein is the TALE domain or the ZF domain.

In some embodiments that may be combined with any of the preceding embodiments, the fusion protein and the npUGI of the base-editing system are chemically linked. In some embodiments that may be combined with any of the preceding embodiments, the fusion protein and the npUGI are chemically linked by in vitro complexing or in vivo complexing. In some embodiments that may be combined with any of the preceding embodiments, the fusion protein and the npUGI of the base-editing system are not chemically linked. In some embodiments that may be combined with any of the preceding embodiments, the fusion protein and the npUGI are co-delivered, the fusion protein is delivered before the npUGI, or the npUGI is delivered before the fusion protein. In some embodiments that may be combined with any of the preceding embodiments, the target nucleic acid sequence is a double-stranded DNA (dsDNA) sequence, and the target nucleic acid sequence is in the genome of an organism. In some embodiments that may be combined with any of the preceding embodiments, the target nucleic acid sequence is associated with a disease or disorder, and the target nucleic acid sequence includes a point mutation. In some embodiments that may be combined with any of the preceding embodiments, the point mutation is a T to C point mutation, and the T to C point mutation is associated with the disease or disorder. In some embodiments that may be combined with any of the preceding embodiments, the point mutation is a T to C point mutation in a codon of the target nucleic acid sequence, and the point mutation results in an amino acid change in a polypeptide encoded by the target nucleic acid sequence as compared to a wild-type polypeptide encoded by a wild-type nucleic acid sequence without the point mutation. In some embodiments that may be combined with any of the preceding embodiments, contacting the target nucleic acid sequence with the base-editing system produces a nucleic acid sequence that is not associated with the disease or disorder. In some embodiments that may be combined with any of the preceding embodiments, contacting the target nucleic acid sequence with the base-editing system produces a wild-type nucleic acid sequence without the point mutation. In some embodiments that may be combined with any of the preceding embodiments, the disease or disorder is cystic fibrosis, phenylketonuria, epidermolytic hyperkeratosis (EHK), Charcot-Marie-Toot disease type 4J, neuroblastoma (NB), von Willebrand disease (vWD), myotonia congenital, hereditary renal amyloidosis, dilated cardiomyopathy (DCM), hereditary lymphedema, familial Alzheimer's disease, HIV, Prion disease, chronic infantile neurologic cutaneous articular syndrome (CINCA), desmin-related myopathy (DRM), a neoplastic disease associated with a mutant PI3KCA protein, a mutant CTNNBl protein, a mutant HRAS protein, or a mutant p53 protein. In some embodiments that may be combined with any of the preceding embodiments, the contacting is performed in vivo. In some embodiments that may be combined with any of the preceding embodiments, the contacting is performed in vitro. In some embodiments that may be combined with any of the preceding embodiments, the organism is a mammal, and the mammal is selected from the group of mouse, rat, human, pig, cow, chicken, rhesus monkey, or guinea pig. In some embodiments that may be combined with any of the preceding embodiments, the target nucleic acid sequence is selected from the group of a target nucleic acid sequence associated with a disease, a target nucleic acid sequence associated with a disorder, a target nucleic acid sequence associated with metabolic function, a target nucleic acid sequence associated with reproductive function, a target nucleic acid sequence associated with disease resistance function, a target nucleic acid sequence associated with stress tolerance function, a target nucleic acid sequence associated with agronomic function, or a target nucleic acid sequence associated with nutritional function. In some embodiments that may be combined with any of the preceding embodiments, contacting the target nucleic acid sequence with the base-editing system produces a nucleic acid sequence selected from the group of a nucleic acid sequence with a corrected deleterious mutation, a nucleic acid sequence derivative, a modified nucleic acid sequence, a nucleic acid sequence with an inserted sequence, a nucleic acid sequence with improved function, or a nucleic acid sequence with altered function. In some embodiments that may be combined with any of the preceding embodiments, the target nucleic acid sequence includes a point mutation, and contacting the target nucleic acid sequence with the base-editing system produces a wild-type nucleic acid sequence without the point mutation. In some embodiments that may be combined with any of the preceding embodiments, the contacting is performed in vivo. In some embodiments that may be combined with any of the preceding embodiments, the contacting is performed in vitro. In some embodiments that may be combined with any of the preceding embodiments, the organism is a plant, and the plant is selected from the group of sorghum, corn, tomato, rice, soybean, or wheat.

In some aspects, the present disclosure relates to methods for in vivo editing of a cytosine residue of a target DNA sequence, the method including a) contacting the target DNA sequence with the base-editing system of any one of claims 2-11 and 14-41, wherein the DNA binding domain of the fusion protein is the Cas domain, and a guide nucleic acid; and b) cultivating the cell through a cell division.

In some aspects, the present disclosure relates to methods for editing a nucleobase pair of a double-stranded DNA sequence, the method including: a) contacting a target region of the double-stranded DNA sequence, wherein the target region includes a target nucleobase pair, with the base-editing system of any one of claims 2-11 and 14-41, wherein the DNA binding domain of the fusion protein is the Cas domain, and a guide nucleic acid, wherein the target region includes a target nucleobase pair; b) inducing strand separation of said target region; c) converting a first nucleobase of said target nucleobase pair in a single strand of the target region to a second nucleobase; and d) cutting no more than one strand of said target region; wherein a third nucleobase complementary to the first nucleobase base is replaced by a fourth nucleobase complementary to the second nucleobase and the method causes less than 20% indel formation in the double-stranded DNA sequence. In some embodiments that may be combined with any of the preceding embodiments, the cut single strand is hybridized to the guide nucleic acid. In some embodiments that may be combined with any of the preceding embodiments, the cut single strand is opposite to the strand including the first nucleobase. In some embodiments that may be combined with any of the preceding embodiments, the first base is a cytosine. In some embodiments that may be combined with any of the preceding embodiments, the second base is a uracil. In some embodiments that may be combined with any of the preceding embodiments, the Cas domain is selected from the group of a Cas nickase domain or a deactivated Cas domain.

In some aspects, the present disclosure relates to methods for editing a nucleobase pair of a double-stranded DNA sequence, the method including: a) contacting a target region of the double-stranded DNA sequence, wherein the target region includes a target nucleobase pair, with the base-editing system of any of claims 2-11 and 14-41, wherein the DNA binding domain of the fusion protein is the Cas domain, and a guide nucleic acid; b) inducing strand separation of said target region; c) converting a first nucleobase of said target nucleobase pair in a single strand of the target region to a second nucleobase; d) cutting no more than one strand of said target region; wherein a third nucleobase complementary to the first nucleobase base is replaced by a fourth nucleobase complementary to the second nucleobase; and e) replacing the second nucleobase with a fifth nucleobase that is complementary to the fourth nucleobase, thereby generating an intended edited basepair, wherein the efficiency of generating the intended edited basepair is at least 5%. In some embodiments that may be combined with any of the preceding embodiments, the cut single strand is hybridized to the guide nucleic acid. In some embodiments that may be combined with any of the preceding embodiments, the cut single strand is opposite to the strand including the first nucleobase. In some embodiments that may be combined with any of the preceding embodiments, the first base is a cytosine. In some embodiments that may be combined with any of the preceding embodiments, the second base is an uracil. In some embodiments that may be combined with any of the preceding embodiments, the Cas domain is selected from the group of a Cas nickase domain or a deactivated Cas domain.

In some embodiments that may be combined with any of the preceding embodiments, the Cas domain is selected from the group of a Cas 9 domain, a Cas12a domain, a Cas12h domain, a Cas12i domain, a CasX domain, a CasY domain, a C2c1 domain, a C2c2 domain, or a C2c3 domain. In some embodiments that may be combined with any of the preceding embodiments, the Cas domain is the Cas12a domain, and the Cas12a domain includes an amino acid sequence with at least 85% sequence identity, at least 90% sequence identity, at least 95% sequence identity, or at least 99% sequence identity to SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413. In some embodiments that may be combined with any of the preceding embodiments, the Cas domain is the Cas9 domain, and the Cas9 domain includes an amino acid sequence with at least 85% sequence identity, at least 90% sequence identity, at least 95% sequence identity, or at least 99% sequence identity to SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 325, SEQ ID NO: 674, SEQ ID NO: 1869, SEQ ID NO: 1870, SEQ ID NO: 1871, SEQ ID NO: 1872, SEQ ID NO: 1873, SEQ ID NO: 1874, SEQ ID NO: 1875, or SEQ ID NO: 1876.

In some aspects, the present disclosure relates to methods for in vivo editing of a cytosine residue of a target DNA sequence, the method including a) contacting the target DNA sequence with the base-editing system of any one of claims 12-41, wherein the DNA binding domain of the fusion protein is the TALE domain or the ZF domain; and b) cultivating the cell through a cell division.

In some embodiments that may be combined with any of the preceding embodiments, the cytidine deaminase domain is an APOBEC family deaminase domain. In some embodiments that may be combined with any of the preceding embodiments, the APOBEC family deaminase domain includes an amino acid sequence with at least 85% sequence identity, at least 90% sequence identity, at least 95% sequence identity, or at least 99% sequence identity to SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the npUGI of the base-editing system is selected from the group of a small molecule inhibitor of uracil-DNA glycosylase (UDG) or a nucleic acid inhibitor of UDG. In some embodiments that may be combined with any of the preceding embodiments, the Cas domain is the Cas nickase domain or the deactivated Cas domain, the npUGI of the base-editing system is a RNA guide of the Cas nickase domain or the deactivated Cas domain, the RNA guide has a 5′ end and a 3′ end, and the RNA guide has one or more deoxyuracil (dU) bases added to the 5′ end or the 3′ end. In some embodiments that may be combined with any of the preceding embodiments, the fusion protein and the npUGI are chemically linked. In some embodiments that may be combined with any of the preceding embodiments, the fusion protein and the npUGI are chemically linked by in vitro complexing or in vivo complexing. In some embodiments that may be combined with any of the preceding embodiments, the fusion protein and the npUGI are not chemically linked. In some embodiments that may be combined with any of the preceding embodiments, the fusion protein and the npUGI are co-delivered, the fusion protein is delivered before the npUGI, or the npUGI is delivered before the fusion protein. In some embodiments that may be combined with any of the preceding embodiments, the DNA sequence is in the genome of an organism. In some embodiments that may be combined with any of the preceding embodiments, the organism is a mammal selected from the group of mouse, rat, human, pig, cow, chicken, rhesus monkey, or guinea pig. In some embodiments that may be combined with any of the preceding embodiments, the organism is a plant selected from the group of sorghum, corn, tomato, rice, soybean, or wheat.

The description of exemplary embodiments of the base-editing systems above is provided for illustration purposes only and not meant to be limiting. Additional base-editing systems, e.g., variations of the exemplary systems described in detail above, are also embraced by this disclosure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1 shows a diagram of a base-editing system inhibiting DNA glycosylase excision. A dCas DNA binding domain (large light grey oval, labelled “dCas”) linked to a deaminase domain (small light grey oval, labelled “Deaminase”) is shown bound to a nucleic acid (black boxes with dashed line through middle) near a PAM sequence (box labelled “PAM”). A crRNA with a poly deoxy-uracil (“dU”) extension (black line with “dU”s at end) is complexed with the dCas DNA binding domain. The crRNA dU extension provides a secondary substrate for DNA glycosylase (grey oval, labelled “DNA Glycosylase”; dU substrate shown as white circle within grey oval), thus reducing the likelihood that the exposed edited base (half-box to the left of PAM sequence) will be bound and excised by DNA glycosylase before being encoded by mismatch repair or replication machinery.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

Some aspects of this disclosure provide a base-editing system that includes a domain capable of binding to a nucleotide sequence, for example, a DNA binding domain, (e.g., a Cas9 domain, a Cas12a domain, a Cas12h domain, a Cas12i domain, a CasX domain, a CasY domain, a C2c1 domain, a C2c2 domain, a C2c3 domain, a TALE domain, or a ZF domain), a DNA-editing domain (e.g., a deaminase domain), and a non-protein uracil-DNA glycosylase inhibitor (e.g., an organochemical compound, a nucleotide-based inhibitor, etc.). The deamination of a nucleobase by a deaminase can lead to a point mutation at the respective residue, which is referred to herein as nucleic acid editing. A base-editing system including a DNA binding domain, a DNA editing domain, and a non-protein uracil-DNA glycosylase inhibitor can thus be used for the targeted editing of nucleic acid sequences. Such base-editing systems are useful for targeted editing of DNA in vitro (e.g., for the generation of mutant cells, animals, or plants); for the introduction of targeted mutations (e.g., for the correction of genetic defects in cells ex vivo, such as in cells obtained from a subject that are subsequently re-introduced into the same or another subject); and for the introduction of targeted mutations (e.g., the correction of genetic defects or the introduction of deactivating mutations in disease-associated genes in a subject). Typically, the DNA binding domain of the base-editing systems described herein is a Cas9 domain, a Cas12a domain, a Cas12h domain, a Cas12i domain, a CasX domain, a CasY domain, a C2c1 domain, a C2c2 domain, a C2c3 domain, a TALE domain, or a ZF domain. Methods for the use of the base-editing systems as described herein are also provided.

### Base-Editing Systems

In some aspects, the present disclosure relates to a base-editing system including (i) a fusion protein including (a) a DNA-binding domain and (b) a cytidine deaminase domain; and (ii) a non-protein uracil-DNA glycosylase inhibitor (npUGI). In some embodiments that may be combined with any of the preceding embodiments, the DNA-binding domain is selected from the group of a Cas domain, a Transcription Activator-Like Effector (TALE) domain, or a Zinc finger (ZF) domain. In some embodiments that may be combined with any of the preceding embodiments, the Cas domain is selected from the group of a Cas 9 domain, a Cas12a domain, a Cas12h domain, a Cas12i domain, a CasX domain, a CasY domain, a C2c1 domain, a C2c2 domain, or a C2c3 domain. In some embodiments that may be combined with any of the preceding embodiments, the Cas domain is the Cas12a domain. In some embodiments that may be combined with any of the preceding embodiments, the Cas12a domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413. In some embodiments that may be combined with any of the preceding embodiments, the Cas12a domain is SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413. In some embodiments that may be combined with any of the preceding embodiments, the Cas domain is the Cas9 domain. In some embodiments that may be combined with any of the preceding embodiments, the Cas9 domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 325, SEQ ID NO: 674, SEQ ID NO: 1869, SEQ ID NO: 1870, SEQ ID NO: 1871, SEQ ID NO: 1872, SEQ ID NO: 1873, SEQ ID NO: 1874, SEQ ID NO: 1875, or SEQ ID NO: 1876. In some embodiments that may be combined with any of the preceding embodiments, the Cas9 domain includes SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 325, SEQ ID NO: 674, SEQ ID NO: 1869, SEQ ID NO: 1870, SEQ ID NO: 1871, SEQ ID NO: 1872, SEQ ID NO: 1873, SEQ ID NO: 1874, SEQ ID NO: 1875, or SEQ ID NO: 1876. In some embodiments that may be combined with any of the preceding embodiments, the Cas9 domain is SEQ ID NO: 674. In some embodiments that may be combined with any of the preceding embodiments, the Cas domain is the C2c1 domain, and the C2c1 domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 367. In some embodiments that may be combined with any of the preceding embodiments, the C2c1 domain is SEQ ID NO: 367. In some embodiments that may be combined with any of the preceding embodiments, the Cas domain is the C2c2 domain, and the C2c2 domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 368. In some embodiments that may be combined with any of the preceding embodiments, the C2c2 domain is SEQ ID NO: 368. In some embodiments that may be combined with any of the preceding embodiments, the Cas domain is the C2c3 domain, and the C2c3 domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 369. In some embodiments that may be combined with any of the preceding embodiments, the C2c3 domain is SEQ ID NO: 369. In some embodiments that may be combined with any of the preceding embodiments, the Cas domain is the CasX domain, and the CasX domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 370 or SEQ ID NO: 371. In some embodiments that may be combined with any of the preceding embodiments, the CasX domain is SEQ ID NO: 370 or SEQ ID NO: 371. In some embodiments that may be combined with any of the preceding embodiments, the Cas domain is the CasY domain, and the CasY domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 372. In some embodiments that may be combined with any of the preceding embodiments, the CasY domain is SEQ ID NO: 372. In some embodiments that may be combined with any of the preceding embodiments, the DNA-binding domain is selected from the group of an Argonaute domain or a meganuclease domain. In some embodiments that may be combined with any of the preceding embodiments, the DNA-binding domain is an Argonaute domain, and the Argonaute domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 366. In some embodiments that may be combined with any of the preceding embodiments, the Argonaute domain is SEQ ID NO: 366. In some embodiments that may be combined with any of the preceding embodiments, the Cas domain is selected from the group of a Cas nickase domain or a deactivated Cas domain. In some embodiments that may be combined with any of the preceding embodiments, the base-editing system further includes a guide RNA, the guide RNA has a length in the range of 15-100 nucleotides, and the guide RNA includes a sequence of at least 10 contiguous nucleotides that is complementary to a target sequence. In some embodiments that may be combined with any of the preceding embodiments, the DNA-binding domain is the TALE domain. In some embodiments that may be combined with any of the preceding embodiments, the DNA-binding domain is the ZF domain.

In some embodiments that may be combined with any of the preceding embodiments, the cytidine deaminase domain is an apolipoprotein B mRNA-editing complex (APOBEC) family deaminase domain. In some embodiments that may be combined with any of the preceding embodiments, the APOBEC family deaminase domain is selected from the group of an APOBEC1 deaminase domain, an APOBEC2 deaminase domain, an APOBEC3A deaminase domain, an APOBEC3B deaminase domain, an APOBEC3C deaminase domain, an APOBEC3D deaminase domain, an APOBEC3F deaminase domain, an APOBEC3G deaminase domain, an APOBEC3H deaminase domain, or an APOBEC4 deaminase domain. In some embodiments that may be combined with any of the preceding embodiments, the APOBEC family deaminase domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain includes SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain is an activation-induced deaminase (AID) domain. In some embodiments that may be combined with any of the preceding embodiments, the AID deaminase domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 266, SEQ ID NO: 267, SEQ ID NO: 268, SEQ ID NO: 269, SEQ ID NO: 607, SEQ ID NO: 608, or SEQ ID NO: 1879. In some embodiments that may be combined with any of the preceding embodiments, the AID deaminase domain is SEQ ID NO: 266, SEQ ID NO: 267, SEQ ID NO: 268, SEQ ID NO: 269, SEQ ID NO: 607, SEQ ID NO: 608, or SEQ ID NO: 1879.

In some embodiments that may be combined with any of the preceding embodiments, the npUGI is selected from the group of a small molecule inhibitor of uracil-DNA glycosylase (UDG) or a nucleic acid inhibitor of UDG. In some embodiments that may be combined with any of the preceding embodiments, the npUGI is the small molecule inhibitor of UDG. In some embodiments that may be combined with any of the preceding embodiments, the small molecule inhibitor of UDG is a compound of formula (I):

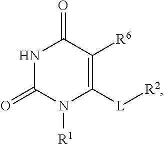

or a pharmaceutically acceptable salt thereof, wherein: R¹ is H, a furanose carbohydrate, a pyranose carbohydrate, a carbohydrate mimetic, C₁₋₁₆alkyl, C₁₋₁₆alkenyl, C₁₋₁₆alkynyl, C₁₋₁₆alkoxy, or C₆₋₂₀aryl, wherein the furanose carbohydrate, or a derivative thereof, pyranose carbohydrate, or a derivative thereof, carbohydrate mimetic, C₁₋₁₆alkyl, C₁₋₁₆alkenyl, C₁₋₁₆alkynyl, C₁₋₁₆alkoxy, or C₆₋₂₀aryl is independently optionally substituted with one or more substituents selected from the group of hydroxyl, halo, cyano, NO₂, N(R⁴)(R⁵), C₁₋₁₆alkoxy, or C₆₋₂₀aryl, wherein the C₆₋₂₀aryl is further optionally substituted with one or more substituents selected from the group of C₁₋₁₆alkyl, C₁₋₁₆alkenyl, C₁₋₁₆alkynyl, C₁₋₁₆alkoxy, hydroxyl, halo, cyano, NO₂, or N(R⁴)(R⁵); L is O, S, or *—N(R³)—**, wherein R³ is H or C₁₋₁₆alkyl, and ** indicates the point of attachment to the R² moiety and * indicates the point of attachment to the remainder of the molecule; R² is H, N(R⁴)(R⁵), or C₆₋₂₀aryl, wherein the C₆₋₂₀aryl is independently substituted with one or more substituents selected from the group of C₁₋₁₆alkyl, C₁₋₁₆alkenyl, C₁₋₁₆alkynyl, or C₁₋₁₆alkoxy; R⁴ and R⁵ are each independently H or C₆₋₂₀aryl; and R⁶ is H or halo. In some embodiments that may be combined with any of the preceding embodiments, R¹ is H. In some embodiments that may be combined with any of the preceding embodiments, R¹ is C₁₋₁₆alkyl, wherein the C₁₋₁₆alkyl is optionally substituted with one or more substituents selected from the group of hydroxyl, halo, cyano, NO₂, N(R⁴)(R⁵), C₁₋₁₆alkoxy, or C₆₋₂₀aryl, wherein the C₆₋₂₀aryl is further optionally substituted with one or more substituents selected from the group of C₁₋₁₆alkyl, C₁₋₁₆alkenyl, C₁₋₁₆alkynyl, C₁₋₁₆alkoxy, hydroxyl, halo, cyano, NO₂, or N(R⁴)(R⁵), wherein R₄ and R₅ are each independently H or C₆₋₂₀aryl. In some embodiments that may be combined with any of the preceding embodiments, L is NR³, wherein R³ is H. In some embodiments that may be combined with any of the preceding embodiments, L is O. In some embodiments that may be combined with any of the preceding embodiments, R² is C₆₋₂₀aryl, wherein the C₆₋₂₀aryl is independently substituted with one or more substituents selected from the group of C₁₋₁₆alkyl, C₁₋₁₆alkenyl, C₁₋₁₆alkynyl, or C₁₋₁₆alkoxy. In some embodiments that may be combined with any of the preceding embodiments, R⁶ is H. In some embodiments that may be combined with any of the preceding embodiments, R⁶ is halo. In some embodiments that may be combined with any of the preceding embodiments, the base-editing system further includes a pharmaceutically acceptable carrier, diluent, or excipient. In some embodiments that may be combined with any of the preceding embodiments, the compound of formula (I) is incorporated into a macromolecule. In some embodiments that may be combined with any of the preceding embodiments, the macromolecule is an oligonucleotide. In some embodiments that may be combined with any of the preceding embodiments, the npUGI is the nucleic acid inhibitor of UDG. In some embodiments that may be combined with any of the preceding embodiments, the nucleic acid inhibitor of UDG is selected from the group of a double-stranded RNA inhibitor, an antisense oligonucleotide inhibitor, a RNA/DNA hybrid inhibitor, a microRNA inhibitor, or a siRNA inhibitor. In some embodiments that may be combined with any of the preceding embodiments. In some embodiments that may be combined with any of the preceding embodiments, the Cas domain is the Cas nickase domain or the deactivated Cas domain, the npUGI is a RNA guide of the Cas nickase domain or the deactivated Cas domain, the RNA guide includes a 5′ end and a 3′ end, and the RNA guide includes one or more deoxyuracil (dU) bases added to the 5′ end or the 3′ end. In some embodiments that may be combined with any of the preceding embodiments, the one or more dU bases are modified to resist glycosidic bond cleavage, have enhanced in vivo stability, or have enhanced ability to bind UDG, as compared to unmodified dU bases. In some embodiments that may be combined with any of the preceding embodiments, the one or more dU bases include a section of double-stranded DNA. In some embodiments that may be combined with any of the preceding embodiments, the section of double-stranded DNA includes one or more U:T pairs, U:G pairs, U: A pairs, U: C pairs, dU:T pairs, dU:G pairs, dU: A pairs, dU: T pairs, or any combination thereof (e.g., any combination of U or dU base mismatch pairs). In some embodiments that may be combined with any of the preceding embodiments, the UDG is an animal UDG. In some embodiments that may be combined with any of the preceding embodiments, the UDG is a plant UDG. In some embodiments that may be combined with any of the preceding embodiments, the plant UDG includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments that may be combined with any of the preceding embodiments, the plant UDG includes SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments that may be combined with any of the preceding embodiments, the fusion protein and the npUGI are chemically linked. In some embodiments that may be combined with any of the preceding embodiments, the fusion protein and the npUGI are not chemically linked.

In some aspects, the present disclosure relates to a base-editing system including (i) a fusion protein including (a)) a Cas9 nickase domain or a dCas9 domain and (b) an APOBEC family deaminase domain; and (ii) a npUGI. In some embodiments that may be combined with any of the preceding embodiments, the APOBEC family deaminase domain is selected from the group of an APOBEC1 deaminase domain, an APOBEC2 deaminase domain, an APOBEC3A deaminase domain, an APOBEC3B deaminase domain, an APOBEC3C deaminase domain, an APOBEC3D deaminase domain, an APOBEC3F deaminase domain, an APOBEC3G deaminase domain, an APOBEC3H deaminase domain, or an APOBEC4 deaminase domain. In some embodiments that may be combined with any of the preceding embodiments, the APOBEC family deaminase domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain includes SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain is an AID deaminase domain, and the AID deaminase domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 266, SEQ ID NO: 267, SEQ ID NO: 268, SEQ ID NO: 269, SEQ ID NO: 607, SEQ ID NO: 608, or SEQ ID NO: 1879. In some embodiments that may be combined with any of the preceding embodiments, the AID deaminase domain is SEQ ID NO: 266, SEQ ID NO: 267, SEQ ID NO: 268, SEQ ID NO: 269, SEQ ID NO: 607, SEQ ID NO: 608, or SEQ ID NO: 1879.

In some aspects, the present disclosure relates to a base-editing system including (i) a fusion protein including (a) a Cas12a nickase domain or a dCas12a domain and (b) an APOBEC family deaminase domain; and (ii) a npUGI. In some embodiments that may be combined with any of the preceding embodiments, the APOBEC family deaminase domain is selected from the group of an APOBEC1 deaminase domain, an APOBEC2 deaminase domain, an APOBEC3A deaminase domain, an APOBEC3B deaminase domain, an APOBEC3C deaminase domain, an APOBEC3D deaminase domain, an APOBEC3F deaminase domain, an APOBEC3G deaminase domain, an APOBEC3H deaminase domain, or an APOBEC4 deaminase domain. In some embodiments that may be combined with any of the preceding embodiments, the APOBEC family deaminase domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain includes SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain is an AID deaminase domain, and the AID deaminase domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 266, SEQ ID NO: 267, SEQ ID NO: 268, SEQ ID NO: 269, SEQ ID NO: 607, SEQ ID NO: 608, or SEQ ID NO: 1879. In some embodiments that may be combined with any of the preceding embodiments, the AID deaminase domain is SEQ ID NO: 266, SEQ ID NO: 267, SEQ ID NO: 268, SEQ ID NO: 269, SEQ ID NO: 607, SEQ ID NO: 608, or SEQ ID NO: 1879.

In some aspects, the present disclosure relates to a base-editing system including (i) a fusion protein including (a) a TALE domain and (b) an APOBEC family deaminase domain; and (ii) a npUGI. In some embodiments that may be combined with any of the preceding embodiments, the APOBEC family deaminase domain is selected from the group of an APOBEC1 deaminase domain, an APOBEC2 deaminase domain, an APOBEC3A deaminase domain, an APOBEC3B deaminase domain, an APOBEC3C deaminase domain, an APOBEC3D deaminase domain, an APOBEC3F deaminase domain, an APOBEC3G deaminase domain, an APOBEC3H deaminase domain, or an APOBEC4 deaminase domain. In some embodiments that may be combined with any of the preceding embodiments, the APOBEC family deaminase domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain includes SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain is an AID deaminase domain, and the AID deaminase domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 266, SEQ ID NO: 267, SEQ ID NO: 268, SEQ ID NO: 269, SEQ ID NO: 607, SEQ ID NO: 608, or SEQ ID NO: 1879. In some embodiments that may be combined with any of the preceding embodiments, the AID deaminase domain is SEQ ID NO: 266, SEQ ID NO: 267, SEQ ID NO: 268, SEQ ID NO: 269, SEQ ID NO: 607, SEQ ID NO: 608, or SEQ ID NO: 1879.

In some aspects, the present disclosure relates to a base-editing system including (i) a fusion protein including (a) a ZF domain and (b) an APOBEC family deaminase domain; and (ii) a npUGI. In some embodiments that may be combined with any of the preceding embodiments, the APOBEC family deaminase domain is selected from the group of an APOBEC1 deaminase domain, an APOBEC2 deaminase domain, an APOBEC3A deaminase domain, an APOBEC3B deaminase domain, an APOBEC3C deaminase domain, an APOBEC3D deaminase domain, an APOBEC3F deaminase domain, an APOBEC3G deaminase domain, an APOBEC3H deaminase domain, or an APOBEC4 deaminase domain. In some embodiments that may be combined with any of the preceding embodiments, the APOBEC family deaminase domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain includes SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain is an AID deaminase domain, and the AID deaminase domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 266, SEQ ID NO: 267, SEQ ID NO: 268, SEQ ID NO: 269, SEQ ID NO: 607, SEQ ID NO: 608, or SEQ ID NO: 1879. In some embodiments that may be combined with any of the preceding embodiments, the AID deaminase domain is SEQ ID NO: 266, SEQ ID NO: 267, SEQ ID NO: 268, SEQ ID NO: 269, SEQ ID NO: 607, SEQ ID NO: 608, or SEQ ID NO: 1879.

In some embodiments that may be combined with any of the preceding embodiments, the npUGI is selected from the group of a small molecule inhibitor of uracil-DNA glycosylase (UDG) or a nucleic acid inhibitor of UDG. In some embodiments that may be combined with any of the preceding embodiments, the small molecule inhibitor of UDG is a compound of formula (I):

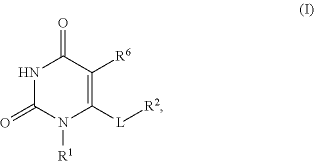

or a pharmaceutically acceptable salt thereof, wherein: R¹ is H, a furanose carbohydrate, a pyranose carbohydrate, a carbohydrate mimetic, C₁₋₁₆alkyl, C₁₋₁₆alkenyl, C₁₋₁₆alkynyl, C₁₋₁₆alkoxy, or C₆₋₂₀aryl, wherein the furanose carbohydrate, or a derivative thereof, pyranose carbohydrate, or a derivative thereof, carbohydrate mimetic, C₁₋₁₆alkyl, C₁₋₁₆alkenyl, C₁₋₁₆alkynyl, C₁₋₁₆alkoxy, or C₆₋₂₀aryl is independently optionally substituted with one or more substituents selected from the group of hydroxyl, halo, cyano, NO₂, N(R⁴)(R⁵), C₁₋₁₆alkoxy, or C₆₋₂₀aryl, wherein the C₆₋₂₀aryl is further optionally substituted with one or more substituents selected from the group of C₁₋₁₆alkyl, C₁₋₁₆alkenyl, C₁₋₁₆alkynyl, C₁₋₁₆alkoxy, hydroxyl, halo, cyano, NO₂, or N(R⁴)(R⁵); L is O, S, or *—N(R³)—**, wherein R³ is H or C₁₋₁₆alkyl, and ** indicates the point of attachment to the R² moiety and * indicates the point of attachment to the remainder of the molecule; R² is H, N(R⁴)(R⁵), or C₆₋₂₀aryl, wherein the C₆₋₂₀aryl is independently substituted with one or more substituents selected from the group of C₁₋₁₆alkyl, C₁₋₁₆alkenyl, C₁₋₁₆alkynyl, or C₁₋₁₆alkoxy; R⁴ and R⁵ are each independently H or C₆₋₂₀aryl; and R⁶ is H or halo. In some embodiments that may be combined with any of the preceding embodiments, the nucleic acid inhibitor of UDG is selected from the group of a double-stranded RNA inhibitor, an antisense oligonucleotide inhibitor, a RNA/DNA hybrid inhibitor, a microRNA inhibitor, or a siRNA inhibitor. In some embodiments that may be combined with any of the preceding embodiments, the nucleic acid inhibitor reversibly inhibits an activity of an UDG. In some embodiments that may be combined with any of the preceding embodiments, the npUGI is a RNA guide of the Cas9 nickase domain, the dCas9 domain, the Cas12a nickase domain, or the dCas12a domain, wherein the RNA guide includes a 5′ end and a 3′ end, and wherein the RNA guide includes one or more deoxyuracil (dU) bases added to the 5′ end or the 3′ end. In some embodiments that may be combined with any of the preceding embodiments, the one or more dU bases are modified to resist glycosidic bond cleavage, have enhanced in vivo stability, or have enhanced ability to bind UDG, as compared to unmodified dU bases. In some embodiments that may be combined with any of the preceding embodiments, the one or more dU bases include a section of double-stranded DNA. In some embodiments that may be combined with any of the preceding embodiments, the section of double-stranded DNA includes one or more U:T pairs, U:G pairs, U:A pairs, U:C pairs, dU:T pairs, dU:G pairs, dU:A pairs, dU:T pairs, or any combination thereof (e.g., any combination of U or dU base mismatch pairs). In some embodiments that may be combined with any of the preceding embodiments, the UDG is an animal UDG. In some embodiments that may be combined with any of the preceding embodiments, the UDG is a plant UDG. In some embodiments that may be combined with any of the preceding embodiments, the plant UDG includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments that may be combined with any of the preceding embodiments, the plant UDG includes SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments that may be combined with any of the preceding embodiments, the fusion protein and the npUGI are chemically linked. In some embodiments that may be combined with any of the preceding embodiments, the fusion protein and the npUGI are not chemically linked.

Some aspects of the disclosure provide a base-editing system including a fusion protein including a DNA binding domain (e.g., a Cas9 domain, a Cas12a domain, a Cas12h domain, a Cas12i domain, a CasX domain, a CasY domain, a C2c1 domain, a C2c2 domain, a C2c3 domain, an Argonaute domain, or a ZF domain) fused to a nucleic acid editing domain, wherein the nucleic acid editing domain is fused to the N-terminus of the DNA binding domain. Some aspects of the disclosure provide a base-editing system including a fusion protein including a DNA binding domain (e.g., a Cas9 domain, a Cas12a domain, a Cas12h domain, a Cas12i domain, a CasX domain, a CasY domain, a C2c1 domain, a C2c2 domain, a C2c3 domain, an Argonaute domain, a TALE domain, or a ZF domain) fused to a nucleic acid editing domain, wherein the nucleic acid editing domain is fused to the C-terminus of the DNA binding domain. Some aspects of the disclosure provide a base-editing system including a fusion protein including a DNA binding domain (e.g., a Cas9 domain, a Cas12a domain, a Cas12h domain, a Cas12i domain, a CasX domain, a CasY domain, a C2c1 domain, a C2c2 domain, a C2c3 domain, an Argonaute domain, a TALE domain, or a ZF domain), wherein the nucleic acid editing domain is insertionally fused to the DNA binding domain. In some embodiments that may be combined with any of the preceding embodiments, the general architecture of exemplary base-editing systems provided herein includes the structure: [NH2]-[nucleic acid editing domain]-[DNA binding domain]-[COOH] or [NH2]-[DNA binding domain]-[nucleic acid editing domain]-[COOH], wherein NH2 is the N-terminus of the base-editing system, and COOH is the C-terminus of the base-editing system. Additional suitable nucleic acid editing domains will be apparent to the skilled artisan based on this disclosure and knowledge in the field.

In some aspects, the base-editing systems provided herein further include (iii) a programmable DNA-binding domain, for example, a ZF domain, a TALE, or a second Cas protein (e.g., a third protein). Without wishing to be bound by any particular theory, fusing a programmable DNA-binding domain (e.g., a Cas9 domain, a Cas12a domain, a Cas12h domain, a Cas12i domain, a CasX domain, a CasY domain, a C2c1 domain, a C2c2 domain, a C2c3 domain, a TALE domain, or a ZF domain) to a base-editing system including a fusion protein including (i) a DNA binding domain (e.g., a first domain); and (ii) a nucleic acid-editing domain (e.g., a second domain) may be useful for improving specificity of the base-editing system to a target nucleic acid sequence. In some embodiments, the third domain is a DNA binding domain (e.g., a Cas9 domain, a Cas12a domain, a Cas12h domain, a Cas12i domain, a CasX domain, a CasY domain, a C2c1 domain, a C2c2 domain, a C2c3 domain, an Argonaute domain, a TALE domain, or a ZF domain). In some embodiments, the third domain is any of the DNA binding domains provided herein. In some embodiments, the third domain is fused to N-terminus of the DNA binding domain or the nucleic acid-editing domain. In some embodiments, the third domain is fused to the C-terminus of the DNA binding domain or the nucleic acid-editing domain. In some embodiments, the third domain is insertionally fused to the of the DNA binding domain or the nucleic acid-editing domain.

In some embodiments, the DNA binding domain (e.g., a Cas9 domain, a Cas12a domain, a Cas12h domain, a Cas12i domain, a CasX domain, a CasY domain, a C2c1 domain, a C2c2 domain, a C2c3 domain, an Argonaute domain, a TALE domain, or a ZF domain) and the nucleic acid editing-editing domain are fused via a linker. In some embodiments, the third domain is fused to the DNA binding domain or the nucleic acid-editing domain via a linker (i.e., a second or third linker). In some embodiments, the linker includes a (GGGS)ₙ(SEQ ID NO: 265), a (GGGGS)ₙ(SEQ ID NO: 5), a (G)ₙ, an (EAAAK) (SEQ ID NO: 6), a (GGS)ₙ, (SGGS)ₙ (SEQ ID NO: 1877), an SGSETPGTSESATPES (SEQ ID NO: 7) motif (see, e.g., Guilinger et al., Nat. Biotechnol (2014) 32(6): 577-82), or an (XP)ₙ motif, or a combination of any of these, wherein n is independently an integer between 1 and 30. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if more than one linker or more than one linker motif is present, any combination thereof. In some embodiments, the linker includes a (GGS)ₙ motif, wherein n is 1, 2, 3, 4, 5, 6, 7, 8, 9, 10, 11, 12, 13, 14 or 15. In some embodiments, the linker includes a (GGS)ₙ motif, wherein n is 1, 3, or 7. In some embodiments, the linker includes the amino acid sequence SGSETPGTSESATPES (SEQ ID NO: 7). Additional suitable linker motifs and linker configurations will be apparent to those of skill in the art. In some embodiments, suitable linker motifs and configurations include those described in Chen et al., Adv Drug Deliv Rev (2013) 65(10): 1357-69. It should be appreciated that any of the proteins provided in any of the general architectures of exemplary base-editing systems may be connected by one or more of the linkers provided herein. In some embodiments, the linkers are the same. In some embodiments, the linkers are different. In some embodiments, one or more of the proteins provided in any of the general architectures of exemplary DNA binding base-editing systems are not fused via a linker.

In some embodiments that may be combined with any of the preceding embodiments, the base-editing system further include a nuclear localization sequence (NLS). In some embodiments, the NLS is located N-terminal of the DNA binding domain or the nucleic acid-editing domain. In some embodiments, the NLS is located C-terminal of the DNA binding domain or the nucleic acid-editing domain. In some embodiments, the NLS is located insertionally to the DNA binding domain or the nucleic acid-editing domain. Nuclear localization sequences are known in the art and would be apparent to the skilled artisan. Exemplary NLS sequences are described in Plank et al., PCT/EP2000/011690. In some embodiments, a NLS includes the amino acid sequence PKKKRKV (SEQ ID NO: 741) or MDSLLMNRRKFLYQFKNVRWAKGRRETYLC (SEQ ID NO: 742). In some embodiments, the NLS is fused to the base-editing system via one or more linkers. In some embodiments, the NLS is fused to the base-editing system without a linker.

Other exemplary features that may be present are localization sequences, such as cytoplasmic localization sequences, export sequences, such as nuclear export sequences, or other localization sequences, as well as sequence tags that are useful for solubilization, purification, or detection of the base-editing systems. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the base-editing system includes one or more His tags. In some embodiments, the localization sequence or tag is fused to the base-editing system via one or more linkers. In some embodiments, the localization sequence or tag is fused to the base-editing system without a linker.

In some embodiments, the base-editing system of the present disclosure further includes a npUGI. In some embodiments, the npUGI is selected from the group including an organochemical inhibitor of uracil glycosylase, a nucleic acid based inhibitor of uracil-DNA glycosylase, or a RNA guide of a dCas nuclease (e.g., dCas9, dCas12a, dCas12h, dCas12i, dCasX, dCasY, dC2c1, dC2c2, or dC2c3) with a poly dU (deoxy-Uracil) extension.

### Methods of Using Base-Editing Systems

In some aspects, the present disclosure relates to methods of editing a target nucleic acid molecule, including contacting a target nucleic acid molecule with the base-editing system of any of the preceding embodiments, wherein the DNA binding domain of the fusion protein is the Cas domain.

In some aspects, the present disclosure relates to methods of editing a target nucleic acid molecule, including contacting a target nucleic acid molecule with the base-editing system of any of the preceding embodiments, wherein the DNA binding domain of the fusion protein is the TALE domain or the ZF domain.

In some embodiments that may be combined with any of the preceding embodiments, the fusion protein and the npUGI of the base-editing system are chemically linked. In some embodiments that may be combined with any of the preceding embodiments, the fusion protein and the npUGI are chemically linked by in vitro complexing or in vivo complexing. In some embodiments that may be combined with any of the preceding embodiments, the fusion protein and the npUGI of the base-editing system are not chemically linked. In some embodiments that may be combined with any of the preceding embodiments, the fusion protein and the npUGI are co-delivered, the fusion protein is delivered before the npUGI, or the npUGI is delivered before the fusion protein. In some embodiments that may be combined with any of the preceding embodiments, the base-editing system provided herein is generated in vitro by complexing. In some embodiments that may be combined with any of the preceding embodiments, the base-editing system provided herein is delivered by PEG-mediated transfection. In some embodiments that may be combined with any of the preceding embodiments, the base-editing system provided herein is generated in vitro by complexing a purified CRISPR-deaminase with the RNA-DNA hybrid guide RNA and is subsequently delivered by PEG-mediated transfection. In some embodiments that may be combined with any of the preceding embodiments, the base-editing system provided herein is vector-encoded. In some embodiments that may be combined with any of the preceding embodiments, the base-editing system provided herein is vector-encoded and co-delivered with exogenous nucleic acid via PEG-mediated transfection. In some embodiments that may be combined with any of the preceding embodiments, the base-editing system provided herein is delivered by PEG-mediated transfection while the small molecule inhibitor is delivered via cell culture media supplementation. In some embodiments that may be combined with any of the preceding embodiments, the target nucleic acid sequence is a double-stranded DNA (dsDNA) sequence, and the target nucleic acid sequence is in the genome of an organism. In some embodiments that may be combined with any of the preceding embodiments, the target nucleic acid sequence is associated with a disease or disorder, and the target nucleic acid sequence includes a point mutation. In some embodiments that may be combined with any of the preceding embodiments, the point mutation is a T to C point mutation, and the T to C point mutation is associated with the disease or disorder. In some embodiments that may be combined with any of the preceding embodiments, the point mutation is a T to C point mutation in a codon of the target nucleic acid sequence, and the point mutation results in an amino acid change in a polypeptide encoded by the target nucleic acid sequence as compared to a wild-type polypeptide encoded by a wild-type nucleic acid sequence without the point mutation. In some embodiments that may be combined with any of the preceding embodiments, contacting the target nucleic acid sequence with the base-editing system produces a nucleic acid sequence that is not associated with the disease or disorder. In some embodiments that may be combined with any of the preceding embodiments, contacting the target nucleic acid sequence with the base-editing system produces a wild-type nucleic acid sequence without the point mutation. In some embodiments that may be combined with any of the preceding embodiments, the disease or disorder is cystic fibrosis, phenylketonuria, epidermolytic hyperkeratosis (EHK), Charcot-Marie-Toot disease type 4J, neuroblastoma (NB), von Willebrand disease (vWD), myotonia congenital, hereditary renal amyloidosis, dilated cardiomyopathy (DCM), hereditary lymphedema, familial Alzheimer's disease, HIV, Prion disease, chronic infantile neurologic cutaneous articular syndrome (CINCA), desmin-related myopathy (DRM), a neoplastic disease associated with a mutant PI3KCA protein, a mutant CTNNBl protein, a mutant HRAS protein, or a mutant p53 protein. In some embodiments that may be combined with any of the preceding embodiments, the contacting is performed in vivo. In some embodiments that may be combined with any of the preceding embodiments, the contacting is performed in vitro. In some embodiments that may be combined with any of the preceding embodiments, the organism is a mammal, and the mammal is selected from the group of mouse, rat, human, pig, cow, chicken, rhesus monkey, or guinea pig. In some embodiments that may be combined with any of the preceding embodiments, the target nucleic acid sequence is selected from the group of a target nucleic acid sequence associated with a disease, a target nucleic acid sequence associated with a disorder, a target nucleic acid sequence associated with metabolic function, a target nucleic acid sequence associated with reproductive function, a target nucleic acid sequence associated with disease resistance function, a target nucleic acid sequence associated with stress tolerance function, a target nucleic acid sequence associated with agronomic function, or a target nucleic acid sequence associated with nutritional function. In some embodiments that may be combined with any of the preceding embodiments, contacting the target nucleic acid sequence with the base-editing system produces a nucleic acid sequence selected from the group of a nucleic acid sequence with a corrected deleterious mutation, a nucleic acid sequence derivative, a modified nucleic acid sequence, a nucleic acid sequence with an inserted sequence, a nucleic acid sequence with improved function, or a nucleic acid sequence with altered function. In some embodiments that may be combined with any of the preceding embodiments, the target nucleic acid sequence includes a point mutation, and contacting the target nucleic acid sequence with the base-editing system produces a wild-type nucleic acid sequence without the point mutation. In some embodiments that may be combined with any of the preceding embodiments, the contacting is performed in vivo. In some embodiments that may be combined with any of the preceding embodiments, the contacting is performed in vitro. In some embodiments that may be combined with any of the preceding embodiments, the organism is a plant, and the plant is selected from the group of sorghum, corn, tomato, rice, soybean, or wheat. In some embodiments that may be combined with any of the preceding embodiments, the organism is a prokaryote.

In some aspects, the present disclosure relates to methods for in vivo editing of a cytosine residue of a target DNA sequence, the method including a) contacting the target DNA sequence with the base-editing system of any one of claims 2-11 and 14-41, wherein the DNA binding domain of the fusion protein is the Cas domain, and a guide nucleic acid; and b) cultivating the cell through a cell division.

In some aspects, the present disclosure relates to methods for editing a nucleobase pair of a double-stranded DNA sequence, the method including: a) contacting a target region of the double-stranded DNA sequence, wherein the target region includes a target nucleobase pair, with the base-editing system of any one of claims 2-11 and 14-41, wherein the DNA binding domain of the fusion protein is the Cas domain, and a guide nucleic acid, wherein the target region includes a target nucleobase pair; b) inducing strand separation of said target region; c) converting a first nucleobase of said target nucleobase pair in a single strand of the target region to a second nucleobase; and d) cutting no more than one strand of said target region; wherein a third nucleobase complementary to the first nucleobase base is replaced by a fourth nucleobase complementary to the second nucleobase and the method causes less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.5%, less than 0.2%, or less than 0.1% indel formation in the double-stranded DNA sequence. In some embodiments that may be combined with any of the preceding embodiments, the cut single strand is hybridized to the guide nucleic acid. In some embodiments that may be combined with any of the preceding embodiments, the cut single strand is opposite to the strand including the first nucleobase. In some embodiments that may be combined with any of the preceding embodiments, the first base is a cytosine. In some embodiments that may be combined with any of the preceding embodiments, the second base is a uracil. In some embodiments that may be combined with any of the preceding embodiments, the Cas domain is selected from the group of a Cas nickase domain or a deactivated Cas domain.

In some aspects, the present disclosure relates to methods for editing a nucleobase pair of a double-stranded DNA sequence, the method including: a) contacting a target region of the double-stranded DNA sequence, wherein the target region includes a target nucleobase pair, with the base-editing system of any of claims 2-11 and 14-41, wherein the DNA binding domain of the fusion protein is the Cas domain, and a guide nucleic acid; b) inducing strand separation of said target region; c) converting a first nucleobase of said target nucleobase pair in a single strand of the target region to a second nucleobase; d) cutting no more than one strand of said target region; wherein a third nucleobase complementary to the first nucleobase base is replaced by a fourth nucleobase complementary to the second nucleobase; and e) replacing the second nucleobase with a fifth nucleobase that is complementary to the fourth nucleobase, thereby generating an intended edited basepair, wherein the efficiency of generating the intended edited basepair is at least 5%, at least 10%, at least 15%, at least 20%, at least 25%, at least 30%, at least 35%, at least 40%, at least 45%, at least 50%, at least 55%, at least 60%, at least 65%, or at least 70%. In some embodiments that may be combined with any of the preceding embodiments, the cut single strand is hybridized to the guide nucleic acid. In some embodiments that may be combined with any of the preceding embodiments, the cut single strand is opposite to the strand including the first nucleobase. In some embodiments that may be combined with any of the preceding embodiments, the first base is a cytosine. In some embodiments that may be combined with any of the preceding embodiments, the second base is an uracil. In some embodiments that may be combined with any of the preceding embodiments, the Cas domain is selected from the group of a Cas nickase domain or a deactivated Cas domain.

In some aspects, the present disclosure relates to methods for editing a nucleobase pair of a double-stranded DNA sequence, the method including: a) contacting a target region of the double-stranded DNA sequence, wherein the target region includes a target nucleobase pair, with the base-editing system of any of claims 2-11 and 14-41, wherein the DNA binding domain of the fusion protein is the Cas domain, and a guide nucleic acid; b) converting a first nucleobase of said target nucleobase pair of the target region to a second nucleobase; and c) inhibiting base excision repair of the second nucleobase.

In some aspects, the present disclosure relates to methods for editing a nucleobase pair of a double-stranded DNA sequence, the method including: a) contacting a target region of the double-stranded DNA sequence, wherein the target region includes a target nucleobase pair, with the base-editing system of any of claims 2-11 and 14-41, wherein the DNA binding domain of the fusion protein is the Cas domain, and a guide nucleic acid; b) converting a first nucleobase of said target nucleobase pair in the target region to a second nucleobase; and c) initiating mismatch repair to convert the nucleobase complementary to the first nucleobase on the non-edited strand to a nucleobase complementary to the second nucleobase.

In some embodiments that may be combined with any of the preceding embodiments, the Cas domain is selected from the group of a Cas 9 domain, a Cas12a domain, a Cas12h domain, a Cas12i domain, a CasX domain, a CasY domain, a C2c1 domain, a C2c2 domain, or a C2c3 domain. In some embodiments that may be combined with any of the preceding embodiments, the Cas domain is the Cas12a domain, and the Cas12a domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413. In some embodiments that may be combined with any of the preceding embodiments, the Cas12a domain is SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413. In some embodiments that may be combined with any of the preceding embodiments, the Cas domain is the Cas9 domain, and the Cas9 domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 325, SEQ ID NO: 674, SEQ ID NO: 1869, SEQ ID NO: 1870, SEQ ID NO: 1871, SEQ ID NO: 1872, SEQ ID NO: 1873, SEQ ID NO: 1874, SEQ ID NO: 1875, or SEQ ID NO: 1876. In some embodiments that may be combined with any of the preceding embodiments, the Cas9 domain is SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 325, SEQ ID NO: 674, SEQ ID NO: 1869, SEQ ID NO: 1870, SEQ ID NO: 1871, SEQ ID NO: 1872, SEQ ID NO: 1873, SEQ ID NO: 1874, SEQ ID NO: 1875, or SEQ ID NO: 1876. In some embodiments that may be combined with any of the preceding embodiments, the Cas9 domain is SEQ ID NO: 674.

In some aspects, the present disclosure relates to methods for in vivo editing of a cytosine residue of a target DNA sequence, the method including a) contacting the target DNA sequence with the base-editing system of any one of claims 12-41, wherein the DNA binding domain of the fusion protein is the TALE domain or the ZF domain; and b) cultivating the cell through a cell division.

In some embodiments that may be combined with any of the preceding embodiments, the cytidine deaminase domain is an APOBEC family deaminase domain. In some embodiments that may be combined with any of the preceding embodiments, the APOBEC family deaminase domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain is SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments that may be combined with any of the preceding embodiments, the APOBEC family deaminase domain is an activation-induced deaminase (AID) domain. In some embodiments that may be combined with any of the preceding embodiments, the AID deaminase domain includes an amino acid sequence with at least 80% sequence identity, at least 82.5% sequence identity, at least 85% sequence identity, at least 87.5% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.5% sequence identity, or at least 99.9% sequence identity to SEQ ID NO: 266, SEQ ID NO: 267, SEQ ID NO: 268, SEQ ID NO: 269, SEQ ID NO: 607, SEQ ID NO: 608, or SEQ ID NO: 1879. In some embodiments that may be combined with any of the preceding embodiments, the AID deaminase domain is SEQ ID NO: 266, SEQ ID NO: 267, SEQ ID NO: 268, SEQ ID NO: 269, SEQ ID NO: 607, SEQ ID NO: 608, or SEQ ID NO: 1879. In some embodiments that may be combined with any of the preceding embodiments, the npUGI of the base-editing system is selected from the group of a small molecule inhibitor of uracil-DNA glycosylase (UDG) or a nucleic acid inhibitor of UDG. In some embodiments that may be combined with any of the preceding embodiments, the Cas domain is the Cas nickase domain or the deactivated Cas domain, the npUGI of the base-editing system is a RNA guide of the Cas nickase domain or the deactivated Cas domain, the RNA guide has a 5′ end and a 3′ end, and the RNA guide has one or more deoxyuracil (dU) bases added to the 5′ end or the 3′ end. In some embodiments that may be combined with any of the preceding embodiments, the fusion protein and the npUGI are chemically linked. In some embodiments that may be combined with any of the preceding embodiments, the fusion protein and the npUGI are chemically linked by in vitro complexing or in vivo complexing. In some embodiments that may be combined with any of the preceding embodiments, the fusion protein and the npUGI are not chemically linked. In some embodiments that may be combined with any of the preceding embodiments, the fusion protein and the npUGI are co-delivered, the fusion protein is delivered before the npUGI, or the npUGI is delivered before the fusion protein. In some embodiments that may be combined with any of the preceding embodiments, the base-editing system provided herein is generated in vitro by complexing. In some embodiments that may be combined with any of the preceding embodiments, the base-editing system provided herein is delivered by PEG-mediated transfection. In some embodiments that may be combined with any of the preceding embodiments, the base-editing system provided herein is generated in vitro by complexing a purified CRISPR-deaminase with the RNA-DNA hybrid guide RNA and is subsequently delivered by PEG-mediated transfection. In some embodiments that may be combined with any of the preceding embodiments, the base-editing system provided herein is vector-encoded. In some embodiments that may be combined with any of the preceding embodiments, the base-editing system provided herein is vector-encoded and co-delivered with exogenous nucleic acid via PEG-mediated transfection. In some embodiments that may be combined with any of the preceding embodiments, the base-editing system provided herein is delivered by PEG-mediated transfection while the small molecule inhibitor is delivered via cell culture media supplementation. In some embodiments that may be combined with any of the preceding embodiments, the DNA sequence is in the genome of an organism. In some embodiments that may be combined with any of the preceding embodiments, the organism is a mammal selected from the group of mouse, rat, human, pig, cow, chicken, rhesus monkey, or guinea pig. In some embodiments that may be combined with any of the preceding embodiments, the organism is a plant selected from the group of sorghum, corn, tomato, rice, soybean, or wheat.

### DNA Binding Domains

In some aspects, the present disclosure relates to DNA binding domains or DNA sequence targeting components. In some embodiments, the DNA binding domain or DNA sequence-targeting component is a Cas DNA endonuclease (e.g., a Cas domain), a transcription activator-like effector (TALE) (e.g., a TALE domain), a Zinc Finger (ZF) (e.g., a ZF domain), a meganuclease (e.g., a meganuclease domain), or an Argonaute (e.g., an Argonaute domain). In some embodiments, the Cas domain is a Cas9 domain, a nuclease inactive Cas9 (dCas9) domain, a Cas9 nickase domain, a Cas12a domain, a nuclease inactive Cas12a (dCas12a) domain, a Cas12a nickase domain, a Cas12h domain, a Cas12i domain, a CasX domain, a CasY domain, a C2c1 domain, a C2c2 domain, or a C2c3 domain.

In some embodiments, the DNA binding domain of any of the base-editing systems provided herein is a single effector of a microbial CRISPR-Cas system. Single effectors of microbial CRISPR-Cas systems include, without limitation, Cas9, Cas12a, Cas12h, Cas12i, CasX, CasY, C2c1, C2c2, and C2c3. Typically, microbial CRISPR-Cas systems are divided into Class 1 and Class 2 systems. Class 1 systems have multisubunit effector complexes, while Class 2 systems have a single protein effector. Cas9 and Cas12a are Class 2 effectors.

The term “RNA-programmable nuclease,” and “RNA-guided nuclease” are used interchangeably herein and refer to a nuclease that forms a complex with (e.g., binds or associates with) one or more RNA that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species include two domains: (1) a domain that shares homology to a target nucleic acid and directs binding of a Cas complex (e.g., a Cas9 complex, Cas12a complex, a Cas12h complex, a Cas12i complex, a CasX complex, a CasY complex, a C2c1 complex, a C2c2 complex, or a C2c3 complex) to the target; and (2) a domain that binds a Cas protein (e.g., a Cas9 protein, a Cas12a protein, a Cas12h protein, a Cas12i protein, a CasX protein, a CasY protein, a C2c1 protein, a C2c2 protein, or a C2c3 protein). In some embodiments, domain (2) corresponds to a sequence known as a tracrRNA, and includes a stem-loop structure. For example, in some embodiments, domain (2) is identical or homologous to a tracrRNA as provided in Jinek et al., Science (2012) 337:816-821. Other examples of gRNAs (e.g., those including domain 2) can be found in U.S. Provisional Patent Application Ser. No. 61/874,682, filed Sep. 6, 2013; and U.S. Provisional Patent Application Ser. No. 61/874,746, filed Sep. 6, 2013. In some embodiments, a gRNA includes two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA includes a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from S. pyogenes (see, e.g., Ferretti et al., PNAS (2001) 98:4658-4663; Deltcheva et al., Nature (2011) 471:602-607; and Jinek et al., Science (2012) 337:816-821). In some embodiments, the RNA-programmable nuclease is the (CRISPR-associated system) Cas12a endonuclease, for example Cas12a from Lachnospiraceae bacterium. Cas domain containing base-editing systems can include one or more RNA sequences, or DNA sequences encoding the RNA sequences. The RNA sequences may be fused at least temporarily to the guide RNA (crRNA), or be expressed separately therefrom. Examples of such sequences can include pre-crRNAs, tracR sequences or putative tracR sequences, direct repeat RNAs of corresponding CRISPR arrays, RNA sequences mediating tethering, RNAi or siRNA sequences mediating silencing of one or more endogenous genes, as well as RNA-processing regions like tRNAs or ribozymes.

Because RNA-programmable nucleases (e.g., Cas9, Cas12a, Cas12h, Cas12i, CasX, CasY, C2c1, C2c2, or C2c3) use RNA:DNA hybridization to target DNA cleavage sites, these proteins are able to be targeted, in principle, to any sequence specified by the guide RNA. Methods of using RNA-programmable nucleases, such as Cas (e.g., Cas9, Cas12a, Cas12h, Cas12i, CasX, CasY, C2c1, C2c2, or C2c3), for site-specific cleavage (e.g., to modify a genome) are known in the art (see e.g., Cong et al., Science 339 (2013), 819-823; Mali et al., Science (2013) 339, 823-826; Hwang, et al., Nat Biotechnol (2013) 31, 227-229; Jinek et al., eLife (2013) 2, e00471; Dicarlo et al., Nucleic Acids Res (2013); Jiang et al., Nat Biotechnol (2013) 31, 233-239).

Any of the names of the Cas domains may be modified by the initials of the organism from which it has been derived. For example, Streptococcus pyogenes Cas9 may be referred to as SpCas9, Lachnospiraceae bacterium Cas12a may be referred to as LbCas12a, and Acidaminococcus sp. Cas12a may be referred to as AsCas12a.

### Cas9 Domains

The clustered regularly interspaced short palindromic repeat (CRISPR) system is a recently discovered prokaryotic adaptive immune system (Jansen et al., Mol Microbiol (2002) 43(6): 1565-75. PMID: 11952905) that has been modified to enable robust and general genome engineering in a variety of organisms and cell lines (Mali et al., Nat Methods (2013) 10(10):957-63. PMID: 24076990). CRISPR-Cas (CRISPR associated) systems are protein-RNA complexes that use an RNA molecule (sgRNA) as a guide to localize the complex to a target DNA sequence via base-pairing (Jore et al., Nat Struct Mol Biol (2011) 18(5):529-36. PMID: 21460843). In the natural systems, a Cas protein then acts as an endonuclease to cleave the targeted DNA sequence (Horvath and Barrangou, Science (2010) 327(5962): 167-70. PMID: 20056882). The target DNA sequence must be both complementary to the sgRNA, and also contain a “protospacer-adjacent motif (PAM) at the 3′-end of the complementary region in order for the system to function (Wiedenheft et al., Nature (2012) 482(7385):331-8. PMID: 22337052).

Among the known Cas proteins, S. pyogenes Cas9 has been mostly widely used as a tool for genome engineering (Gasiunas et al., Trends Microbiol (2013) 21(1.1):562-7. PMID: 24095303). Point mutations can be introduced into a Cas protein to abolish nuclease activity, resulting in a dead Cas (dCas) that still retains its ability to bind DNA in a sgRNA-programmed manner (Qi et al., Cell (2013) 152(5): 1173-83. PMID: 23452860). In principle, when fused to another protein or domain, a dCas can target that protein to virtually any DNA sequence simply by co-expression with an appropriate sgRNA (Mali et al, Nat Methods (2013) 10(10):957-63. PMID: 24076990; Qi et al., Cell (2013) 152(5): 1173-83. PMID: 23452860; Perez-Pinera et al., Nat Methods (2013) 10(10):973-6. PMID: 23892895; Mali et al., Nat Biotechnol (2013) 31(9):833-8. PMID: 23907171; Gilbert et al., Cell (2013) 154(2):442-51. PMID: 23849981; Larson et al., Nat Protoc (2013) 8(11):2180-96. PMID: 24136345; Mali et al., Science (2013) 339(6121):823-6. PMID: 23287722).

In some embodiments, the present disclosure relates to Cas9 domains. The Cas9 domain may be a nuclease active Cas9 domain, a nuclease inactive Cas9 domain (dCas9), or a Cas9 nickase. The term “Cas9” or “Cas9 nuclease” refers to an RNA-guided nuclease including a Cas9 protein, or a portion thereof (e.g., a protein including an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease (see, e.g., Charpentier and Doudna, Nature (2013) 495, (7439):50-1. PMID: 23467164). CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species (see, e.g., Jinek et al, Science (2012) 337:816-821). Cas9 recognizes a short motif in the CRISPR repeat sequences (the PAM or protospacer adjacent motif) to help distinguish self versus non-self Cas9 nuclease sequences and structures are well known to those of skill in the art (see, e.g., Ferretti et al, PNAS (2001) 98:4658-4663; Deltcheva et al., Nature (2011) 471:602-607; and Jinek et al., Science (2012) 337:816-821). Cas9 orthologs have been described in various species, including, but not limited to, Streptococcus pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski et al., RNA Biology (2013) 10:5, 726-737. In some embodiments, a Cas9 nuclease has an inactive (e.g., an inactivated) DNA cleavage domain, that is, the Cas9 is a nickase.

A nuclease-inactivated Cas9 domain, or deactivated Cas9 domain, may interchangeably be referred to as a “dCas9” domain (for nuclease-“dead” Cas9). Methods for generating a Cas9 protein (or a portion thereof) having an inactive DNA cleavage domain are known (See, e.g., Jinek et al., Science (2012) 337:816-821; Qi et al., Cell (2013) 28; 152(5): 1173-83). For example, the DNA cleavage domain of Cas9 is known to include two subdomains, the HNH nuclease subdomain and the RuvCl subdomain. The HNH subdomain cleaves the strand complementary to the gRNA, whereas the RuvCl subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science (2012) 337:816-821; Qi et al, Cell (2013) 28; 152(5): 1173-83).

In some embodiments, the Cas9 domain is a nuclease active domain. For example, the Cas9 domain may be a Cas9 domain that cuts both strands of a duplexed nucleic acid (e.g., both strands of a duplexed DNA molecule). In some embodiments, the Cas9 domain includes an amino acid sequence that is at least 60% identical, at least 65% identical, at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to any one of the amino acid sequences set forth in SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, or SEQ ID NO: 263. In some embodiments, the Cas9 domain includes any one of the amino acid sequences as set forth in SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, or SEQ ID NO: 263. In some embodiments, the Cas9 domain includes an amino acid sequence that has 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more mutations compared to any one of the amino acid sequences set forth in SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, or SEQ ID NO: 263. In some embodiments, the Cas9 domain includes an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth in SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, or SEQ ID NO: 263.

In some embodiments, the Cas9 domain is a nuclease inactive Cas9 domain (dCas9). For example, the dCas9 domain may bind to a duplexed nucleic acid molecule (e.g., via a gRNA molecule) without cleaving either strand of the duplexed nucleic acid molecule. In some embodiments, dCas9 corresponds to, in part or in whole, a Cas9 amino acid sequence having one or more mutations that inactivate the Cas9 nuclease activity. For example, in some embodiments, a dCas9 domain includes a D10A and/or a H840A mutation (SEQ ID NO: 9). In some embodiments, the nuclease inactive dCas9 domain includes a DIOX mutation and a H840X mutation of the amino acid sequence set forth in SEQ ID NO: 10, or a corresponding mutation in any of the Cas9 sequences provided herein, wherein X is any amino acid change. In some embodiments, the nuclease inactive dCas9 domain includes a D10A mutation and a H840A mutation of the amino acid sequence set forth in SEQ ID NO: 10, or a corresponding mutation in any of the Cas9 sequences provided herein. As one example, a nuclease inactive Cas9 domain includes the amino acid sequence set forth in SEQ ID NO: 263 (Cloning vector pPlatTET-gRNA2, Accession No. BAV54124; see, e.g., Qi et al., Cell (2013) 152(5): 1173-83).

In other embodiments, dCas9 variants having mutations other than D10A and H840A are provided, which, e.g., result in nuclease inactivated Cas9 (dCas9). Such mutations, by way of example, include other amino acid substitutions at D10 and H820, or other substitutions within the nuclease domains of Cas9 (e.g., substitutions in the UNH nuclease subdomain and/or the RuvCl subdomain). In some embodiments, variants or homologues of dCas9 (e.g., variants of SEQ ID NO: 10) are provided which are at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to SEQ ID NO: 10. In some embodiments, variants of dCas9 (e.g., variants of SEQ ID NO: 10) are provided having amino acid sequences which are shorter or longer than SEQ ID NO: 10 by about 5 or more, by about or more, by about 15 or more, by about 20 or more, by about 25 or more, by about 30 or more, by about 40 or more, by about 50 or more, by about 75 or more, or by about 100 or more amino acids.

Additional suitable dCas9 domains will be apparent to those of skill in the art based on this disclosure and knowledge in the field, and are within the scope of this disclosure. Such additional exemplary suitable dCas9 domains include, but are not limited to, D10A/H840A, D10A/D839A/H840A, and D10A/D839A/H840A/N863A mutant domains (See, e.g., Prashant et al., Nat Biotechnol (2013) 31(9): 833-838). In some embodiments, the dCas9 domain includes an amino acid sequence that is at least 60% identical, at least 65% identical, at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to any one of the dCas9 domains provided herein.

In some embodiments, the Cas9 domain is a Cas9 nickase. The Cas9 nickase may be a Cas9 protein that is capable of cleaving only one strand of a duplexed nucleic acid molecule (e.g., a duplexed DNA molecule). In some embodiments, the Cas9 nickase cleaves the target strand of a duplexed nucleic acid molecule, meaning that the Cas9 nickase cleaves the strand that is base paired to (complementary to) a gRNA (e.g., an sgRNA) that is bound to the Cas9. In some embodiments, a Cas9 nickase includes a D10A mutation and has a histidine at position 840 of SEQ ID NO: 10, or a mutation in or a corresponding mutation in any of the Cas9 sequences provided herein. Without wishing to be bound by theory, the presence of the catalytic residue H840 restores the activity of the Cas9 to cleave the non-edited (e.g., non-deaminated) strand containing a G opposite the targeted C. Restoration of H840 (e.g., from A840) does not result in the cleavage of the target strand containing the C. For example, a Cas9 nickase may include the amino acid sequence as set forth in SEQ ID NO: 674. In some embodiments, a Cas9 nickase has an active UNH nuclease domain and is able to cleave the non-targeted strand of DNA, i.e., the strand bound by the gRNA. In further embodiments, a Cas9 nickase has an inactive RuvC nuclease domain and is not able to cleave the targeted strand of the DNA, i.e., the strand where base editing is desired. In some embodiments the Cas9 nickase cleaves the non-target, non-base-edited strand of a duplexed nucleic acid molecule, meaning that the Cas9 nickase cleaves the strand that is not base paired to a gRNA (e.g., an sgRNA) that is bound to the Cas9. In some embodiments, a Cas9 nickase includes an H840A mutation and has an aspartic acid residue at position 10 of SEQ ID NO: 10, or a corresponding mutation in any of the Cas9 sequences provided herein. In some embodiments the Cas9 nickase includes an amino acid sequence that is at least 60% identical, at least 65% identical, at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to any one of the Cas9 nickases provided herein. Additional suitable Cas9 nickases will be apparent to those of skill in the art based on this disclosure and knowledge in the field, and are within the scope of this disclosure. Cas9 nickases of the present disclosure are able to generate a single-strand DNA break (nick) at a specific location based on the gRNA-defined target sequence, leading to repair of the non-edited strand, ultimately resulting in a G to A change on the non-edited strand. Briefly, the base-editing system of the present disclosure allows the C of a C-G basepair to be deaminated to a U by a deaminase, e.g., an APOBEC deaminase. Nicking the non-edited strand, having the G, facilitates removal of the G via mismatch repair mechanisms. The npUGI of the base-editing system inhibits UDG, which prevents removal of the U.

In some embodiments, base-editing systems as provided herein include the full-length amino acid sequence of a Cas9 protein, e.g., one of the Cas9 sequences provided herein. In other embodiments, base-editing systems as provided herein do not include a full-length Cas9 sequence, but only a portion thereof. For example, in some embodiments, a base-editing system provided herein includes a portion of a Cas9 protein, wherein the portion binds crRNA and tracrRNA or sgRNA, but does not include a functional nuclease domain, e.g., the portion of the Cas9 protein includes only a truncated version of a nuclease domain or no nuclease domain at all. Exemplary amino acid sequences of suitable Cas9 domains and portions thereof are provided herein, and additional suitable sequences of Cas9 domains and portions thereof will be apparent to those of skill in the art.

In some embodiments, proteins including portions of Cas9 are provided. For example, in some embodiments, a protein includes one of two Cas9 domains: (1) the gRNA binding domain of Cas9; or (2) the DNA cleavage domain of Cas9. In some embodiments, proteins including Cas9 or portions thereof are referred to as “Cas9 variants.” A Cas9 variant shares homology to Cas9, or a portion thereof. For example, a Cas9 variant is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to wild type Cas9. In some embodiments, the Cas9 variant may have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more amino acid changes compared to wild type Cas9. In some embodiments, the Cas9 variant includes a portion of Cas9 (e.g., a gRNA binding domain or a DNA-cleavage domain), such that the portion of the Cas9 is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the corresponding fragment of wild type Cas9. In some embodiments, the portion of Cas9 is at least 50%, at least 55%, at least 60%, at least 65%, at least 70%, at least 72.5%, at least 75%, at least 77.5%, at least 80%, at least 82.5%, at least 85%, at least 87.5%, at least 90%, at least 91%, at least 92%, at least 93%, at least 94%, at least 95%, at least 96%, at least 97%, at least 98%, at least 99%, at least 99.5%, or at least 99.9% of the amino acid length of a corresponding wild type Cas9.

In some embodiments, the Cas9 portion is at least 100 amino acids in length. In some embodiments, the Cas9 portion is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 amino acids in length.

In some embodiments, wild type Cas9 corresponds to Cas9 from Streptococcus pyogenes (NCBI Reference Sequence: NC_017053. 1, SEQ ID NO: 1 (nucleotide); SEQ ID NO:2 (amino acid)). In some embodiments, wild type Cas9 corresponds to SEQ ID NO:3 (nucleotide) and/or SEQ ID NO: 4 (amino acid). In some embodiments, wild type Cas9 corresponds to Cas9 from S. pyogenes (NCBI Reference Sequence: NC_002737.2, SEQ ID NO: 8 (nucleotide); and Uniprot Reference Sequence: Q99ZW2, SEQ ID NO: 10 (amino acid).

In some embodiments, wild type Cas9 refers to Cas9 from: Corynebacterium ulcerans (NCBI Refs: NC_015683.1, NC_017317.1); Corynebacterium diphtheria (NCBI Refs: NC_016782. 1, NC_016786. 1); Spiroplasma syrphidicola (NCBI Ref: NC_021284. 1); Prevotella intermedia (NCBI Ref: NC 017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832. 1), Listeria innocua (NCBI Ref: NP_472073.1), Campylobacter jejuni (NCBI Ref: YP 002344900. 1) or Neisseria meningitidis (NCBI Ref: YP 002342 100.1).

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base-editing system provided herein may need to be placed at a precise location, for example where a target base is placed within a 4 base region (e.g., a “deamination window”), which is approximately 15 bases upstream of the PAM (see, e.g., Komor et al., Nature (2016) 533, 420-424). Accordingly, in some embodiments, any of the base-editing systems provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver et al., Nature (2015) 523, 481-485; and Kleinstiver et al., Nat Biotechnol (2015) 33, 1293-1298.

In some embodiments, the Cas9 domain is a Cas9 domain from S. aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 includes the amino acid sequence SEQ ID NO: 1869. In some embodiments, the SaCas9 includes a N579X mutation of SEQ ID NO: 1869, or a corresponding mutation in any of the Cas9 sequences provided herein, wherein X is any amino acid except for N. In some embodiments, the SaCas9 includes a N579A mutation of SEQ ID NO: 1869, or a corresponding mutation in any of the Cas9 sequences provided herein. In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT PAM sequence. In some embodiments, the SaCas9 domain includes one or more of a E781X, a N967X, and a R1014X mutation of SEQ ID NO: 1869, or a corresponding mutation in any of the Cas9 sequences provided herein, wherein X is any amino acid. In some embodiments, the SaCas9 domain includes one or more of a E781K, a N967K, and a R1014H mutation of SEQ ID NO: 1869, or one or more corresponding mutation in any of the Cas9 sequences provided herein. In some embodiments, the SaCas9 domain includes a E781K, a N967K, or a R1014H mutation of SEQ ID NO: 1869, or corresponding mutations in any of the Cas9 sequences provided herein.

In some embodiments, the Cas9 domain of any of the base-editing systems provided herein includes an amino acid sequence that is at least 60% identical, at least 65% identical, at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to any one of SEQ ID NO: 1869, SEQ ID NO: 1870, or SEQ ID NO: 1871. In some embodiments, the Cas9 domain of any of the base-editing systems provided herein includes the amino acid sequence of any one of SEQ ID NO: 1869, SEQ ID NO: 1870, or SEQ ID NO: 1871. In some embodiments, the Cas9 domain of any of the base-editing systems provided herein includes the amino acid sequence of any one of SEQ ID NO: 1869, SEQ ID NO: 1870, or SEQ ID NO: 1871. Residue N579 of SEQ ID NO: 1869 may be mutated (e.g., to a A579) to yield a SaCas9 nickase. Residue A579 of SEQ ID NO: 1870 can be mutated from N579 to yield a SaCas9 nickase. Residue A579 of SEQ ID NO: 1871(SaKKH Cas9) may be mutated from N579) to yield a SaCas9 nickase. Residues K781, K967, and H1014 of SEQ ID NO: 1871 can be mutated from E781, N967, and R1014 to yield a SaKKH Cas9.

In some embodiments, the Cas9 domain is a Cas9 domain from S. pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 includes the amino acid sequence SEQ ID NO: 1872. In some embodiments, the SpCas9 includes a D9X mutation of SEQ ID NO: 1872, or a corresponding mutation in any of Cas9 sequences provided herein, wherein X is any amino acid except for D. In some embodiments, the SpCas9 includes a D9A mutation of SEQ ID NO: 1872, or a corresponding mutation in any of the Cas9 sequences provided herein. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having a NGG, a NGA, or a NGCG PAM sequence. In some embodiments, the SpCas9 domain includes one or more of a D1134X, a R1334X, and a T1336X mutation of SEQ ID NO: 1872, or a corresponding mutation in any of the Cas9 sequences provided herein, wherein X is any amino acid. In some embodiments, the SpCas9 domain includes one or more of a D1134E, R1334Q, and T1336R mutation of SEQ ID NO: 1872, or a corresponding mutation in any of the Cas9 sequences provided herein. In some embodiments, the SpCas9 domain includes a D1134E, a R1334Q, and a T1336R mutation of SEQ ID NO: 1872, or corresponding mutations in any of the Cas9 sequences provided herein. In some embodiments, the SpCas9 domain includes one or more of a D1134X, a R1334X, and a T1336X mutation of SEQ ID NO: 1872, or a corresponding mutation in any of the Cas9 sequences provided herein, wherein X is any amino acid. In some embodiments, the SpCas9 domain includes one or more of a D1134V, a R1334Q, and a T1336R mutation of SEQ ID NO: 1872, or a corresponding mutation in any of Cas9 sequences provided herein. In some embodiments, the SpCas9 domain includes a D1134V, a R1334Q, and a T1336R mutation of SEQ ID NO: 1872, or corresponding mutations in any of the Cas9 sequences provided herein. In some embodiments, the SpCas9 domain includes one or more of a D1134X, a G1217X, a R1334X, and a T1336X mutation of SEQ ID NO: 1872, or a corresponding mutation in any of the Cas9 sequences provided herein, wherein X is any amino acid. In some embodiments, the SpCas9 domain includes one or more of a D1134V, a G1217R, a R1334Q, and a T1336R mutation of SEQ ID NO: 1872, or a corresponding mutation in any of the Cas9 sequences provided herein. In some embodiments, the SpCas9 domain includes a D1134V, a G1217R, a R1334Q, and a T1336R mutation of SEQ ID NO: 1872, or corresponding mutations in any of the Cas9 sequences provided herein.

In some embodiments, the Cas9 domain of any of the base-editing systems provided herein includes an amino acid sequence that is at least 60% identical, at least 65% identical, at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to any one of SEQ ID NO: 1872, SEQ ID NO: 1873, SEQ ID NO: 1874, SEQ ID NO: 1875, or SEQ ID NO: 1876. In some embodiments, the Cas9 domain of any of the base-editing systems provided herein includes the amino acid sequence of any one of SEQ ID NO: 1872, SEQ ID NO: 1873, SEQ ID NO: 1874, SEQ ID NO: 1875, or SEQ ID NO: 1876. In some embodiments, the Cas9 domain of any of the base-editing systems provided herein includes the amino acid sequence of any one of SEQ ID NO: 1872, SEQ ID NO: 1873, SEQ ID NO: 1874, SEQ ID NO: 1875, or SEQ ID NO: 1876. Residues E1134, Q1334, and R1336 of SEQ ID NO: 1874 can be mutated from D1134, R1334, and T1336 to yield a SpEQR Cas9. Residues V1134, Q1334, and R1336 of SEQ ID NO: 1875 can be mutated from D1134, R1334, and T1336 to yield a SpVQR Cas9. Residues V1134, R1217, Q1334, and R1336 of SEQ ID NO: 1876 can be mutated from D1134, G1217, R1334, and T1336 to yield a SpVRER Cas9.

Some aspects of the disclosure provide base-editing systems (e.g., any of the base-editing systems provided herein) including a Cas9 domain that has high fidelity. Additional aspects of the disclosure provide base-editing systems (e.g., any of the base-editing systems provided herein) including a Cas9 domain with decreased electrostatic interactions between the Cas9 domain and a sugar-phosphate backbone of a DNA, as compared to a wild-type Cas9 domain. In some embodiments, a Cas9 domain (e.g., a wild type Cas9 domain) includes one or more mutations that decreases the association between the Cas9 domain and a sugar-phosphate backbone of a DNA. In some embodiments, any of the base-editing systems provided herein include one or more of a N497X, a R661X, a Q695X, and/or a Q926X mutation of the amino acid sequence provided in SEQ ID NO: 10, or a corresponding mutation in any of the Cas9 sequences provided herein, wherein X is any amino acid. In some embodiments, any of the base-editing systems provided herein include one or more of a N497A, a R661A, a Q695A, and/or a Q926A mutation of the amino acid sequence provided in SEQ ID NO: 10, or a corresponding mutation in any of the Cas9 sequences provided herein. In some embodiments, the Cas9 domain includes a D10A mutation of the amino acid sequence provided in SEQ ID NO: 10, or a corresponding mutation in any of the Cas9 sequences provided herein. In some embodiments, the Cas9 domain (e.g., of any of the base-editing systems provided herein) includes the amino acid sequence as set forth in SEQ ID NO: 325. Cas9 domains with high fidelity are known in the art and would be apparent to the skilled artisan. For example, Cas9 domains with high fidelity have been described in Kleinstiver et al., Nature (2016) 529, 490-495; and Slaymaker et al., Science (2015) 351, 84-88.

### Cas12a Domains

Cas12a is an RNA-guided endonuclease of a class II CRISPR/Cas system. The Cas12a locus contains a mixed alpha/beta domain, a RuvC-I followed by a helical region, a RuvC-II and a zinc finger-like domain. The Cas12a protein has a RuvC-like endonuclease domain that is similar to the RuvC domain of Cas9. In contrast to Cas9, Cas12a does not have a HNH endonuclease domain, and the N-terminal of Cas12a does not have an alpha-helical recognition lobe (see, e.g., Kleinstiver et al., Nat Biotechnol. (2016) 34(8):869-74. PMID: 27347757; and Kim et al., Nat Biotechnol. (2016) 34(8):863-8. PMID: 27272384; Zetsche et al., Cell (2015) 163, 759-771. PMID: 26422227; and Shmakov et al., Mol Cell. (2015) 60(3): 385-97. PMID: 26593719).

Cas12a is an endonuclease of the new CRISPR system that is distinct from Cas9. Cas12a is relatively small in size and can act by a single 42-nt crRNA, which has 23 nt at its 3′ end that are complementary to the protospacer of the target DNA sequence. Further, Cas12a recognizes TTTN PAMs that are positioned 5′ of the protospacer. Cas12a enzymes recognize T-rich PAMs that are positioned 5′ to the spacer sequence. Cas12a has been reported to recognize a PAM of the form TTTN but strongly prefers TTTV (where V=A, C, or G). Cas12a can be programmed to edit target sites in human cells and possess robust on-target activities and high genome-wide specificities in human cells (see, e.g., Kleinstiver et al., Nat Biotechnol. (2016) 34(8):869-74. PMID: 27347757; and Kim et al., Nat Biotechnol. (2016) 34(8):863-8. PMID: 27272384; Zetsche et al., Cell (2015) 163, 759-771. PMID: 26422227; Shmakov et al., Mol Cell. (2015) 60(3): 385-97. PMID: 26593719). In some embodiments, it is of interest to make use of an engineered Cas12a domain as defined herein, such as Cas12a, wherein the Cas12a domain complexes with a nucleic acid molecule including RNA to form a CRISPR complex, wherein when in the CRISPR complex, the nucleic acid molecule targets one or more target polynucleotide loci. In certain embodiments, the Cas12a domain is modified to have increased activity, i.e. wider PAM specificity.

In some embodiments, the present disclosure relates to Cas12a domains. The term “Cas12a” or “Cas12a nuclease” refers to an RNA-guided nuclease including a Cas12a protein, or portion thereof (e.g., a protein including an active, inactive, or partially active DNA cleavage domain of Cas12a, and/or the gRNA binding domain of Cas12a). A Cas12a nuclease may also be referred to as a Cpf1 nuclease. Cas12a orthologs have been described in various species, including, but not limited to, Lachnospiraceae bacterium (LbCpf1) nuclease and Acidaminococcus sp. (AsCpf1). A nuclease-inactivated Cas12a domain, or deactivated Cas12a domain, may be referred to as a “dCas12a” domain (for nuclease “dead” Cas12a). A Cas12a variant capable of generating a single-strand DNA break (nick) may be referred to as “Cas12a nickase”, “nCas12a”, “Cpf1 nickase”, or “nCpf1.” Methods for generating a Cas12a protein, or a portion thereof, having an inactive DNA cleavage domain are known in the art. Additional suitable Cas12a nucleases and sequences will be apparent to those of skill in the art based on this disclosure.

In some embodiments, the present disclosure relates to Cas12a domains. In some embodiments, the Cas12a domain includes an amino acid sequence that is at least about 60% identical, at least about 65% identical, at least about 70% identical, at least about 72.5% identical, at least about 75% identical, at least about 77.5% identical, at least about 80% identical, at least about 82.5% identical, at least about 85% identical, at least about 87.5% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, at least about 99.5% identical, or at least about 99.9% identical to any one of SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413. In some embodiments, the Cas12a domain is SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413. In some embodiments, the Cas12a domain includes an amino acid sequence that has 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more mutations compared to any one of the amino acid sequences set forth in SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413. In some embodiments, the Cas12a domain includes an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth in SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413. Additional suitable Cas12a domains (e.g., from Acidaminococcus sp., Lachnospiraceae sp., or other species) will be apparent to those of skill in the art based on this disclosure and knowledge in the field, and are within the scope of this disclosure.

In some embodiments, the Cas12a domain of any of the base-editing systems provided herein is a dCas12a domain. For example, a dCas12a domain may be produced by inactivating the RuvC-like domain of Cas12a, as described in Zetsche et al., Cell (2015) 163, 759-771. PMID: 26422227. For example, mutations corresponding to D917A, E1006A, E1028A, D1227A, D1255A, or N1257A in Francisella novicida Cas12a (SEQ ID NO: 356) inactivate Cas12a nuclease activity. In some embodiments, the dCas12a includes mutations corresponding to D917A, E1006A, D1255A, D917A/E1006A, D917A/D1255A, E1006A/D1255A, or D917A/E1006A/D1255A in SEQ ID NO: 350. In some embodiments, the inactivated Cas12a domains include enzymes mutated in amino acid positions 908, 993, 1263 of AsCas12a or corresponding positions in Cas12a orthologs. More particularly, the inactivated Cas12a domains include domains including one or more of mutations D908A, E993A, D1263A of AsCas12a or corresponding mutations in Cas12a orthologs. In some embodiments, the inactivated Cas12a domains include enzymes mutated in amino acid position 832, 925, 947 or 1180 of LbCas12a or corresponding positions in Cas12a orthologs. More particularly, the inactivated Cas12a domains include domains including one or more of mutations D832A, E925A, D947A or D1180A of LbCas12a or corresponding mutations in Cas12a orthologs. Mutations can also be made at neighboring residues, e.g., at amino acids near those indicated above that participate in the nuclease activity. In some embodiments, only the RuvC domain is inactivated, while in other embodiments, another putative nuclease domain is inactivated, wherein the effector protein complex functions as a nickase and cleaves only one DNA strand. In some embodiments, the dCas12a is a nuclease inactive SEQ ID NO: 359. In some embodiments, the dCas12a includes a D832X mutation relative to SEQ ID NO: 359, wherein X is any amino acid except for D. In some embodiments, the dCas12a includes a D832A mutation relative to SEQ ID NO: 359. Additional dCas12a proteins have been described (see, e.g., Li et al., Nat. Biotech. (2018) DOI: 10.1038/nbt.4102). In some embodiments, the dCas12a includes 1, 2, or 3 of the point mutations D832A, E1006A, D1125A of the Cas12a described in Li et al., Nat. Biotech. (2018) DOI: 10.1038/nbt.4102. In some embodiments, dCas12a corresponds to, in part or in whole, a Cas12a amino acid sequence having one or more mutations that inactivate the Cas12a nuclease activity. Additional suitable dCas12a domains will be apparent to those of skill in the art based on this disclosure and knowledge in the field, and are within the scope of this disclosure. In some embodiments, the dCas12a domain includes an amino acid sequence that is at least 60% identical, at least 65% identical, at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to any one of the dCas12a domains provided herein. It is to be understood that any mutations, (e.g., substitution mutations, deletions, insertions, etc.) that inactivate the RuvC domain of Cas12a, may be used in accordance with the present disclosure.

In some embodiments, the Cas12a domain of any of the base-editing systems provided herein is a Cas12a nickase domain. In some embodiments, the Cas12a nickase includes a R836X mutation relative to SEQ ID NO: 359, wherein X is any amino acid except for R. In some embodiments, the Cas12a nickase includes a R836A mutation relative to SEQ ID NO: 359. In some embodiments, the Cas12a nickase includes a R1138A mutation relative to SEQ ID NO: 359. In some embodiments, the Cas12a nickase includes a R912A mutation relative to SEQ ID NO: 358. Without wishing to be bound by any particular theory, residue R836 of SEQ ID NO: 359 and residue R912 of SEQ ID NO: 358 are examples of corresponding (e.g., homologous) residues. In some embodiments, the Cas12a nickase includes a R1226A mutation relative to SEQ ID NO: 350 (AsCas12a). In some embodiments, the Cas12a nickase includes a mutation at residues R1218 relative to SEQ ID NO: 390 (FnCas12a). In some embodiments, the Cas12a nickase includes a mutation at residue R1293 relative to SEQ ID NO: 400 (MbCas12a). It will be understood by the skilled person that a mutation may be made at a residue in a corresponding position to the positions described herein in a Cas12a ortholog. In some embodiments, the Cas12a nickase may be a Cas12a protein that is capable of cleaving only one strand of a duplexed nucleic acid molecule (e.g., a duplexed DNA molecule). In some embodiments the Cas12a nickase cleaves the target strand of a duplexed nucleic acid molecule, meaning that the Cas12a nickase cleaves the strand that is base paired to (complementary to) a gRNA (e.g., an sgRNA) that is bound to the Cas12a. In some embodiments the Cas12a nickase includes an amino acid sequence that is at least 60% identical, at least 65% identical, at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to any one of the Cas12a nickases provided herein. In some embodiments, any of the Cas12a proteins provided herein includes one or more amino acid deletions. Additional suitable Cas12a nickases will be apparent to those of skill in the art based on this disclosure and knowledge in the field, and are within the scope of this disclosure. In some embodiments, the inactivated Cas12a or Cas12a nickase may have associated (e.g., via fusion protein) one or more functional domains, including for example, a cytidine deaminase or catalytic domain thereof. In some embodiments, the Cas12a nickase includes a Cas12a nickase that cleaves the non-target strand, as described in Yamano et al., Cell (2016) 165(4): 949-962. PMID: 27114038. Without wishing to be bound by any particular theory, Cas12a nickase cuts the target strand more efficiently than the non-target strand.

In some embodiments, the Cas12a domain of any of the base-editing systems provided herein is a modified Cas12a domain. Several small stretches of unstructured regions are predicted within the Cas12a primary structure. Without wishing to be bound by theory, unstructured regions, which are exposed to the solvent and not conserved within different Cas12a orthologs, are preferred sites for splits and insertions of small protein sequences. In addition, these sites can be used to generate chimeric proteins between Cas12a orthologs. Based on the above information, mutants can be generated which lead to inactivation of the Cas12a or which modify the double strand nuclease to nickase activity. In some embodiments, this information is used to develop Cas12a domains with reduced off-target effects. In some embodiments, any of the Cas12a domains described herein is modified by mutation of one or more residues (in the RuvC domain) including but not limited to positions R909, R912, R930, R947, K949, R951, R955, K965, K968, K1000, K1002, R1003, K1009, K1017, K1022, K1029, K1035, K1054, K1072, K1086, R1094, K1095, K1109, K1118, K1142, K1150, K1158, K1159, R1220, R1226, R1242, and/or R1252 with reference to amino acid position numbering of SEQ ID NO: 396 (AsCas12a from Acidaminococcus sp. BV3L6). In some embodiments, the Cas12a domains including said one or more mutations have modified, more preferably increased specificity for the target. In some embodiments, any of the Cas12a domains described herein is modified by mutation of one or more residues (in the RAD50) domain including but not limited to positions K324, K335, K337, R331, K369, K370, R386, R392, R393, K400, K404, K406, K408, K414, K429, K436, K438, K459, K460, K464, R670, K675, R681, K686, K689, R699, K705, R725, K729, K739, K748, and/or K752 with reference to amino acid position numbering of SEQ ID NO: 396 (AsCas12a from Acidaminococcus sp. BV3L6). In some embodiments, the Cas12a domains including said one or more mutations have modified, more preferably increased specificity for the target. In certain of the Cas12a domains, the enzyme is modified by mutation of one or more residues including but not limited to positions R912, T923, R947, K949, R951, R955, K965, K968, K1000, R1003, K1009, K1017, K1022, K1029, K1072, K1086, F1103, R1226, and/or R1252 with reference to amino acid position numbering of AsCas12a (Acidaminococcus sp. BV3L6). In certain embodiments, the Cas12a domains including said one or more mutations have modified, more preferably increased specificity for the target. In some embodiments, any of the Cas12a domains described herein is modified by mutation of one or more residues including but not limited to positions R833, R836, K847, K879, K881, R883, R887, K897, K900, K932, R935, K940, K948, K953, K960, K984, K1003, K1017, R1033, R1138, R1165, and/or R1252 with reference to amino acid position numbering of SEQ ID NO: 359 (LbCas12a from Lachnospiraceae bacterium ND2006). In certain embodiments, the Cas12a domains including said one or more mutations have modified, more preferably increased specificity for the target. Additional Cas12a mutations may be found in WO2018213726.

In some embodiments, proteins including portions of Cas12a are provided. In some embodiments, proteins including Cas12a or portions thereof are referred to as “Cas12a variants.” A Cas12a variant shares homology to Cas12a, or a portion thereof. For example, a Cas12a variant is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to wild type Cas12a. In some embodiments, the Cas12a variant may have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more amino acid changes compared to wild type Cas12a. In some embodiments, the Cas12a variant includes a portion of Cas12a (e.g., a gRNA binding domain or a DNA-cleavage domain), such that the Cas12a portion is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the corresponding portion of wild type Cas12a. In some embodiments, the Cas12a portion is at least 50%, at least 55%, at least 60%, at least 65%, at least 70%, at least 72.5%, at least 75%, at least 77.5%, at least 80%, at least 82.5%, at least 85%, at least 87.5%, at least 90%, at least 91%, at least 92%, at least 93%, at least 94%, at least 95%, at least 96%, at least 97%, at least 98%, at least 99%, at least 99.5%, or at least 99.9% of the amino acid length of a corresponding wild type Cas12a. In some embodiments, the Cas12a portion is at least 100 amino acids in length. In some embodiments, the Cas12a portion is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 amino acids in length.

In some embodiments, the Cas12a is derived from a bacterial species selected from Francisella tularensis 1, Prevotella albensis, Lachnospiraceae bacterium MC2017, Lachnospiraceae bacterium MA2020, Lachnospiraceae bacterium ND2006, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Candidatus Methanoplasma termitum, Eubacterium eligens, Moraxella bovoculi 237, Moraxella bovoculi AAX08_00205, Moraxella bovoculi AAX11_00205, Butyrivibrio sp. NC3005, Thiomicrospira sp. XS5, Leptospira inadai, Porphyromonas crevioricanis 3, Prevotella disiens and Porphyromonas macacae. In some embodiments, the Cas12a is derived from a subspecies of Francisella tularensis 1, including but not limited to Francisella tularensis subsp. novicida. In some embodiments, the Cas12a is derived from a bacterial species selected from Acidaminococcus sp. BV3L6, Lachnospiraceae bacterium ND2006, Lachnospiraceae bacterium MA2020, Moraxella bovoculi AAX08_00205, Moraxella bovoculi AAX11_00205, Butyrivibrio sp. NC3005, or Thiomicrospira sp. XS5. In some embodiments, wild type Cas12a corresponds to Cas12a from Lachnospiraceae bacterium (e.g., Lachnospiraceae bacterium ND2006, MC2017, or MA2020), i.e., SEQ ID NO: 359, SEQ ID NO: 391, or SEQ ID NO: 397. In some embodiments, wild type Cas12a corresponds to Cas12a from Acidaminococcus sp., i.e., SEQ ID NO: 350. In some embodiments, wild type Cas12a corresponds to Cas12a from Butyrivibrio proteoclasticus (BpCas12a), i.e., SEQ ID NO: 392. In some embodiments, wild type Cas12a corresponds to Cas12a from Peregrinibacteria bacterium GW2011_GWA_33_10 (PeCas12a), i.e., SEQ ID NO: 393. In some embodiments, wild type Cas12a corresponds to Cas12a from Parcubacteria bacterium GWC2011_GWC2_44_17 (PbCas12a), i.e., SEQ ID NO: 394. In some embodiments, wild type Cas12a corresponds to Cas12a from Smithella sp. SC_K08D17 (SsCas12a), i.e., SEQ ID NO: 395. In some embodiments, wild type Cas12a corresponds to Cas12a from Acidaminococcus sp. BV3L6 (AsCas12a), i.e., SEQ ID NO: 396. In some embodiments, wild type Cas12a corresponds to Cas12a from Lachnospiraceae bacterium MVA2020 (Lb2Cas12a), i.e., SEQ ID NO: 397. In some embodiments, wild type Cas12a corresponds to Cas12a from Candidatus methanoplasma termitum (CMtCas12a), i.e., SEQ ID NO: 398. In some embodiments, wild type Cas12a corresponds to Cas12a from Eubacterium eligens (EeCas12a), i.e., SEQ ID NO: 399. In some embodiments, wild type Cas12a corresponds to Cas12a from Moraxella bovoculi 237 (MbCas12a), i.e., SEQ ID NO: 400. In some embodiments, wild type Cas12a corresponds to Cas12a from Leptospira inadai (LiCas12a), i.e., SEQ ID NO: 401. In some embodiments, wild type Cas12a corresponds to Cas12a from Porphyromonas crevioricanis (PcCas12a), i.e., SEQ ID NO: 403. In some embodiments, wild type Cas12a corresponds to Cas12a from Prevotella disiens (PdCas12a), i.e., SEQ ID NO: 404. In some embodiments, wild type Cas12a corresponds to Cas12a from Porphyromonas macacae (PmCas12a), i.e., SEQ ID NO: 405. In some embodiments, wild type Cas12a corresponds to Cas12a from Thiomicrospira sp. XS5 (TsCas12a), i.e., SEQ ID NO: 406. In some embodiments, wild type Cas12a corresponds to Cas12a from Moraxella bovoculi AAX08_00205 (Mb2Cas12a), i.e., SEQ ID NO: 407. In some embodiments, wild type Cas12a corresponds to Cas12a from Moraxella bovoculi AAX11_00205 (Mb3Cas12a), i.e., SEQ ID NO: 408. In some embodiments, wild type Cas12a corresponds to Cas12a from Butyrivibrio sp. NC3005 (BsCas12a), i.e., SEQ ID NO: 409. In some embodiments, Cas12a corresponds to Cas12a ortholog NCBI WP_055225123.1, i.e., SEQ ID NO: 410. In some embodiments, Cas12a corresponds to Cas12a ortholog NCBI WP_055237260.1, 1. e., SEQ ID NO: 411. In some embodiments, Cas12a corresponds to Cas12a ortholog NCBI WP_055272206.1, i.e., SEQ ID NO: 412. In some embodiments, Cas12a corresponds to Cas12a ortholog GenBank OLA16049.1, i.e., SEQ ID NO: 413.

In some embodiments, Cas12a corresponds to a codon-optimized Cas12a from any one of the species herein. Codon-optimized Cas12a sequences may be used when the fusion protein of the present disclosure is administered as a nucleic acid. An example of a codon-optimized sequence, is for example, a sequence optimized for expression in a eukaryote (e.g., a mammal such as a human, a plant such as corn, etc.). Information regarding codon usage in different organisms is readily available. Similarly, there are a variety of tools that may be used (e.g., codon usage tables, algorithms, programs, etc.) for codon-optimizing sequences.

In some embodiments, base-editing systems as provided herein include the full-length amino acid sequence of a Cas12a protein, e.g., one of the Cas12a sequences provided herein. In other embodiments, however, base-editing systems as provided herein do not include a full-length Cas12a sequence, but only a portion thereof. For example, in some embodiments, a base-editing system provided herein includes a portion of a Cas12a protein, wherein the portion binds crRNA and tracrRNA or sgRNA, but does not include a functional nuclease domain, e.g., in that it includes only a truncated version of a nuclease domain or no nuclease domain at all. In some embodiments, any of the Cas12a proteins provided herein includes 1, 2, 3, 4, 5, 6, 7, 8, 9, 10, 11, 12, 13, 14, 15, 16, 17, 18, 19, or 20 amino acid deletions. Without wishing to be bound by any particular theory, there is a helical region in Cas12a, which includes residues 661-667 of SEQ ID NO: 358, that may obstruct the function of a cytidine deaminase that is fused to the Cas12a. Accordingly, aspects of the disclosure provide Cas12a proteins including mutations (e.g., deletions) that disrupt this helical region in Cas12a. In some embodiments, the Cas12a protein includes one or more deletions of the residues K661, K662, T663, G664, D665, Q666, and K667 in SEQ ID NO: 358, or one or more corresponding deletions in another Cas12a protein.

In some embodiments, a base-editing system provided herein includes a “split Cas12a” protein. For example, split Cas12a proteins which have N- and C-termini that differ from wild-type Cas12a proteins. Split Cas12a proteins offer different points of fusion for a deaminase. For example, a deaminase could be integrated at the split point of a split Cas12a protein. See, Nihongaki et al., Nat Chem Biol. (2019) (9):882-888. PMID: 31406371. In other embodiments, a base-editing system provided herein includes Cas12a proteins with novel protein topologies. In some embodiments, the Cas12a protein domain is reorganized but no truncated, such as in ‘circular permutation’ of proteins. For example, in some embodiments, Cas12a protein is circularized via simultaneous fusion of a deaminase to both N- and C-termini, to create new N- and C-termini at another position in the sequence, in effect, moving a C-terminal portion of the protein to the N-terminus. See, Yu et al., Trends Biotechnol. (2011) 29(1): 18-25. PMID: 21087800. Exemplary amino acid sequences of suitable Cas12a domains and portions thereof are provided herein, and additional suitable sequences of Cas12a domains and portions thereof will be apparent to those of skill in the art.

Some aspects of the disclosure provide base-editing systems (e.g., any of the base-editing systems provided herein) including a Cas12a domain that has high fidelity. Additional aspects of the disclosure provide Cas12a base-editing systems (e.g., any of the base-editing systems provided herein) including a Cas12a domain with decreased electrostatic interactions between the Cas12a domain and a sugar-phosphate backbone of a DNA, as compared to a wild-type Cas12a domain. In some embodiments, a Cas12a domain (e.g., a wild type Cas12a domain) includes one or more mutations that decreases the association between the Cas12a domain and a sugar-phosphate backbone of a DNA. Cas12a domains with high fidelity are known in the art and would be apparent to the skilled artisan.

### Cas12h and Cas12i Domains

In some embodiments, the DNA binding domain of any of the base-editing systems provided herein is a Cas12h or a Cas12i. Cas12h and Cas12i demonstrate RNA-guided double-stranded (dsDNA) interference activity. Further, Cas12i exhibits markedly different efficiencies of CRISPR RNA spacer complementary and noncomplementary strand cleavage resulting in predominant dsDNA nicking (see, e.g., Yan et al., Science (2019) 363(6422):88-91. PMID: 30523077). In some embodiments, Cas refers to a Cas12h or a Cas12i. Suitable Cas12h domains and Cas12i domains will be apparent to those of skill in the art based on this disclosure and knowledge in the field, and are within the scope of this disclosure (see, e.g., Yan et al., Science (2019) 363, Issue 6422, pp. 88-91. PMID: 30523077).

In some embodiments, the present disclosure relates to nuclease inactive Cas12h domains (dCas12h) or Cas12h nickase domains. In some embodiments, the present disclosure relates to nuclease inactive Cas12i domains (dCas12i) or Cas12i nickase domains.

In some embodiments, proteins including portions of Cas12h are provided. In some embodiments, proteins including Cas12h or portions thereof are referred to as “Cas12h variants.” A Cas12h variant shares homology to Cas12h, or a portion thereof. For example, a Cas12h variant is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to wild type Cas12h. In some embodiments, the Cas12h variant may have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more amino acid changes compared to wild type Cas12h. In some embodiments, the Cas12h variant includes a portion of Cas12h (e.g., a gRNA binding domain or a DNA-cleavage domain), such that the Cas12h portion is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the corresponding portion of wild type Cas12h. In some embodiments, the Cas12h portion is at least 50%, at least 55%, at least 60%, at least 65%, at least 70%, at least 72.5%, at least 75%, at least 77.5%, at least 80%, at least 82.5%, at least 85%, at least 87.5%, at least 90%, at least 91%, at least 92%, at least 93%, at least 94%, at least 95%, at least 96%, at least 97%, at least 98%, at least 99%, at least 99.5%, or at least 99.9% of the amino acid length of a corresponding wild type Cas12h. In some embodiments, the Cas12h portion is at least 100 amino acids in length. In some embodiments, the Cas12h portion is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 amino acids in length.

In some embodiments, proteins including portions of Cas12i are provided. In some embodiments, proteins including Cas12i or portions thereof are referred to as “Cas12i variants.” A Cas12i variant shares homology to Cas12i, or a portion thereof. For example, a Cas12i variant is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to wild type Cas12i. In some embodiments, the Cas12i variant may have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more amino acid changes compared to wild type Cas12i. In some embodiments, the Cas12i variant includes a portion of Cas12i (e.g., a gRNA binding domain or a DNA-cleavage domain), such that the Cas12i portion is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the corresponding portion of wild type Cas12i. In some embodiments, the Cas12i portion is at least 50%, at least 55%, at least 60%, at least 65%, at least 70%, at least 72.5%, at least 75%, at least 77.5%, at least 80%, at least 82.5%, at least 85%, at least 87.5%, at least 90%, at least 91%, at least 92%, at least 93%, at least 94%, at least 95%, at least 96%, at least 97%, at least 98%, at least 99%, at least 99.5%, or at least 99.9% of the amino acid length of a corresponding wild type Cas12i. In some embodiments, the Cas12i portion is at least 100 amino acids in length. In some embodiments, the Cas12i portion is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 amino acids in length.

### CasX and CasY Domains

In some embodiments, the DNA binding domain of any of the base-editing systems provided herein is a Cas domain from archaea (e.g., nanoarchaea), which constitute a domain and kingdom of single-celled prokaryotic microbes. In some embodiments, the DNA binding domain is CasX or CasY, which have been described in, for example, Burstein et al., Cell Res (2017) 542(7640):237-241. PMID: 28005056. Using genome-resolved metagenomics, a number of CRISPR-Cas systems were identified, including the first reported Cas domain in the archaeal domain of life. This divergent Cas protein was found in nanoarchaea as part of an active CRISPR-Cas system. In bacteria, two previously unknown systems were discovered, CRISPR-CasX and CRISPR-CasY, which are among the most compact systems yet discovered.

In some embodiments, Cas refers to CasX, or a variant of CasX. In some embodiments, Cas refers to a CasY, or a variant of CasY. Additional suitable CasX domains and CasY domains will be apparent to those of skill in the art based on this disclosure and knowledge in the field, and are within the scope of this disclosure.

In some embodiments, the DNA binding domain of any of the base-editing systems provided herein is a CasX domain. In some embodiments, the CasX domain includes an amino acid sequence that is that is at least about 60% identical, at least about 65% identical, at least about 70% identical, at least about 72.5% identical, at least about 75% identical, at least about 77.5% identical, at least about 80% identical, at least about 82.5% identical, at least about 85% identical, at least about 87.5% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, at least about 99.5% identical, or at least about 99.9% identical to SEQ ID NO: 370 or SEQ ID NO: 371. In some embodiments, the CasX domain is SEQ ID NO: 370 or SEQ ID NO: 371. In some embodiments, the CasX domain includes an amino acid sequence that has 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more mutations compared to any one of the amino acid sequences set forth in SEQ ID NO: 370 or SEQ ID NO: 371. In some embodiments, the CasX domain includes an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth in SEQ ID NO: 370 or SEQ ID NO: 371. In some embodiments, the present disclosure relates to nuclease inactive CasX domains (dCasX) or CasX nickase domains.

In some embodiments, proteins including portions of CasX are provided. In some embodiments, proteins including CasX or portions thereof are referred to as “CasX variants.” A CasX variant shares homology to CasX, or a portion thereof. For example, a CasX variant is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to wild type CasX. In some embodiments, the CasX variant may have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more amino acid changes compared to wild type CasX. In some embodiments, the CasX variant includes a portion of CasX (e.g., a gRNA binding domain or a DNA-cleavage domain), such that the CasX portion is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the corresponding portion of wild type CasX. In some embodiments, the CasX portion is at least 50%, at least 55%, at least 60%, at least 65%, at least 70%, at least 72.5%, at least 75%, at least 77.5%, at least 80%, at least 82.5%, at least 85%, at least 87.5%, at least 90%, at least 91%, at least 92%, at least 93%, at least 94%, at least 95%, at least 96%, at least 97%, at least 98%, at least 99%, at least 99.5%, or at least 99.9% of the amino acid length of a corresponding wild type CasX. In some embodiments, the CasX portion is at least 100 amino acids in length. In some embodiments, the CasX portion is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 amino acids in length. Additional suitable CasX domains (e.g., from other bacterial species) will be apparent to those of skill in the art based on this disclosure and knowledge in the field, and are within the scope of this disclosure.

In some embodiments, the DNA binding domain of any of the base-editing systems provided herein is a CasY domain. In some embodiments, the CasY domain includes an amino acid sequence that is that is at least about 60% identical, at least about 65% identical, at least about 70% identical, at least about 72.5% identical, at least about 75% identical, at least about 77.5% identical, at least about 80% identical, at least about 82.5% identical, at least about 85% identical, at least about 87.5% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, at least about 99.5% identical, or at least about 99.9% identical to SEQ ID NO: 372. In some embodiments, the CasY domain is SEQ ID NO: 372. In some embodiments, the CasY domain includes an amino acid sequence that has 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more mutations compared to any one of the amino acid sequences set forth in SEQ ID NO: 372. In some embodiments, the CasY domain includes an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth in SEQ ID NO: 372. Additional suitable CasY domains will be apparent to those of skill in the art based on this disclosure and knowledge in the field, and are within the scope of this disclosure. In some embodiments, the present disclosure relates to nuclease inactive CasY domains (dCasY) or CasY nickase domains.

In some embodiments, proteins including portions of CasY are provided. In some embodiments, proteins including CasY or portions thereof are referred to as “CasY variants.” A CasY variant shares homology to CasY, or a portion thereof. For example, a CasY variant is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to wild type CasY. In some embodiments, the CasY variant may have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more amino acid changes compared to wild type CasY. In some embodiments, the CasY variant includes a portion of CasY (e.g., a gRNA binding domain or a DNA-cleavage domain), such that the CasY portion is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the corresponding portion of wild type CasY. In some embodiments, the CasY portion is at least 50%, at least 55%, at least 60%, at least 65%, at least 70%, at least 72.5%, at least 75%, at least 77.5%, at least 80%, at least 82.5%, at least 85%, at least 87.5%, at least 90%, at least 91%, at least 92%, at least 93%, at least 94%, at least 95%, at least 96%, at least 97%, at least 98%, at least 99%, at least 99.5%, or at least 99.9% of the amino acid length of a corresponding wild type CasY. In some embodiments, the CasY portion is at least 100 amino acids in length. In some embodiments, the CasY portion is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 amino acids in length. Additional suitable CasY domains (e.g., from other bacterial species) will be apparent to those of skill in the art based on this disclosure and knowledge in the field, and are within the scope of this disclosure.

### C2c1, C2c2, and C2c3 Domains

In addition to Cas9 and Cas12a, three distinct Class 2 CRISPR-Cas systems (C2c1, C2c2, and C2c3) have been described by Shmakov et al., Mol. Cell (2015) 60(3): 385-397. PMID: 26593719. Effectors of two of the systems, C2c1 and C2c3, contain RuvC-like endonuclease domains related to Cas12a. A third system, C2c2 contains an effector with two predicted HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cas12a (see, e.g., East-Seletsky, et al., Nature (2016) 538(7624):270-273. PMID: 27669025). In vitro biochemical analysis of C2c2 in Leptotrichia shahii has shown that C2c2 is guided by a single CRISPR RNA and can be programmed to cleave ssRNA targets carrying complementary protospacers. Catalytic residues in the two conserved HEPN domains mediate cleavage. Mutations in the catalytic residues generate catalytically inactive RNA-binding proteins (see, e.g., Abudayyeh et al., Science (2016) 353(6299). PMID: 27256883).

The crystal structure of Alicyclobaccillus acidoterrastris C2c1 (AacC2c1) has been reported in complex with a chimeric single-molecule guide RNA (sgRNA) (see, e.g., Liu et al., Mol. Cell (2017) 65(2):310-322. PMID: 27989439). The crystal structure has also been reported for A. acidoterrestris C2c1 bound to target DNAs as ternary complexes (see, e.g., Yang et al., Cell (2016) 167(7): 1814-1828. PMID: 27984729). Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cas12a counterparts demonstrate the diversity of mechanisms used by CRISPR-Cas9 systems. Additional suitable C2c1 domains, C2c2 domains, and C2c3 domains will be apparent to those of skill in the art based on this disclosure and knowledge in the field, and are within the scope of this disclosure.

In some embodiments, the DNA binding domain of any of the base-editing systems provided herein is a C2c1 domain. In some embodiments, the C2c1 domain includes an amino acid sequence that is that is at least about 60% identical, at least about 65% identical, at least about 70% identical, at least about 72.5% identical, at least about 75% identical, at least about 77.5% identical, at least about 80% identical, at least about 82.5% identical, at least about 85% identical, at least about 87.5% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, at least about 99.5% identical, or at least about 99.9% identical to SEQ ID NO: 367. In some embodiments, the C2c1 domain is SEQ ID NO: 367. In some embodiments, the C2c1 domain includes an amino acid sequence that has 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more mutations compared to any one of the amino acid sequences set forth in SEQ ID NO: 367. In some embodiments, the C2c1 domain includes an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth in SEQ ID NO: 367. In some embodiments, the present disclosure relates to nuclease inactive C2c1 domains (dC2c1) or C2c1 nickase domains.

In some embodiments, proteins including portions of C2c1 are provided. In some embodiments, proteins including C2c1 or portions thereof are referred to as “C2c1 variants.” A C2c1 variant shares homology to C2c1, or a portion thereof. For example, a C2c1 variant is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to wild type C2c1. In some embodiments, the C2c1 variant may have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more amino acid changes compared to wild type C2c1. In some embodiments, the C2c1 variant includes a portion of C2c1 (e.g., a gRNA binding domain or a DNA-cleavage domain), such that the C2c1 portion is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the corresponding portion of wild type C2c1. In some embodiments, the C2c1 portion is at least 50%, at least 55%, at least 60%, at least 65%, at least 70%, at least 72.5%, at least 75%, at least 77.5%, at least 80%, at least 82.5%, at least 85%, at least 87.5%, at least 90%, at least 91%, at least 92%, at least 93%, at least 94%, at least 95%, at least 96%, at least 97%, at least 98%, at least 99%, at least 99.5%, or at least 99.9% of the amino acid length of a corresponding wild type C2c1. In some embodiments, the C2c1 portion is at least 100 amino acids in length. In some embodiments, the C2c1 portion is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 amino acids in length. Additional suitable C2c1 domains (e.g., from other bacterial species) will be apparent to those of skill in the art based on this disclosure and knowledge in the field, and are within the scope of this disclosure.

In some embodiments, the DNA binding domain of any of the base-editing systems provided herein is a C2c2 domain. In some embodiments, the C2c2 domain includes an amino acid sequence that is that is at least about 60% identical, at least about 65% identical, at least about 70% identical, at least about 72.5% identical, at least about 75% identical, at least about 77.5% identical, at least about 80% identical, at least about 82.5% identical, at least about 85% identical, at least about 87.5% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, at least about 99.5% identical, or at least about 99.9% identical to SEQ ID NO: 368. In some embodiments, the C2c2 domain is SEQ ID NO: 368. In some embodiments, the C2c2 domain includes an amino acid sequence that has 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more mutations compared to any one of the amino acid sequences set forth in SEQ ID NO: 368. In some embodiments, the C2c2 domain includes an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth in SEQ ID NO: 368. In some embodiments, the present disclosure relates to nuclease inactive C2c2 domains (dC2c2) or C2c2 nickase domains.

In some embodiments, proteins including portions of C2c2 are provided. In some embodiments, proteins including C2c2 or portions thereof are referred to as “C2c2 variants.” A C2c2 variant shares homology to C2c2, or a portion thereof. For example, a C2c2 variant is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to wild type C2c2. In some embodiments, the C2c2 variant may have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more amino acid changes compared to wild type C2c2. In some embodiments, the C2c2 variant includes a portion of C2c2 (e.g., a gRNA binding domain or a DNA-cleavage domain), such that the C2c2 portion is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the corresponding portion of wild type C2c2. In some embodiments, the C2c2 portion is at least 50%, at least 55%, at least 60%, at least 65%, at least 70%, at least 72.5%, at least 75%, at least 77.5%, at least 80%, at least 82.5%, at least 85%, at least 87.5%, at least 90%, at least 91%, at least 92%, at least 93%, at least 94%, at least 95%, at least 96%, at least 97%, at least 98%, at least 99%, at least 99.5%, or at least 99.9% of the amino acid length of a corresponding wild type C2c2. In some embodiments, the C2c2 portion is at least 100 amino acids in length. In some embodiments, the C2c2 portion is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 amino acids in length. Additional suitable C2c2 domains (e.g., from other bacterial species) will be apparent to those of skill in the art based on this disclosure and knowledge in the field, and are within the scope of this disclosure.

In some embodiments, the DNA binding domain of any of the base-editing systems provided herein is a C2c3 domain. In some embodiments, the C2c3 domain includes an amino acid sequence that is that is at least about 60% identical, at least about 65% identical, at least about 70% identical, at least about 72.5% identical, at least about 75% identical, at least about 77.5% identical, at least about 80% identical, at least about 82.5% identical, at least about 85% identical, at least about 87.5% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, at least about 99.5% identical, or at least about 99.9% identical to SEQ ID NO: 369. In some embodiments, the C2c3 domain is SEQ ID NO: 369. In some embodiments, the C2c3 domain includes an amino acid sequence that has 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more mutations compared to any one of the amino acid sequences set forth in SEQ ID NO: 369. In some embodiments, the C2c3 domain includes an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth in SEQ ID NO: 369. In some embodiments, the present disclosure relates to nuclease inactive C2c3 domains (dC2c3) or C2c3 nickase domains. [M111] In some embodiments, proteins including portions of C2c3 are provided. In some embodiments, proteins including C2c3 or portions thereof are referred to as “C2c3 variants.” A C2c3 variant shares homology to C2c3, or a portion thereof. For example, a C2c3 variant is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to wild type C2c3. In some embodiments, the C2c3 variant may have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more amino acid changes compared to wild type C2c3. In some embodiments, the C2c3 variant includes a portion of C2c3 (e.g., a gRNA binding domain or a DNA-cleavage domain), such that the C2c3 portion is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the corresponding portion of wild type C2c3. In some embodiments, the C2c3 portion is at least 50%, at least 55%, at least 60%, at least 65%, at least 70%, at least 72.5%, at least 75%, at least 77.5%, at least 80%, at least 82.5%, at least 85%, at least 87.5%, at least 90%, at least 91%, at least 92%, at least 93%, at least 94%, at least 95%, at least 96%, at least 97%, at least 98%, at least 99%, at least 99.5%, or at least 99.9% of the amino acid length of a corresponding wild type C2c3. In some embodiments, the C2c3 portion is at least 100 amino acids in length. In some embodiments, the C2c3 portion is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 amino acids in length. Additional suitable C2c3 domains (e.g., from other bacterial species) will be apparent to those of skill in the art based on this disclosure and knowledge in the field, and are within the scope of this disclosure.

### TALE Domains

Transcription activator-like effectors (TALEs) can be engineered to bind practically any DNA sequence. TALEs are DNA binding domains derived from various plant bacterial pathogens of the genus Xanthomonas, which secrete TALEs into the host plant cell during infection. The TALEs move to the nucleus, where they recognize and bind to a specific DNA sequence in the promoter region of a specific gene in the host genome. TALEs have a central DNA binding domain composed of 13-28 repeat monomers of 33-34 amino acids. The amino acids of each monomer are highly conserved, except for hypervariable amino acid residues at positions 12 and 13. The two hypervariable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs. The transcription activator-like effector (TALE) DNA binding domain can be fused to a functional domain, such as a recombinase, a nuclease, a transposase or a helicase, thus conferring sequence specificity to the functional domain.

The TALE disclosed herein, may be full-length or a portion thereof. In some embodiments, proteins including TALE, or portions thereof, are referred to as “TALE variants”. A TALE variant shares homology to TALE, or a portion thereof. For example a TALE variant is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to wild type TALE. In some embodiments, the TALE variant includes a portion of TALE, such that the portion is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the corresponding portion of wild type TALE.

The relationship between amino acid sequence and DNA recognition of the TALE binding domain allows for designable proteins. Software programs such as DNA Works can be used to design TALE constructs. Other methods of designing TALE constructs are known to those of skill in the art (see, e.g., Doyle et al., Nucleic Acids Res (2012) 40(W1):W117- W122; and Cermak, Nucleic Acids Res (2011) 39(12):e82).

### Zinc Finger Domains

Zinc fingers (ZFs) are engineered DNA binding domains. Recognition site specificity is conferred by the zinc finger domain, which typically includes two, three, or four zinc fingers, for example having a C2H2 structure, however other zinc finger structures are known and have been engineered. Zinc finger domains can be used for designing polypeptides which specifically bind a selected polynucleotide recognition sequence. Additional functionalities can be fused to the zinc finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognizes a sequence of 9 contiguous nucleotides so that two sets of zinc finger triplets are used to bind a 18 nucleotide recognition sequence. Details of ZFs can be found in Urnov et al., Nat Rev Genet (2010) 11(9):636-46. PMID: 20717154.

The DNA binding domain of a ZF is typically composed of 3-4 zinc fingers. The amino acids at positions −1, +2, +3, and +6 relative to the start of the zinc finger α-helix, which contribute to site-specific binding to the target DNA, can be changed and customized to fit specific target sequences. The other amino acids form the consensus backbone to generate ZFs with different sequence specificities. Rules for selecting target sequences for ZFs are known in the art.

The ZF disclosed herein, may be full-length or a portion thereof. In some embodiments, proteins including the ZF, or portions thereof, are referred to as “ZF variants”.

A ZF variant shares homology to ZF, or a portion thereof. For example a ZF variant is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to wild type ZF. In some embodiments, the ZF variant includes a portion of ZF, such that the portion is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the corresponding portion of wild type ZF.

Because the DNA-binding specificities of zinc finger domains can in principle be reengineered using one of various methods, customized ZFs can theoretically be constructed to target nearly any gene sequence. Publicly available methods for engineering zinc finger domains include Context-dependent Assembly (CoDA), Oligomerized Pool Engineering (OPEN), and Modular Assembly.

### Meganucleases

Meganucleases, which are commonly identified in microbes, are unique enzymes with high activity and long recognition sequences (>14 bp) resulting in site-specific digestion of target DNA. Engineered versions of naturally occurring meganucleases typically have extended DNA recognition sequences (for example, 14-40 bp).

The engineering of meganucleases is more challenging than that of ZFs and TALE because the DNA recognition and cleavage functions of meganucleases are intertwined in a single domain. Specialized methods of mutagenesis and high-throughput screening have been used to create novel meganuclease variants that recognize unique sequences and possess improved nuclease activity.

### Argonautes

The Argonaute protein family is a DNA-guided endonuclease, which has been identified in multiple species. Non-limiting examples of Argonaute proteins include Thermus thermophilus Argonaute (TtAgo), Pyrococcus furiosus Argonaute (PfAgo), Natronobacterium gregoryi Argonaute (NgAgo), homologs thereof, or modified versions thereof. Each of these unique Argonaute endonucleases have a sequence encoding a DNA guide associated with them. For example, NgAgo (SEQ ID NO: 366) is a ssDNA-guided endonuclease that binds 5′-phosphorylated ssDNA of −24 nucleotides (gDNA) in length to guide it to a target site, and then makes DNA double-strand breaks at the gDNA site (Gao et al., Nat Biotechnol (2016) 34(7):768-73. PubMed PMID: 27136078; Swarts et al., Nature 507(7491) (2014):258-61; and Swarts et al, Nucleic Acids Res. 43(10) (2015):5120-9).

In some embodiments, the DNA binding domain of any of the base-editing systems provided herein is an Argonaute domain. In some embodiments, the Argonaute domain includes an amino acid sequence that is that is at least about 60% identical, at least about 65% identical, at least about 70% identical, at least about 72.5% identical, at least about 75% identical, at least about 77.5% identical, at least about 80% identical, at least about 82.5% identical, at least about 85% identical, at least about 87.5% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, at least about 99.5% identical, or at least about 99.9% identical to SEQ ID NO: 366. In some embodiments, the Argonaute domain is SEQ ID NO: 366. In some embodiments, the Argonaute domain includes an amino acid sequence that has 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more mutations compared to any one of the amino acid sequences set forth in SEQ ID NO: 366. In some embodiments, the Argonaute domain includes an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth in SEQ ID NO: 366. In some embodiments, the present disclosure relates to nuclease inactive NgAgo (dNgAgo). Without wishing to be bound by theory, using a dNgAgo can greatly expand the bases that may be targeted.

In some embodiments, proteins including portions of Argonaute are provided. In some embodiments, proteins including Argonaute or portions thereof are referred to as “Argonaute variants.” An Argonaute variant shares homology to Argonaute, or a portion thereof. For example, an Argonaute variant is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to wild type Argonaute. In some embodiments, the Argonaute variant may have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more amino acid changes compared to wild type Argonaute. In some embodiments, the Argonaute variant includes a portion of Argonaute, such that the Argonaute portion is at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the corresponding portion of wild type Argonaute. In some embodiments, the Argonaute portion is at least 50%, at least 55%, at least 60%, at least 65%, at least 70%, at least 72.5%, at least 75%, at least 77.5%, at least 80%, at least 82.5%, at least 85%, at least 87.5%, at least 90%, at least 91%, at least 92%, at least 93%, at least 94%, at least 95%, at least 96%, at least 97%, at least 98%, at least 99%, at least 99.5%, or at least 99.9% of the amino acid length of a corresponding wild type Argonaute. In some embodiments, the Argonaute portion is at least 100 amino acids in length. In some embodiments, the Argonaute portion is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 amino acids in length. Additional suitable Argonaute domains (e.g., from other bacterial species) will be apparent to those of skill in the art based on this disclosure and knowledge in the field, and are within the scope of this disclosure.

The term “linker,” as used herein, refers to a chemical group or a molecule linking two molecules or moieties, e.g., two domains of a base-editing system, such as, for example, a nuclease inactive Cas domain (e.g., a nuclease inactive Cas9 domain or a nuclease inactive Cas12a domain) and a nucleic acid editing domain (e.g., a deaminase domain). In some embodiments, a linker joins a gRNA binding domain of an RNA-programmable nuclease, including a Cas nuclease domain (e.g., a Cas9 nuclease domain or a Cas12a nuclease domain), and the catalytic domain of a nucleic-acid editing protein. In some embodiments, a linker joins a dCas domain (e.g., a dCas9 domain or a dCas12a domain) and a nucleic-acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is in the range of 5-100 amino acids in length. In some embodiments, the linker is 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

In some embodiments, the linker includes (GGGS)ₙ(SEQ ID NO: 265), (GGGGS)ₙ (SEQ ID NO: 5), (G)ₙ, (EAAAK)ₙ(SEQ ID NO: 6), (GGS)ₙ, (SGGS)ₙ(SEQ ID NO: 1877), SGSETPGTSESATPES (SEQ ID NO: 7), or (XP)ₙ motif, or a combination of any of these, wherein n is independently an integer between 1 and 30, and wherein X is any amino acid. In some embodiments, the linker includes a (GGS)ₙ motif, wherein n is 1, 3, or 7. In some embodiments, the linker includes a (GGS)ₙ motif, wherein n is 1, 2, 3, 4, 5, 6, 7, 8, 9, 10, 11, 12, 13, 14, or 15. In some embodiments, the linker includes an amino acid sequence of SGSETPGTSESATPES (SEQ ID NO: 7). The length of the linker can influence the base to be edited, as illustrated in the Examples. For example, a linker of 3-amino-acid long (e.g., (GGS)₁) may give a 2-5, 2-4, 2-3, 3-4 base editing window relative to the PAM sequence for a Cas9 domain, while a 9-amino-acid linker (e.g., (GGS)₃ (SEQ ID NO: 596)) may give a 2-6, 2-5, 2-4, 2-3, 3-6, 3-5, 3-4, 4-6, 4-5, 5-6 base editing window relative to the PAM sequence. A 16-amino-acid linker (e.g., the XTEN linker) may give a 2-7, 2-6, 2-5, 2-4, 2-3, 3-7, 3-6, 3-5, 3-4, 4-7, 4-6, 4-5, 5-7, 5-6, 6-7 base window relative to the PAM sequence for a Cas9 domain with exceptionally strong activity, and a 21-amino-acid linker (e.g., (GGS)₇ (SEQ ID NO: 597)) may give a 3-8, 3-7, 3-6, 3-5, 3-4, 4-8, 4-7, 4-6, 4-5, 5-8, 5-7, 5-6, 6-8, 6-7, 7-8 base editing window relative to the PAM sequence. Varying linker length may allow the base-editing systems of the disclosure to edit nucleobases at different distances. It is to be understood that the linker lengths described as examples here are not meant to be limiting.

In some embodiments, the second protein includes an enzymatic domain. In some embodiments, the enzymatic domain is a nucleic acid editing domain. Such a nucleic acid editing domain may be, without limitation, a nuclease, a nickase, a recombinase, a deaminase, a methyltransferase, a methylase, an acetylase, or an acetyltransferase. Non-limiting exemplary binding domains that may be used in accordance with this disclosure include transcriptional activator domains and transcriptional repressor domains.

### Cytidine Deaminase Domains

In some aspects, the present disclosure relates to nucleic acid editing domains. In some embodiments, the nucleic acid editing domain can catalyze a C to U base change. In some embodiments, the nucleic acid editing domain is a deaminase domain. In some embodiments, the deaminase is a cytidine deaminase.

The term “deaminase” or “deaminase domain,” as used herein, refers to a protein or enzyme that catalyzes a deamination reaction. In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytidine deaminase domain, catalyzing the hydrolytic deamination of cytosine to uracil.

In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism, that does not occur in nature. For example, in some embodiments, the deaminase or deaminase domain is at least 50% identical, at least 55% identical, at least 60% identical, at least 65% identical, at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to a naturally-occurring deaminase from an organism.

One exemplary suitable type of nucleic acid editing domain is a cytidine deaminase, for example, of the APOBEC family. The apolipoprotein B mRNA-editing complex (APOBEC) family of cytidine deaminase enzymes encompasses eleven proteins that serve to initiate mutagenesis in a controlled and beneficial manner (Conticello, Genome Biol (2008) 9(6):229. PMID: 18598372). One family member, activation-induced cytidine deaminase (AID), is responsible for the maturation of antibodies by converting cytosines in ssDNA to uracils in a transcription-dependent, strand-biased fashion (Reynaud et al., Nat Immunol (2003) 4(7):631-8). The apolipoprotein B editing complex 3 (APOBEC3) enzyme provides protection to human cells against a certain HIV-1 strain via the deamination of cytosines in reverse-transcribed viral ssDNA (Bhagwat, DNA Repair (Amst) (2004) 3(1):85-9. PMID: 14697763). These proteins all require a Zn2+-coordinating motif (His-X-Glu-X₂₃₋₂₆-Pro-Cys-X₂₋₄-Cys; SEQ ID NO: 598) and bound water molecule for catalytic activity. The Glu residue acts to activate the water molecule to a zinc hydroxide for nucleophilic attack in the deamination reaction. Each family member preferentially deaminates at its own particular “hotspot”, ranging from WRC (W is A or T, R is A or G) for hAID, to TTC for hAPOBEC3F (Navaratnam et al., Int J Hematol (2006) 83(3): 195-200. PMID: 16720547). A recent crystal structure of the catalytic domain of APOBEC3G revealed a secondary structure of a five-stranded β-sheet core flanked by six α-helices, which is believed to be conserved across the entire family (Holden et al., Nature (2008) 456(7218): 121-4. PMID: 18849968). The active center loops have been shown to be responsible for both ssDNA binding and in determining “hotspot” identity (Chelico et al., J Biol Chem (2009) 284(41). 27761-5. PMID: 19684020). Overexpression of these enzymes has been linked to genomic instability and cancer, thus highlighting the importance of sequence-specific targeting (Pham et al., Biochemistry (2005) 44(8):2703-15. PMID 15723516).

In some embodiments, the deaminase is an apolipoprotein B mRNA-editing complex (APOBEC) family deaminase. In some embodiments, the deaminase is an APOBEC1 deaminase, an APOBEC2 deaminase, an APOBEC3 deaminase, or an APOBEC4 deaminase. In some embodiments, the deaminase is an APOBEC3A deaminase, an APOBEC3B deaminase, an APOBEC3C deaminase, an APOBEC3D deaminase, an APOBEC3E deaminase, an APOBEC3F deaminase, an APOBEC3G deaminase, or an APOBEC3H deaminase. In some embodiments, the deaminase is an activation-induced deaminase (AID). In some embodiments, the deaminase is a vertebrate deaminase. In some embodiments, the deaminase is an invertebrate deaminase. In some embodiments, the deaminase is a human, chimpanzee, gorilla, monkey, cow, dog, rat, or mouse deaminase. In some embodiments, the deaminase is a human deaminase. In some embodiments, the deaminase is a rat deaminase, e.g., rAPOBEC1. In some embodiments, the deaminase is a Petromyzon marinus cytidine deaminase 1 (pmCDA1). In some embodiments, the deminase is a human APOBEC3G (SEQ ID NO: 275). In some embodiments, the deaminase is a portion of the human APOBEC3G (SEQ ID NO: 1893). In some embodiments, the deaminase is a human APOBEC3G variant including a D316R_D317R mutation (SEQ ID NO: 1892). In some embodiments, the deaminase is a portion of the human APOBEC3G and including mutations corresponding to the D316R_D317R mutations in SEQ ID NO: 275 (SEQ ID NO: 1894).

In some embodiments, the nucleic acid editing domain is an APOBEC family deaminase domain and is at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to the deaminase domain of any one of SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894. In some embodiments, the nucleic acid editing domain includes the amino acid sequence of any one of SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894.

Some aspects of the disclosure are based on the recognition that modulating the deaminase domain catalytic activity of any of the base-editing systems provided herein, for example by making point mutations in the deaminase domain, affect the processivity of the base-editing systems. For example, mutations that reduce, but do not eliminate, the catalytic activity of a deaminase domain within a base-editing system can make it less likely that the deaminase domain will catalyze the deamination of a residue adjacent to a target residue, thereby narrowing the deamination window. The ability to narrow the deamination window may prevent unwanted deamination of residues adjacent of specific target residues, which may decrease or prevent off-target effects.

In some embodiments, any of the base-editing systems provided herein include a deaminase domain (e.g., a cytidine deaminase domain) that has reduced catalytic deaminase activity. In some embodiments, any of the base-editing systems provided herein include a deaminase domain (e.g., a cytidine deaminase domain) that has a reduced catalytic deaminase activity as compared to an appropriate control. For example, the appropriate control may be the deaminase activity of the deaminase prior to introducing one or more mutations into the deaminase. In other embodiments, the appropriate control may be a wild-type deaminase. In some embodiments, the appropriate control is a wild-type apolipoprotein B mRNA-editing complex (APOBEC) family deaminase. In some embodiments, the appropriate control is an APOBEC 1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, or an APOBEC3H deaminase. In some embodiments, the appropriate control is an activation induced deaminase (AID). In some embodiments, the appropriate control is a cytidine deaminase 1 from Petromyzon marinus (pmCDA1). In some embodiments, the deaminase domain may be a deaminase domain that has at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% less catalytic deaminase activity as compared to an appropriate control.

In some embodiments, any of the base-editing systems provided herein include an APOBEC deaminase including one or more mutations selected from the group of H121X, H122X, R126X, R126X, R118X, W90X, W90X, or R132X of rAPOBEC1 (SEQ ID NO: 284), or one or more corresponding mutations in another APOBEC deaminase, wherein X is any amino acid. In some embodiments, any of the base-editing systems provided herein include an APOBEC deaminase including one or more mutations selected from the group of H121R, H122R, R126A, R126E, R118A, W90A, W90Y, or R132E of rAPOBEC1 (SEQ ID NO: 284), or one or more corresponding mutations in another APOBEC deaminase.

In some embodiments, any of the base-editing systems provided herein include an APOBEC deaminase including one or more mutations selected from the group of D316X, D317X, R320X, R320X, R313X, W285X, W285X, or R326X of hAPOBEC3G (SEQ ID NO: 275), or one or more corresponding mutations in another APOBEC deaminase, wherein X is any amino acid. In some embodiments, any of the base-editing systems provided herein include an APOBEC deaminase including one or more mutations selected from the group of D316R, D317R, R320A, R320E, R313A, W285A, W285Y, or R326E of hAPOBEC3G (SEQ ID NO: 275), or one or more corresponding mutations in another APOBEC deaminase.

In some embodiments, any of the base-editing systems provided herein include an APOBEC deaminase including a H121R and a H122R mutation of rAPOBEC1 (SEQ ID NO: 284), or one or more corresponding mutations in another APOBEC deaminase. In some embodiments, any of the base-editing systems provided herein include an APOBEC deaminase including a R126A mutation of rAPOBEC1 (SEQ ID NO: 284), or one or more corresponding mutations in another APOBEC deaminase. In some embodiments, any of the base-editing systems provided herein include an APOBEC deaminase including a R126E mutation of rAPOBEC1 (SEQ ID NO: 284), or one or more corresponding mutations in another APOBEC deaminase. In some embodiments, any of the base-editing systems provided herein include an APOBEC deaminase including a R118A mutation of rAPOBEC1 (SEQ ID NO: 284), or one or more corresponding mutations in another APOBEC deaminase. In some embodiments, any of the base-editing systems provided herein include an APOBEC deaminase including a W90A mutation of rAPOBEC1 (SEQ ID NO: 284), or one or more corresponding mutations in another APOBEC deaminase. In some embodiments, any of the base-editing systems provided herein include an APOBEC deaminase including a W90Y mutation of rAPOBEC1 (SEQ ID NO: 284), or one or more corresponding mutations in another APOBEC deaminase. In some embodiments, any of the base-editing systems provided herein include an APOBEC deaminase including a R132E mutation of rAPOBEC1 (SEQ ID NO: 284), or one or more corresponding mutations in another APOBEC deaminase. In some embodiments, any of the base-editing systems provided herein include an APOBEC deaminase including a W90Y and a R126E mutation of rAPOBEC1 (SEQ ID NO: 284), or one or more corresponding mutations in another APOBEC deaminase. In some embodiments, any of the base-editing systems provided herein include an APOBEC deaminase including a R126E and a R132E mutation of rAPOBEC1 (SEQ ID NO: 284), or one or more corresponding mutations in another APOBEC deaminase. In some embodiments, any of the base-editing systems provided herein include an APOBEC deaminase including a W90Y and a R132E mutation of rAPOBEC1 (SEQ ID NO: 284), or one or more corresponding mutations in another APOBEC deaminase. In some embodiments, any of the base-editing systems provided herein include an APOBEC deaminase including a W90Y, R126E, and R132E mutation of rAPOBEC1 (SEQ ID NO: 284), or one or more corresponding mutations in another APOBEC deaminase.

In some embodiments, any of the base-editing systems provided herein include an APOBEC deaminase including a D316R and a D317R mutation of hAPOBEC3G (SEQ ID NO: 275), or one or more corresponding mutations in another APOBEC deaminase. In some embodiments, any of the base-editing systems provided herein include an APOBEC deaminase including a R320A mutation of hAPOBEC3G (SEQ ID NO: 275), or one or more corresponding mutations in another APOBEC deaminase. In some embodiments, any of the base-editing systems provided herein include an APOBEC deaminase including a R320E mutation of hAPOBEC3G (SEQ ID NO: 275), or one or more corresponding mutations in another APOBEC deaminase. In some embodiments, any of the base-editing systems provided herein include an APOBEC deaminase including a R313A mutation of hAPOBEC3G (SEQ ID NO: 275), or one or more corresponding mutations in another APOBEC deaminase. In some embodiments, any of the base-editing systems provided herein include an APOBEC deaminase including a W285A mutation of hAPOBEC3G (SEQ ID NO: 275), or one or more corresponding mutations in another APOBEC deaminase. In some embodiments, any of the base-editing systems provided herein include an APOBEC deaminase including a W285Y mutation of hAPOBEC3G (SEQ ID NO: 275), or one or more corresponding mutations in another APOBEC deaminase. In some embodiments, any of the base-editing systems provided herein include an APOBEC deaminase including a R326E mutation of hAPOBEC3G (SEQ ID NO: 275), or one or more corresponding mutations in another APOBEC deaminase. In some embodiments, any of the base-editing systems provided herein include an APOBEC deaminase including a W285Y and a R320E mutation of hAPOBEC3G (SEQ ID NO: 275), or one or more corresponding mutations in another APOBEC deaminase. In some embodiments, any of the base-editing systems provided herein include an APOBEC deaminase including a R320E and a R326E mutation of hAPOBEC3G (SEQ ID NO: 275), or one or more corresponding mutations in another APOBEC deaminase. In some embodiments, any of the base-editing systems provided herein include an APOBEC deaminase including a W285Y and a R326E mutation of hAPOBEC3G (SEQ ID NO: 275), or one or more corresponding mutations in another APOBEC deaminase. In some embodiments, any of the base-editing systems provided herein include an APOBEC deaminase including a W285Y, R320E, and R326E mutation of hAPOBEC3G (SEQ ID NO: 275), or one or more corresponding mutations in another APOBEC deaminase.

Some aspects of this disclosure relate to the recognition that the activity of cytidine deaminase enzymes such as APOBEC enzymes can be directed to a specific site in genomic DNA. Without wishing to be bound by any particular theory, advantages of using Cas9 as a recognition agent include (1) the sequence specificity of Cas9 can be easily altered by simply changing the sgRNA sequence; and (2) Cas9 binds to its target sequence by denaturing the dsDNA, resulting in a stretch of DNA that is single-stranded and therefore a viable substrate for the deaminase. Further, without wishing to be bound by any particular theory, advantages of using Cas12a as a recognition agent over Cas9 include (1) Cas12a variants have been described that operate at lower temperatures than available Cas9 variants, and are therefore potentially more useful in plant systems than Cas9 variants; and (2) Cas12a has a different editing window (bases available for modification) than Cas9 targeting the same DNA sequence with the same deaminase (e.g., due to differences in the position and orientation of the deaminase domain and the accessibility of the R-loop substrate). Further still, without wishing to be bound by any particular theory, advantages of using TALEs and ZFs include (1) the lack of any PAM preference, which enables editing at any base, and increased efficiency in regions of dense chromatin; and (2) TALEs and ZFs do not melt the DNA, and therefore must access targets in dsDNA, which changes the dynamics and likely sequence preferences of deamination. It should be understood that other catalytic domains, or catalytic domains from other deaminases, can also be used to generate base-editing systems with DNA binding domains (e.g., a Cas9 domain, a Cas12a domain, a Cas12h domain, a Cas12i domain, a CasX domain, a CasY domain, a C2c1 domain, a C2c2 domain, a C2c3 domain, a TALE domain, or a ZF domain), and that the disclosure is not limited in this regard.

In some embodiments, the deaminase domain and the DNA binding domain (e.g., a Cas9 domain, a Cas12a domain, a Cas12h domain, a Cas12i domain, a CasX domain, a CasY domain, a C2c1 domain, a C2c2 domain, a C2c3 domain, a TALE domain, or a ZF domain) are fused to each other via a linker. Various linker lengths and flexibilities between the deaminase domain (e.g., AID) and the DNA binding domain (e.g., a Cas9 domain, a Cas12a domain, a Cas12h domain, a Cas12i domain, a CasX domain, a CasY domain, a C2c1 domain, a C2c2 domain, a C2c3 domain, a TALE domain, or a ZF domain) can be employed (e.g., ranging from very flexible linkers of the form (GGGGS)ₙ(SEQ ID NO: 5), (GGS)ₙ, and (G)ₙ to more rigid linkers of the form (EAAAK)ₙ(SEQ ID NO: 6), (SGGS)ₙ(SEQ ID NO: 1877), SGSETPGTSESATPES (SEQ ID NO: 7) (see, e.g., Guilinger et al., Nat Biotechnol (2014) 32(6): 577-82) and (XP)ₙ)³⁶ in order to achieve the optimal length for deaminase activity for the specific application. In some embodiments, the linker includes a (GGS)ₙ motif, wherein n is 1, 3, or 7. In some embodiments, the linker includes a SGSETPGTSESATPES (SEQ ID NO: 7) motif.

Some exemplary suitable nucleic-acid editing domains, e.g., deaminases and deaminase domains, that can be fused to DNA binding domains (e.g., a Cas9 domain, a Cas12a domain, a Cas12h domain, a Cas12i domain, a CasX domain, a CasY domain, a C2c1 domain, a C2c2 domain, a C2c3 domain, a TALE domain, or a ZF domain) according to aspects of this disclosure are Human AID (SEQ ID NO: 266), Mouse AID (SEQ ID NO: 267), Dog AID (SEQ ID NO: 268), Bovine AID (SEQ ID NO: 269), Rat AID (SEQ ID NO: 1879), Mouse APOBEC-3 (SEQ ID NO: 270), Rat APOBEC-3 (SEQ ID NO: 271), Rhesus macaque APOBEC-3 G (SEQ ID NO: 272), Chimpanzee APOBEC-3 G (SEQ ID NO: 273), Green monkey APOBEC-3G (SEQ ID NO: 274), Human APOBEC-3G (SEQ ID NO: 275), Human APOBEC-3F (SEQ ID NO: 276), Human APOBEC-3B (SEQ ID NO: 277), Rat APOBEC-3B (SEQ ID NO: 1883), Bovine APOBEC-3B (SEQ ID NO: 1884), Chimpanzee APOBEC-3B (SEQ ID NO: 1885), Human APOBEC-3C (SEQ ID NO: 278), Gorilla APOBEC3C (SEQ ID NO: 1880), Human APOBEC-3A (SEQ ID NO: 279), Rhesus macaque APOBEC-3A (SEQ ID NO: 1881), Bovine APOBEC-3A (SEQ ID NO: 1882), Human APOBEC-3H (SEQ ID NO: 280), Rhesus macaque APOBEC-3H (SEQ ID NO: 1886), Human APOBEC-3D (SEQ ID NO: 281), Human APOBEC-1 (SEQ ID NO: 282), Mouse APOBEC-1 (SEQ ID NO: 283), Rat APOBEC-1 (SEQ ID NO: 284), Human APOBEC-2 (SEQ ID NO: 1887), Mouse APOBEC-2 (SEQ ID NO: 1888), Rat APOBEC-2: (SEQ ID NO: 1889), Bovine APOBEC-2 (SEQ ID NO: 1890), Petromyzon marinus CDA1 (pmCDA1) (SEQ ID NO: 1891), Human APOBEC3G D316R_D317R (SEQ ID NO: 1892), Human APOBEC3G chain A (SEQ ID NO: 1893), Human APOBEC3G chain A D120R_D121R (SEQ ID NO: 1894). It should be understood that, in some embodiments, the active domain of the respective sequence can be used, e.g., the domain without a localizing signal (nuclear localization sequence, without nuclear export signal, cytoplasmic localizing signal).

In some embodiments, base-editing systems as provided herein include the full-length amino acid of a nucleic acid editing enzyme, e.g., one of the sequences provided above. In other embodiments, however, base-editing systems as provided herein do not include a full-length sequence of a nucleic acid editing enzyme, but only a portion thereof. For example, in some embodiments, a base-editing system provided herein includes a DNA binding domain (e.g., a Cas9 domain, a Cas12a domain, a Cas12h domain, a Cas12i domain, a CasX domain, a CasY domain, a C2c1 domain, a C2c2 domain, a C2c3 domain, an Argonaute domain, a TALE domain, or a ZF domain) and a portion of a nucleic acid editing enzyme. In some embodiments, the portion of the nucleic acid editing enzyme includes a nucleic acid editing motif. In some embodiments, the nucleic acid editing motif is at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% to SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, or SEQ ID NO: 349. In some embodiments, the nucleic acid editing motif is SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, or SEQ ID NO: 349. Additional suitable sequences of such motifs will be apparent to those of skill in the art.

Additional suitable nucleic-acid editing enzyme sequences, e.g., deaminase enzyme and domain sequences, that can be used according to aspects of this invention, e.g., that can be fused to a nuclease inactive DNA binding domain (e.g., a nuclease inactive Cas9 (dCas9), a nuclease inactive Cas12a (dCas12a), a TALE, or a ZF), will be apparent to those of skill in the art based on this disclosure. In some embodiments, such additional enzyme sequences include deaminase enzyme or deaminase domain sequences that are at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, or at least 99% identical to the sequences provided herein.

Additional suitable strategies for generating base-editing systems including a DNA binding domain and a deaminase domain will be apparent to those of skill in the art based on this disclosure in combination with the general knowledge in the art. Suitable strategies for generating base-editing systems according to aspects of this disclosure using linkers or without the use of linkers will also be apparent to those of skill in the art in view of the instant disclosure and the knowledge in the art. For example, Gilbert et al., Cell (2013) 154(2):442-51, showed that C-terminal fusions of Cas9 with VP64 using 2 NLS's as a linker (SPKKKRKVEAS, SEQ ID NO: 599), can be employed for transcriptional activation. Mali et al, CAS9 transcriptional activators for target specificity screening and paired nickases for cooperative genome engineering. Nat Biotechnol. 2013; 31(9):833-8, reported that C-terminal fusions with VP64 without linker can be employed for transcriptional activation. And Maeder et al., Nat Methods (2013) 10: 977-979, reported that C-terminal fusions with VP64 using a Gly4Ser (SEQ ID NO: 5) linker can be used as transcriptional activators. Recently, dCas9-FokI nuclease fusions have successfully been generated and exhibit improved enzymatic specificity as compared to the parental Cas9 enzyme (In Guilinger et al., Nat. Biotechnol (2014) 32(6): 577-82, and in Tsai et al., Nat Biotechnol (2014) 32(6):569-76. PMID: 24770325, a SGSETPGTSESATPES (SEQ ID NO: 7) or a GGGGS (SEQ ID NO: 5) linker was used in FokI-dCas9 fusion proteins, respectively).

### Non-Protein Uracil-DNA Glycosylase Inhibitors

In some aspects, the present disclosure relates to non-protein uracil-DNA glycosylase inhibitors (npUGI), also known as uracil glycosylase inhibitors. In some embodiments, the npUGI is selected from the group including an organochemical inhibitor of uracil glycosylase, a nucleic acid based inhibitor of uracil-DNA glycosylase, or a RNA guide of a dCas nuclease (e.g., dCas9, dCas12a, dCas12h, dCas12i, dCasX, dCasY, dC2c1, dC2c2, or dC2c3) with a poly dU (deoxy-Uracil) extension. It is to be understood that “non-protein” means without one or more amino acid, polypeptide, or protein components (i.e., without any amino acid, polypeptide, or protein components).

Without wishing to be bound by any particular theory, cellular DNA-repair response to the presence of U:G heteroduplex DNA may decrease nucleobase editing efficiency in cells. For example, uracil-DNA glycosylase (UDG) catalyzes removal of U from DNA in cells, which may initiate base excision repair, with reversion of the U:G pair to a C:G pair as the most common outcome. As demonstrated in the Examples below, non-protein uracil-DNA glycosylase inhibitors (npUGI) inhibit UDG activity (e.g., completely inhibit UDG activity). It should be understood that the use of a npUGI increases the editing efficiency of a nucleic acid editing domain that is capable of catalyzing a C to U change. For example, systems including a npUGI may be more efficient in deaminating C residues.

In some embodiments, the npUGI is an organochemical inhibitor of UDG. For example, an organochemical inhibitor of UDG may be a small molecule inhibitor of UDG.

In some embodiments, the npUGI is a nucleic acid based inhibitor of UDG. For example, a RNA molecule, an antisense oligonucleotide molecule, a microRNA (miRNA) molecule, a short hairpin RNA (shRNA) molecule, a double stranded RNA (dsRNA) molecule, a small interfering RNA (siRNA) molecule. In some embodiments, the npUGI is a DNA molecule capable of inhibiting a uracil-DNA glycosylase base-excision repair enzyme. In some embodiments, the npUGI is a RNA-DNA chimera capable of inhibiting a uracil-DNA glycosylase base-excision repair enzyme.

In some embodiments, the npUGI is a RNA guide of a dCas nuclease with a poly dU (deoxy-Uracil) extension. For example, this may be a poly deoxy-uracil (dU) extension added to guide RNA capable of inhibiting an UDG.

In some embodiments, the npUGI inhibits UDG in plants. In some embodiments, the npUGI inhibits the plant UDGs of Table 1. In some embodiments, the npUGI inhibits animal UDGs.

### Small Molecule Inhibitors of Uracil-DNA Glycosylase

In some aspects, the present disclosure relates to base-editing systems including small molecule inhibitors of UDG. In some embodiments, the small molecule inhibitors include a compound of formula (I) or a pharmaceutically acceptable salt thereof:

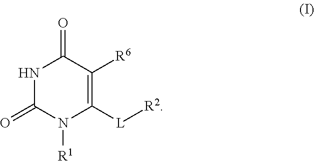

R¹ is H, a furanose carbohydrate, or a derivative thereof, a pyranose carbohydrate, or a derivative thereof, a carbohydrate mimetic, C₁₋₁₆alkyl, C₁₋₁₆alkenyl, C₁₋₁₆alkynyl, C₁₋₁₆alkoxy, or C₆₋₂₀aryl, wherein the furanose carbohydrate, or a derivative thereof, pyranose carbohydrate, or a derivative thereof, carbohydrate mimetic, C₁₋₁₂alkyl, C₁₋₁₂alkenyl, C₁₋₁₂alkynyl, C₁₋₁₂alkoxy, or C₆₋₂₀aryl is independently optionally substituted with one or more substituents selected from the group of hydroxyl, halo, cyano, NO₂, N(R⁴)(R⁵), C₁₋₁₂alkoxy, or C₆₋₂₀aryl, wherein the C₆₋₂₀aryl is further optionally substituted with one or more substituents selected from the group of C₁₋₁₂alkyl, C₁₋₁₂alkenyl, C₁₋₁₂alkynyl, C₁₋₁₂alkoxy, hydroxyl, halo, cyano, NO₂, or N(R⁴)(R⁵).

L is O, S, or NR³, wherein R³ is H or C₁₋₁₆alkyl.

R² is H, N(R⁴)(R⁵), or C₆₋₂₀aryl, wherein the C₆₋₂₀aryl is independently substituted with one or more substituents selected from the group of C₁₋₁₆alkyl, C₁₋₁₆alkenyl, C₁₋₁₆alkynyl, or C₁₋₁₆alkoxy.

R⁴ and R⁵ are each independently H or C₆₋₂₀aryl.

R⁶ is H or halo.

In some embodiments of the compound of formula (I), or a pharmaceutically acceptable salt thereof, R¹ is H.

In some embodiments of the compound of formula (I), or a pharmaceutically acceptable salt thereof, R¹ is C₁₋₁₆alkyl, wherein the C₁₋₁₆alkyl is optionally substituted with one or more substituents selected from the group of hydroxyl, halo, cyano, NO₂, N(R⁴)(R⁵), C₁₋₁₆alkoxy, or C₆₋₂₀aryl, wherein the C₆₋₂₀aryl is further optionally substituted with one or more substituents selected from the group of C₁₋₁₆alkyl, C₁₋₁₆alkenyl, C₁₋₁₆alkynyl, C₁₋₁₆alkoxy, hydroxyl, halo, cyano, NO₂, or N(R⁴)(R⁵), wherein R₄ and R₅ are each independently H or C₆₋₂₀aryl. In some embodiments of the compound of formula (I), or a pharmaceutically acceptable salt thereof, L is *—N(R³)—**, wherein ** indicates the point of attachment to the R² moiety and * indicates the point of attachment to the remainder of the molecule. In certain variations, R³ is H. In other variations, R³ is C₁₋₁₆alkyl. In other embodiments, L is —O—.

In some embodiments of the compound of formula (I), or a pharmaceutically acceptable salt thereof, R² is C₆₋₂₀aryl, wherein the C₆₋₂₀aryl is independently substituted with one or more substituents selected from the group of C₁₋₁₆alkyl, C₁₋₁₆alkenyl, C₁₋₁₆alkynyl, or C₁₋₁₆alkoxy.

In some embodiments of the compound of formula (I), or a pharmaceutically acceptable salt thereof, R⁶ is H. In other embodiments, R⁶ is halo. In some embodiments, R⁶ is fluorine. In some embodiments, R⁶ is chlorine. In other embodiments, R⁶ is bromine. In still other embodiments, R⁶ is iodine.

In some embodiments, the compound of formula (I) is, for example:

1-methoxyethyl-6-(p-n-octylanilino)uracil
6-(phenylhydrazino)uracil
6-(4-octylphenoxy)uracil
1-methyl-6-(4-hexylanilino)uracil
1-ethyl-6-(4-hexylanilino)uracil
1-propyl-6-(4-hexylanilino)uracil
1-(2-hydroxyethyl)-6-(4-octylanilino)uracil
1-(2-methoxyethyl)-6-(4-hexylanilino)uracil

or a pharmaceutically acceptable salt thereof.

In some embodiments, the base-editing system including a small molecule inhibitor of any of the above embodiments further includes a pharmaceutically acceptable carrier, diluent, or excipient.

In some embodiments, the base-editing system including a small molecule inhibitor including the compound of formula (I) is incorporated into a macromolecule. In certain embodiments, the macromolecule is an oligonucleotide.

Additional details regarding small molecule inhibitors of UDG may be found in Suksangpleng et al., Malar J (2014) 17; 13:149. PMID: 24742318; Pregnolato et al., Nucleosides Nucleotides (1999) 18(4-5):709-11. PMID: 10432670; Sun et al., J Med Chem (1999) 42(13):2344-50. PMID: 10395474; Focher et al., Biochem J. (1993) 15; 292 (Pt 3):883-9. PMID: 8391260; Argnani et al., Virology (1995) 211(1):307-11. PMID: 7645226; Hendricks et al., J Chem Inf Model (2014) 54(12):3362-72. PMID: 25369428; Huang et al., J Am Chem Soc (2009) 131(4): 1344-1345. PMID: 19173657. Krosky et al., Nucleic Acids Res (2006) 34(20):5872-9. PMID: 17062624; Jiang et al., Bioorg Med Chem (2006) 14(16):5666-72. PMID: 16678429; Chung et al., Nat Chemm Biol (2009) 5(6):407-13. PMID: 19396178; Braunheim et al., Methods Enzymol (1999) 308: 398-426. PMID: 10507012; Jiang et al., Bioorg Chem (2004) 32(4):244-62. PMID: 15210339; Purmal et al., Biochemistry (1996) 35(51):16630-7. PMID: 8987998; Rosler et al., Nucleosides Nucleotides Nucelic Acids (2000) 19(10-12):1505-16. PMID: 11200255; Mikalkenas et al., J Enzyme Inhib Med Chem (2018) 33(1): 384-389. PMID: 29372656; Ono et al., Nucleic Acids Symp Ser (2000) (44):127-8. PMID: 12903301; and Kubareva et al., Gene (1995) 157(1-2):167-71. PMID: 7607485.

“Furanose carbohydrate” refers to a carbohydrate that includes a five-membered ring system containing four carbon atoms and one oxygen atom. The ring is saturated in that it contains no carbon-carbon double bonds.

“Pyranose carbohydrate” refers to a carbohydrate that includes a six-membered ring system containing five carbon atoms and one oxygen atom. The ring is saturated in that it contains no carbon-carbon double bonds.

In some embodiments, examples of furanose carbohydrates, pyranose carbohydrates, and derivatives and mimetics thereof, include, but are not limited to:

2-((2R,3S,4R,5R)-3,4-dihydroxy-5-
(hydroxymethyl)tetrahydrofuran-2-
yl)thiazole-4-carboxamide
1-((2R,4R,5S)-4-azido-5-
(hydroxymethyl)tetrahydrofuran-2-
yl)-5-methylpyrimidine-2,4(1H,3H)-
dione
(2R,3S,4R,5R)-2-(6-amino-9H-purin-9-
yl)-5-
(hydroxymethyl)tetrahydrofuran-3,4-
diol
(1S,2R,3R,5R)-3-(2-amino-6-
(cyclopropylamino)-9H-purin-9-yl)-5-
(hydroxymethyl)cyclopentane-1,2-diol
(2S,3S,4R,5R)-2-(7-amino-1H-
pyrazolo[4,3-d]pyrimidin-3-yl)-5-
(hydroxymethyl)tetrahydrofuran-3,4-
diol
3-((2S,3S,4R,5R)-3,4-dihydroxy-5-
(hydroxymethyl)tetrahydrofuran-2-
yl)-1H-pyrrole-2,5-dione
1-((2R,3S,4R,5R)-3,4-dihydroxy-5-
(hydroxymethyl)tetrahydrofuran-2-
yl)-5-fluoropyrimidine-2,4(1H,3H)-
dione
2-((1R,4S,5R)-4,5-dihydroxy-3-
(hydroxymethyl)cyclopent-2-en-1-yl)-
3H-1,2l4,4-triazole-5-carboxamide
(1R,2S,5R)-5-(6-amino-9H-purin-9-yl)-
3-(hydroxymethyl)cyclopent-3-ene-
1,2-diol
4-amino-1-((2R,5S)-2-
(hydroxymethyl)-1,3-oxathiolan-5-
yl)pyrimidin-2(1H)-one
(R)-4-amino-1-(4-hydroxybuta-1,2-
dien-1-yl)pyrimidin-2(1H)-one
((2S,3R,4R)-4-(6-amino-9H-purin-9-
yl)oxetane-2,3-diyl)dimethanol
3-((2R,3R,3aS,4aS,5S,6S,7S,8aR,9aR)-
2-(6-amino-9H-purin-9-yl)-4a,5,6-
trihydroxy-3-
methoxydecahydrofuro[3,2-
b]pyrano[2,3-e]pyran-7-yl)-3l5-
pentan-3-one
2-amino-9-((2-
hydroxyethoxy)methyl)-7l2,9l4-purin-
6(1H)-one
(E)-N-((2S,3R,4R,5R)-2-
(((2R,3R,4R,5S,6R)-3-acetamido-4,5-
dihydroxy-6-
(hydroxymethyl)tetrahydro-2H-pyran-
2-yl)oxy)-6-((R)-2-((2R,3S,4R,5R)-5-
(2,4-dioxo-3,4-dihydropyrimidin-
1(2H)-yl)-3,4-
dihydroxytetrahydrofuran-2-yl)-2-
hydroxyethyl)-4,5-
dihydroxytetrahydro-2H-pyran-3-yl)-
13-methyltetradec-2-enamide

“Alkyl” refers to a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁₋₂₀alkyl”), having 1 to 16 carbon atoms (a “C₁₋₆alkyl”), having 1 to 10 carbon atoms (a “C₁₋₁₀alkyl”), having 6 to 10 carbon atoms (a “C₆₋₁₀alkyl”), having 1 to 6 carbon atoms (a “C₁₋₆alkyl”), having 2 to 6 carbon atoms (a “C₂₋₆alkyl”), or having 1 to 4 carbon atoms (a “C₁₋₄alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkenyl” refers to a straight or branched chain alkyl or substituted alkyl group as defined elsewhere herein having at least one carbon-carbon double bond. Alkenyl groups may be optionally substituted.

“Alkynyl” refers to and includes a straight or branched chain alkyl or substituted alkyl group as defined elsewhere herein having at least one carbon-carbon triple bond. Alkynyl groups may be optionally substituted.

“Alkoxy” refers to the group R—O—, where R is alkyl; and includes, by way of example, methoxy, ethoxy, n-propoxy, iso-propoxy, n-butoxy, tert-butoxy, sec-butoxy, n-pentoxy, n-hexyloxy, 1,2-dimethylbutoxy, and the like.

“Aryl” or “Ar” refers to an unsaturated aromatic carbocyclic group having a single ring (e.g., phenyl) or multiple condensed rings (e.g., naphthyl or anthryl), in which the condensed rings may or may not be aromatic. Particular aryl groups are those having from 6 to 14 annular carbon atoms (a “C₆-C₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Cyano” refers to a —C≡N group.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, diluent, stabilizer, or preservative.

“Oligonucleotide” refers to polynucleotides of between about 5 and about 100 nucleotides of single- or double-stranded DNA. Oligonucleotides are also known as oligomers or oligos and may be isolated from genes, or chemically synthesized by methods known in the art. A “primer” refers to an oligonucleotide, usually single-stranded, that provides a 3′-hydroxyl end for the initiation of enzyme-mediated nucleic acid synthesis. The primer sequence need not reflect the exact sequence of the template. “PCR primers” refer to primers used in “polymerase chain reaction” or “PCR,” a method for amplifying a DNA base sequence using a heat-stable polymerase such as Taq polymerase, and two oligonucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce exponential and highly specific amplification of the desired sequence. PCR also can be used to detect the existence of the defined sequence in a DNA sample.

### Nucleic Acid Inhibitors of Uracil-DNA Glycosylase

In some embodiments, the present disclosure relates to nucleic acid based inhibitors of uracil-DNA glycosylase. In some embodiments, the nucleic acid based inhibitor of uracil-DNA glycosylase may be a RNA molecule, an antisense oligonucleotide, a DNA molecule, or a RNA/DNA chimera molecule. In some embodiments, the RNA molecule is a double-stranded RNA (dsRNA) molecule, a small interfering RNA (siRNA) molecule, a microRNA (miRNA) molecule, or a short hairpin RNA (shRNA) molecule. In some embodiments, the nucleic acid uracil-DNA glycosylase inhibitory molecule (e.g., a dsRNA molecule, a siRNA molecule, a miRNA molecule, a shRNA molecule, a DNA molecule, or a RNA/DNA chimera molecule) knocks down the non-exhaustive list of uracil-DNA glycosylase targets identified in Table 1. In some embodiments, additional target sequences can be accessed in standard genome databases (e.g., NCBI, Pfam, SCOP, Interior) for most species of interest. In some embodiments, the RNA molecule includes a nucleic acid sequence that is at least 60% identical, at least 65% identical, at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to any one of the nucleic acid sequences of SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments, the RNA molecule includes any one of the nucleic acids of SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments, the RNA molecule includes a nucleic acid sequence that has 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more mutations compared to any one of the nucleic acid sequences set forth in SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments, the RNA molecule includes a nucleic acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous nucleic acid residues as compared to any one of the nucleic acid sequences set forth in SEQ ID NO: 373, SEQ ID NO: 374, SEQ ID NO: 375, SEQ ID NO: 376, SEQ ID NO: 377, SEQ ID NO: 378, or SEQ ID NO: 379.

In some embodiments, the DNA molecule includes a nucleic acid sequence that is at least 60% identical, at least 65% identical, at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to any one of the nucleic acid sequences set forth in SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments, the DNA molecule includes any one of the nucleic acids of SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments, the DNA molecule includes a nucleic acid sequence that has 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more mutations compared to any one of the nucleic acid sequences set forth in SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments, the DNA molecule includes a nucleic acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous nucleic acid residues as compared to any one of the nucleic acid sequences set forth in SEQ ID NO: 373, SEQ ID NO: 374, SEQ ID NO: 375, SEQ ID NO: 376, SEQ ID NO: 377, SEQ ID NO: 378, or SEQ ID NO: 379.

In some embodiments, the RNA/DNA chimera molecule includes a nucleic acid sequence that is at least 60% identical, at least 65% identical, at least 70% identical, at least 72.5% identical, at least 75% identical, at least 77.5% identical, at least 80% identical, at least 82.5% identical, at least 85% identical, at least 87.5% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, or at least 99.9% identical to any one of the nucleic acid sequences set forth in SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments, the RNA/DNA chimera molecule includes any one of the nucleic acids of SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments, the RNA/DNA chimera molecule includes a nucleic acid sequence that has 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more mutations compared to any one of the nucleic acid sequences set forth in SEQ ID NO: 373, SEQ ID NO: 374, SEQ ID NO: 375, SEQ ID NO: 376, SEQ ID NO: 377, SEQ ID NO: 378, or SEQ ID NO: 379. In some embodiments, the RNA/DNA chimera molecule includes a nucleic acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous nucleic acid residues as compared to any one of the nucleic acid sequences set forth in SEQ ID NO: 373, SEQ ID NO: 374, SEQ ID NO: 375, SEQ ID NO: 376, SEQ ID NO: 377, SEQ ID NO: 378, or SEQ ID NO: 379.

Additional nucleic acid based inhibitors of UDG will be apparent to those of skill in the art based on this disclosure and knowledge in the field, and are within the scope of this disclosure. Details of nucleic acid based inhibitors may be found in Roldan-Arjone et al., Front Plant Sci. (2019) 10: 1055. PMID: 31543887; and Dalakouras et al., Front Plant Sci. (2016) 7:1327. PMID: 27625678.

TABLE 1
Exemplary Uracil-DNA Glycosylase (UDG) Targets in Plants

| Species | RefSeq No.: | Amino Acid Sequence |
|---|---|---|
| Sorghum bicolor | XP_021318175.1 | MAPSPPTPAAPKTIADFFARPAKRLRAGSAAPAASLSSSPSSLSPE |
| | | QRRRADTNLALARARRNLRLAESKAQASGGAPKLEELLVEETW |
| | | AEALHGELRKPYALELCRFVAHERLHGPLPVYPPPHLVFHALNA |
| | | TPFDRVKAVIIGQDPYHGPGQAMGLSFSVPEGIKIPSSLGNIFKEL |
| | | EKDLGCTVPSHGNLERWAVQGVLMLNTVLTVREHQANSHAKK |
| | | GWEQFTDAVIKTVSQKKSGLVFLLWGNSAQSKTRLIDETKHHIL |
| | | KSAHPSGLSANRGFFGCRHFSKTNQILEKLGLSAIDWQL (SEQ ID |
| | | NO: 373) |
| Zea mays | NP_001150773.2 | MPPSPPTPAAPKTIADFFARPAKRLRAAPAASLSSNSSPSSLSPEQ |
| | | RRRADTNLALARARRNLRLAESKAQASGGAPKLEELLVEETWA |
| | | EALRGELRKSYALELCRFVAHERLHGPLPVYPPPHLVFHALNAT |
| | | PFDRVKAVIIGQDPYHGPGQAMGLSFSVPEGIKIPSSLGNIFKELK |
| | | KDLGCTVPSHGNLERWAVQGVLMLNTVLTVREHQANSHAKKG |
| | | WEQFTDEVIKTISQKKSGLVFLLWGNSAQSKTRLIDETKHHILKS |
| | | AHPSGLSANRGFFGCRHFSKTNEILEKLGLSAIDWQL (SEQ ID |
| | | NO: 374) |
| Solanum | XP_010315595.1 | MASSSSKTLKDLWKQPAAKRLKQVSSTENFISSALASSSSRKDC |
| lycopersicum | | DEDPKDVVSSTPEQNSRMEFNRSLAKSKRNLKLCSDKISKLNAN |
| | | GEGGGYVKLQELLIEETWLEALPGEFEKTYAGNLCKFVEKEISG |
| | | GVPIYPPLHLIFNALNTTAFDRIKAVIIGQDPYHGPGQAMGLSFS |
| | | VPKGVKVPSSLLNIYKELKQDLGCSIPLHGNLEQWAVQGVLLLN |
| | | AVLTVRHHQANSHANKGWEQFTDAIIKTISKKKEGVVFILWGN |
| | | YAQAKARLVDETKHHILKSAHPSGLSANRGFFGCRAWFPLTSQ |
| | | VLCSPTCQRNKWKQL (SEQ ID NO: 375) |
| Oryza saliva | XP_015634353.1 | MAPPLPPTAPKTIADYLIRPSKRLRPTSPAPAAAASAPLSSSSLSPE |
| (Japonica Group) | | QRRRADTNLALARARRHLRLAESKASGGTAKLEELLVEETWLE |
| | | ALPGELHKPYALELCRFVAHERLHSPVPVYPPPHLVFHALHATP |
| | | FDRVKAVIIGQDPYHGPGQAMGLSFSVPEGIKIPSSLANIFKELQK |
| | | DLGCTVPSHGNLERWAVQGVLMLNTVLTVREHQANSHAKKG |
| | | WEQFTDAVIKTISLKKSGIVFILWGNSAQAKTRLIDETKHHILKS |
| | | AHPSGLSASRGFFGCRHFSKTNQILERLGLSAIDWQL (SEQ ID |
| | | NO: 376) |
| Glycine max | XP_014619936.1 | MASAPSRTLTDFFQPASKRLKPTLPASCKSDDANASTLSVDQKL |
| | | RMEYNKLLAKSKRNLKLCVERVSKSKVQNAESGLGGVKLEELL |
| | | VEETWLEALPGELQKPYALTLSKFVESEISGGDGVIFPPTHLIFNA |
| | | LNSTPFHTVKAVILGQDPYHGPGQAMGLSFSVPEGIKVPSSLVNI |
| | | FKELHQDLGCSIPTHGNLQKWAVQGVLLLNAVLTVRKHQANSH |
| | | AKKGWEQFTDVVIKTISQKKEGVVFLLWGNSAREKSRLIDARK |
| | | HHVLTAAHPSGLSANRGFFGCRHFSRTNQLLEQMGIDPIDWQL |
| | | (SEQ ID NO: 377) |
| Glycine max | XP_006590826.2 | MGWKTFSNPLSDEANSSTLSLEQKSRVEYNKLLAKSKRNLKLCI |
| | | ERVSKSKESDSAGVKLEELLVEETWLDALPDELQKPYALTLSKF |
| | | VGSEISCGGDDVVFPPTHLIFNALNSTPFHTVKAVILGQVFGCLR |
| | | VQRDGNFDCIWVHFIAILRYNIRHVPDLCVGICLFTKIAARGMWI |
| | | IKCHFVYISLPAGLSIRKHQAYSHVKKGWEQFTDAVIKTISQKEG |
| | | VVFLLWGNSAPEKSRLIDATKHHVLTAAHPSGLSANRGFFGCRH |
| | | FSCTILEQMGIDPIDSQL(SEQ ID NO: 378) |
| Triticum | SPT20817.1 | MAPSPPTAPKTIADFFVRPAKRLRSGATTTTVVVPAASLSPSSGP |
| aestivum | | SDPTSLSPEQRRRADTNLALARARRNLRLAESKAAGGGAKLEEL |
| | | LVEETWLEALSGELRKPYALDLCRFVAHERLHAKVPVYPPPHL |
| | | VFHALHTTPFHSVKAVIIGQDPYHGPGQAMGLSFSVPEGIKIPSSL |
| | | QNIFKELHKDLGCTIPSHGNLERWAVQGILMLNTVLTVREHQAN |
| | | SHAKKGWEQFTDAVIKTVSQKKSGLVFILWGNSAQAKIRLIDET |
| | | KHHILKSAHPSGLSANRGFFGCRHFSQTNQILERLGLSTIDWQL |
| | | (SEQ ID NO: 379) |

### Deoxyuracil-Modified Guide RNA Inhibitors of Uracil-DNA Glycosylase

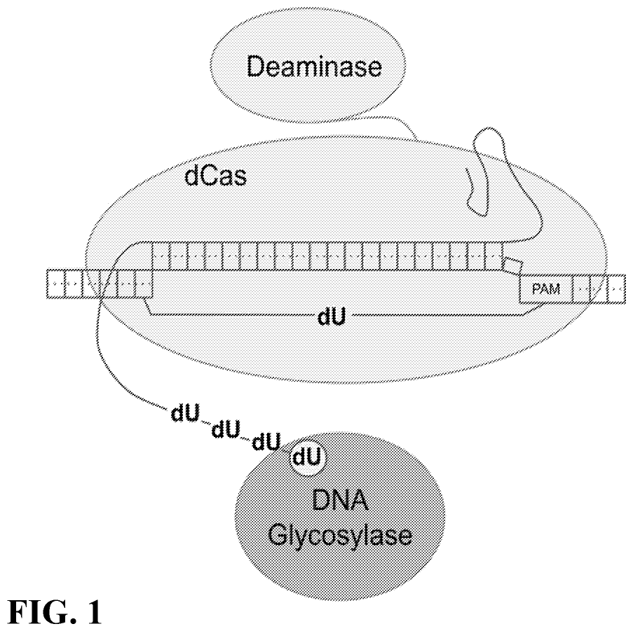

Several Cas family nucleases can accommodate 5′ or 3′ extensions to the spacer sequence, which may be engineered to contain additional natural or non-natural bases that lend novel functions to the base-editing system. As shown in FIG. 1, an extension including one or more deoxy-uracil (dU) residues can provide secondary substrates for uracil-DNA glycosylase, thereby reducing the likelihood that an exposed edited base will be bound and excised before being encoded by mismatch repair or replication machinery. The dU-extension can be further modified to resist glycosidic bond cleavage, enhance stability in vivo, or contain dU derivatives with enhanced ability to bind uracil-DNA glycosylase, all of which would produce more potent and longer-lived competitive inhibition of uracil-DNA glycosylase activity. Further, the uracil-containing extension may also be double-stranded (e.g., DNA) and contain one or more U:T, U:G, U:C, U:A, dU: T, dU: G, dU:C, or dU:A pairs. Useful dU analogs can include DNA lesions or repair pathway intermediates. For example, dU analogs are commercially available (from Sigma) in chemically synthesized oligos: 5-F-dU, 5-Br-dU, 5-I-dU, 2′-deoxypseudouridine, and 5,6-dihydro-dU. 2′-deoxypseudouridine is a non-cleavable substrate (Parikh et al., PNAS (2000) 97(10):5083-5088. doi:10.1073/pnas.97.10.5083).

Extension of the crRNA with dU bases can be accomplished during chemical synthesis or enzymatically using polymerases (Kent et al., eLife (2016) 5:e13740. PMID:27311885). The inhibitor-containing DNA oligonucleotides can be synthesized (e.g., by IDT or Sigma) by standard phosphoramidate chemistry, just like a PCR primer. Alternatively, poly-dU oligonucleotides of precise length and composition can be attached directly to a chemically-modified crRNA using one of a number of different attachment reagents. For example, using a “click” reaction (Lee et al., eLife (2017) 6:e25312. PMID: 28462777). Further, T4 RNA ligase 1 can be used to ligate a DNA oligonucleotide to single stranded RNA. Additional details regarding RNA guides including poly dU extensions may be found in Ahn et al., Sci Rep (2019) 9(1): 13911. PMID: 31227798; Bin Moon et al., Nat Commun (2018) 9(1):3651. PMID: 30194297.

The dU portion linked to a guide RNA can include nucleic acid analogs (e.g., analogs having other than a phosphodiester backbone). Nucleic acids can be purified from natural sources, produced using recombinant expression systems and optionally purified, chemically synthesized, etc. Where appropriate, e.g., in the case of chemically synthesized molecules, nucleic acids can include nucleoside analogs such as analogs having chemically modified bases or sugars, and backbone modifications. In some embodiments, a nucleic acid is or includes natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the present disclosure relates to RNA guides of Cas nucleases with poly dU (deoxy-Uracil) or dU-containing extensions. In some embodiments, the present disclosure relates to RNA guides of Cas nucleases with one or more dU (deoxy-Uracil) residues added to the tail of the RNA guides.

In some embodiments, the npUGI (e.g., an organochemical inhibitor of uracil glycosylase, a nucleic acid based inhibitor of uracil-DNA glycosylase, or a RNA guide of a dCas9 nuclease with a poly dU (deoxy-Uracil) extension) is linked to the base-editing system having the DNA binding domain (e.g., a Cas9 domain, a Cas12a domain, a Cas12h domain, a Cas12i domain, a CasX domain, a CasY domain, a C2c1 domain, a C2c2 domain, a C2c3 domain, a TALE domain, or a ZF domain) and the deaminase domain (e.g., an APOBEC1 deaminase domain, an APOBEC2 deaminase domain, an APOBEC3 deaminase domain, an APOBEC4 deaminase domain, an APOBEC3A deaminase domain, an APOBEC3B deaminase domain, an APOBEC3C deaminase domain, an APOBEC3D deaminase domain, an APOBEC3E deaminase domain, an APOBEC3F deaminase domain, an APOBEC3G deaminase domain, or an APOBEC3H deaminase domain). In some embodiments, the npUGI (e.g., e.g., an organochemical inhibitor of uracil glycosylase, a nucleic acid based inhibitor of uracil-DNA glycosylase, or a RNA guide of a dCas9 nuclease with a poly dU (deoxy-Uracil) extension) is not linked to the base-editing system having the DNA binding domain (e.g., a Cas9 domain, a Cas12a domain, a Cas12h domain, a Cas12i domain, a CasX domain, a CasY domain, a C2c1 domain, a C2c2 domain, a C2c3 domain, a TALE domain, or a ZF domain) and the deaminase domain (e.g., an APOBEC1 deaminase domain, an APOBEC2 deaminase domain, an APOBEC3 deaminase domain, an APOBEC4 deaminase domain, an APOBEC3A deaminase domain, an APOBEC3B deaminase domain, an APOBEC3C deaminase domain, an APOBEC3D deaminase domain, an APOBEC3E deaminase domain, an APOBEC3F deaminase domain, an APOBEC3G deaminase domain, or an APOBEC3H deaminase domain). In some embodiments, the base-editing system provided herein is generated in vitro by complexing. In some embodiments, the base-editing system provided herein is delivered by PEG-mediated transfection. In some embodiments, the base-editing system provided herein is generated in vitro by complexing a purified CRISPR-deaminase with the RNA-DNA hybrid guide RNA and is subsequently delivered by PEG-mediated transfection. In some embodiments, the base-editing system provided herein is vector-encoded. In some embodiments, the base-editing system provided herein is vector-encoded and co-delivered with exogenous nucleic acid via PEG-mediated transfection. In some embodiments, the base-editing system provided herein is delivered by PEG-mediated transfection while the small molecule inhibitor is delivered via cell culture media supplementation.

### Using Base-Editing Systems to Treat Diseases or Disorders

Significantly, 80-90% of protein mutations responsible for human disease arise from the substitution, deletion, or insertion of only a single nucleotide (Pan et al., Mol Biotechnol (2013) 55(1):54-62. PMID: 23089945). In some aspects, the present disclosure relates to using any of the base editors provided herein to treat a disease or disorder. For example, any of the base editors provided herein may be used to correct one or more mutations associated with any of the diseases or disorders provided herein. In some embodiments, the target DNA sequence includes a sequence associated with a disease or disorder. In some embodiments, the target DNA sequence includes a point mutation associated with a disease or disorder. In some embodiments, the activity of the DNA binding domain (e.g., a Cas9 domain, a Cas12a domain, a Cas12h domain, a Cas12i domain, a CasX domain, a CasY domain, a C2c1 domain, a C2c2 domain, a C2c3 domain, a TALE domain, or a ZF domain), the DNA binding base-editing system, or the complex results in a correction of the point mutation. In some embodiments, the target DNA sequence includes a T to C point mutation associated with a disease or disorder, wherein the deamination of the mutant C base results in a sequence that is not associated with a disease or disorder. In some embodiments, the target DNA sequence encodes a protein, the point mutation is in a codon, and the point mutation results in a change in the amino acid encoded by the mutant codon as compared to the wild-type codon. In some embodiments, the deamination of the mutant C results in a change of the amino acid encoded by the mutant codon. In some embodiments, the deamination of the mutant C results in the codon encoding the wild-type amino acid. In some embodiments, the base editor is used in vivo in a subject, e.g., a human subject. In some embodiments, the subject has a disease or disorder or has been diagnosed with a disease or disorder. In some embodiments, the disease or disorder is cystic fibrosis, phenylketonuria, epidermolytic hyperkeratosis (EHK), Charcot-Marie-Toot disease type 4J, neuroblastoma (B), von Willebrand disease (vWD), myotonia congenital, hereditary renal amyloidosis, dilated cardiomyopathy (DCM), hereditary lymphedema, familial Alzheimer's disease, HIV, Prion disease, chronic infantile neurologic cutaneous articular syndrome (CINCA), desmin-related myopathy (DRM), a neoplastic disease associated with a mutant PI3KCA protein, a mutant CTNNB 1 protein, a mutant HRAS protein, or a mutant p53 protein.

Some embodiments provide methods for using the base-editing system provided herein. In some embodiments, the base-editing system is used to introduce a point mutation into a nucleic acid by deaminating a target nucleobase, e.g., a C residue. In some embodiments, the deamination of the target nucleobase results in the correction of a genetic defect, e.g., in the correction of a point mutation that leads to a loss of function in a gene product. In some embodiments, the genetic defect is associated with a disease or disorder, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease or disorder. For example, in some embodiments, methods are provided herein that employ base-editing system to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

In some embodiments, the purpose of the methods provide herein is to restore the function of a dysfunctional gene via genome editing. The base-editing systems provided herein can be validated for gene editing-based human therapeutics in vitro, e.g., by correcting a disease-associated mutation in human cell culture. It will be understood by the skilled artisan that the base-editing systems provided herein, e.g., the base-editing systems including DNA binding domain (e.g., a Cas9 domain, a Cas12a domain, a Cas12h domain, a Cas12i domain, a CasX domain, a CasY domain, a C2c1 domain, a C2c2 domain, a C2c3 domain, an Argonaute domain, a TALE domain, or a ZF domain) and a nucleic acid deaminase domain can be used to correct any single pointT->C or A->G mutation. In the first case, deamination of the mutant C back to U corrects the mutation, and in the latter case, deamination of the C that is base-paired with the mutant G, followed by a round of replication, corrects the mutation.

An exemplary disease-relevant mutation that can be corrected by the provided base-editing systems in vitro or in vivo is the H1047R (A3140G) polymorphism in the PI3KCA protein. The phosphoinositide-3-kinase, catalytic alpha subunit (PI3KCA) protein acts to phosphorylate the 3-OH group of the inositol ring of phosphatidylinositol. The PI3KCA gene has been found to be mutated in many different carcinomas, and thus it is considered to be a potent oncogene. In fact, the A3 140G mutation is present in several NCI-60 cancer cell lines, such as, for example, the HCT116, SKOV3, and T47D cell lines, which are readily available from the American Type Culture Collection (ATCC).

In some embodiments, a cell carrying a mutation to be corrected, e.g., a cell carrying a point mutation, e.g., an A3 140G point mutation in exon 20 of the PI3KCA gene, resulting in a H1047R substitution in the PI3KCA protein, is contacted with an expression construct encoding a DNA binding domain (e.g., a Cas9 domain, a Cas12a domain, a Cas12h domain, a Cas12i domain, a CasX domain, a CasY domain, a C2c1 domain, a C2c2 domain, a C2c3 domain, an Argonaute domain, a TALE domain, or a ZF domain) deaminase base-editing system and an appropriately designed sgRNA targeting the base-editing system to the respective mutation site in the encoding PI3KCA gene. Control experiments can be performed where the sgRNAs are designed to target the base-editing systems to non-C residues that are within the PI3KCA gene. Genomic DNA of the treated cells can be extracted, and the relevant sequence of the PI3KCA genes PCR amplified and sequenced to assess the activities of the base-editing systems in human cell culture.

It will be understood that the example of correcting point mutations in PI3KCA is provided for illustration purposes and is not meant to limit the instant disclosure. The skilled artisan will understand that the instantly disclosed base-editing systems can be used to correct other point mutations and mutations associated with other cancers and with diseases other than cancer including other proliferative diseases.

The successful correction of point mutations in disease-associated genes and alleles opens up new strategies for gene correction with applications in therapeutics and basic research. Site-specific single-base modification systems like the disclosed base-editing systems of DNA binding (e.g., a Cas9 domain, a Cas12a domain, a Cas12h domain, a Cas12i domain, a CasX domain, a CasY domain, a C2c1 domain, a C2c2 domain, a C2c3 domain, an Argonaute domain, a TALE domain, or a ZF domain) and deaminase enzymes or domains also have applications in “reverse” gene therapy, where certain gene functions are purposely suppressed or abolished. In these cases, site-specifically mutating Tip (TGG), Gin (CAA and CAG), or Arg (CGA) residues to premature stop codons (TAA, TAG, TGA) can be used to abolish protein function in vitro, ex vivo, or in vivo.

The instant disclosure provides methods for the treatment of a subject diagnosed with a disease associated with or caused by a point mutation that can be corrected by a base-editing system provided herein. For example, in some embodiments, a method is provided that includes administering to a subject having such a disease, e.g., a cancer associated with a PI3KCA point mutation as described above, an effective amount of a DNA binding (e.g., a Cas9 domain, a Cas12a domain, a Cas12h domain, a Cas12i domain, a CasX domain, a CasY domain, a C2c1 domain, a C2c2 domain, a C2c3 domain, a TALE domain, or a ZF domain) deaminase base-editing system that corrects the point mutation or introduces a deactivating mutation into the disease-associated gene. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect.

The instant disclosure provides methods for the treatment of additional diseases or disorders, e.g., diseases or disorders that are associated or caused by a point mutation that can be corrected by deaminase-mediated gene editing. Some such diseases are described herein, and additional suitable diseases that can be treated with the strategies and base-editing systems provided herein will be apparent to those of skill in the art based on the instant disclosure. Exemplary suitable diseases and disorders are listed below. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues. Exemplary suitable diseases and disorders include, without limitation, cystic fibrosis (see, e.g., Schwank et al., Cell stem cell (2013) 13: 653-658; and Wu et al., Cell stem cell (2013) 13: 659-662); phenylketonuria, e.g., a phenylalanine to serine mutation at residue 835 (mouse) or 240 (human) or a homologous residue in phenylalanine hydroxylase gene (T to C mutation) (see, e.g., McDonald et al., Genomics (1997) 39:402-405); Bernard-Soulier syndrome (BSS), e.g., a phenylalanine to serine mutation at residue 55 or a homologous residue, or a cysteine to arginine mutation at residue 24 or a homologous residue in the platelet membrane glycoprotein IX (T to C mutation) (see, e.g., Noris et al., British Journal of Haematology (1997) 97: 312-320; and Ali et al., Hematol (2014) 93: 381-384); epidermolytic hyperkeratosis (EHK), e.g., a leucine to proline mutation at residue 160 or 161 (if counting the initiator methionine) or a homologous residue in keratin 1 (T to C mutation) (see, e.g., Chipev et al., Cell (1992) 70: 821-828; see also accession number P04264 in the UNIPROT database); chronic obstructive pulmonary disease (COPD), e.g., a leucine to proline mutation at residue 54 or 55 (if counting the initiator methionine) or a homologous residue in the processed form of α₁-antitrypsin or residue 78 in the unprocessed form or a homologous residue (T to C mutation) (see, e.g., Poller et al., Genomics (1993) 17: 740-743; see also accession number P01011 in the UNIPROT database); Charcot-Marie-Toot disease type 4J, e.g., a isoleucine to threonine mutation at residue 41 or a homologous residue (T to C mutation) (see, e.g., Lenk et al., PLoS Genetics (2011) 7: e1002104); neuroblastoma (NB), e.g., a leucine to proline mutation at residue 197 or a homologous residue in Caspase-9 (T to C mutation) (see, e.g., Kundu et al., 3 Biotech (2013) 3:225-234); von Willebrand disease (vWD), e.g., a cysteine to arginine mutation at residue 509 or a homologous residue in the processed form of von Willebrand factor, or at residue 1272 or a homologous residue in the unprocessed form of von Willebrand factor (T to C mutation) (see, e.g., Lavergne et al., Br J Haematol (1992) 82: 66-72; see also accession number P04275 in the UNIPROT database); myotonia congenital, e.g., a cysteine to arginine mutation at residue 277 or a homologous residue in the muscle chloride channel gene CLCN1 (T to C mutation) (see, e.g., Weinberger et al., The J of Physiology (2012) 590: 3449-3464); hereditary renal amyloidosis, e.g., a stop codon to arginine mutation at residue 78 or a homologous residue in the processed form of apolipoprotein A11 or at residue 101 or a homologous residue in the unprocessed form (T to C mutation) (see, e.g., Yazaki et al., Kidney Int (2003) 64: 11-16); dilated cardiomyopathy (DCM), e.g., a tryptophan to arginine mutation at residue 148 or a homologous residue in the FOXD4 gene (T to C mutation) (see, e.g., Minoretti et al., Int J of Mol Med (2007) 19: 369-372); hereditary lymphedema, e.g., a histidine to arginine mutation at residue 1035 or a homologous residue in VEGFR3 tyrosine kinase (A to G mutation) (see, e.g., Irrthum et al., Am J Hum Genet (2000) 67: 295-301); familial Alzheimer's disease, e.g., an isoleucine to valine mutation at residue 143 or a homologous residue in presenilin1 (A to G mutation) (see, e.g., Gallo et al., J Alzheimer's disease (2011) 25: 425-431); Prion disease, e.g., a methionine to valine mutation at residues 129 or a homologous residue in prion protein (A to G mutation) (see, e.g., Lewis et al., J of General Virology (2006) 87: 2443-2449); chronic infantile neurologic cutaneous articular syndrome (CINCA), e.g., a tyrosine to cysteine mutation at residue 570 or a homologous residue in cryopyrin (A to G mutation) (see, e.g., Fujisawa et al., Blood (2007) 109: 2903-2911); and desmin-related myopathy (DRM), e.g., an arginine to glycine mutation at residue 120 or a homologous residue in up crystallin (A to G mutation) (see, e.g., Kumar et al., J Biol Chem (1999) 274: 24137-24141).

Exemplary diseases or disorders that may be treated include, without limitation, 3-Methylglutaconic aciduria type 2, 46,XY gonadal dysgenesis, 4-Alpha-hydroxyphenylpyruvate hydroxylase deficiency, 6- pyruvoyl-tetrahydropterin synthase deficiency, achromatopsia, Acid-labile subunit deficiency, Acrodysostosis, acroerythrokeratoderma, ACTH resistance, ACTH-independent macronodular adrenal hyperplasia, Activated PI3K-delta syndrome, Acute intermittent porphyria, Acute myeloid leukemia, Adams-Oliver syndrome 1/5/6, Adenylosuccinate lyase deficiency, Adrenoleukodystrophy, Adult neuronal ceroid lipofuscinosis, Adult onset ataxia with oculomotor apraxia, Advanced sleep phase syndrome, Age-related macular degeneration, Alagille syndrome, Alexander disease, Allan-Herndon-Dudley syndrome, Alport syndrome, X-linked recessive, Alternating hemiplegia of childhood, Alveolar capillary dysplasia with misalignment of pulmonary veins, Amelogenesis imperfecta, Amyloidogenic transthyretin amyloidosis, Amyotrophic lateral sclerosis, Anemia (nonspherocytic hemolytic, due to G6PD deficiency), Anemia (sideroblastic, pyridoxine-refractory, autosomal recessive), Anonychia, Antithrombin III deficiency, Aortic aneurysm, Aplastic anemia, Apolipoprotein C2 deficiency, Apparent mineralocorticoid excess, Aromatase deficiency, Arrhythmogenic right ventricular cardiomyopathy, Familial hypertrophic cardiomyopathy, Hypertrophic cardiomyopathy, Arthrogryposis multiplex congenital, Aspartylglycosaminuria, Asphyxiating thoracic dystrophy, Ataxia with vitamin E deficiency, Ataxia (spastic), Atrial fibrillation, Atrial septal defect, atypical hemolytic-uremic syndrome, autosomal dominant CD11C+/CD1C+ dendritic cell deficiency, Autosomal dominant progressive external ophthalmoplegia with mitochondrial DNA deletions, Baraitser-Winter syndrome, Bartter syndrome, Basa ganglia calcification, Beckwith-Wiedemann syndrome, Benign familial neonatal seizures, Benign scapuloperoneal muscular dystrophy, Bernard Soulier syndrome, Beta thalassemia intermedia, Beta-D-mannosidosis, Bietti crystalline corneoretinal dystrophy, Bile acid malabsorption, Biotinidase deficiency, Borjeson-Forssman-Lehmann syndrome, Boucher Neuhauser syndrome, Bowen-Conradi syndrome, Brachydactyly, Brown-Vialetto-Van laere syndrome, Brugada syndrome, Cardiac arrhythmia, Cardiofaciocutaneous syndrome, Cardiomyopathy, Carnevale syndrome, Carnitine palmitoyltransferase II deficiency, Carpenter syndrome, Cataract, Catecholaminergic polymorphic ventricular tachycardia, Central core disease, Centromeric instability of chromosomes 1,9 and 16 and immunodeficiency, Cerebral autosomal dominant arteriopathy, Cerebro-oculo-facio-skeletal syndrome, Ceroid lipofuscinosis, Charcot-Marie-Tooth disease, Cholestanol storage disease, Chondrocalcinosis, Chondrodysplasia, Chronic progressive multiple sclerosis, Coenzyme Q10 deficiency, Cohen syndrome, Combined deficiency of factor V and factor VIII, Combined immunodeficiency, Combined oxidative phosphorylation deficiency, Combined partial 17-alpha-hydroxylase/17,20- lyase deficiency, Complement factor d deficiency, Complete combined 17- alpha-hydroxylase/17,20-lyase deficiency, Cone-rod dystrophy, Congenital contractural arachnodactyly, Congenital disorder of glycosylation, Congenital lipomatous overgrowth, Neoplasm of ovary, PIK3CA Related Overgrowth Spectrum, Congenital long QT syndrome, Congenital muscular dystrophy, Congenital muscular hypertrophy-cerebral syndrome, Congenital myasthenic syndrome, Congenital myopathy with fiber type disproportion, Eichsfeld type congenital muscular dystrophy, Congenital stationary night blindness, Corneal dystrophy, Cornelia de Lange syndrome, Craniometaphyseal dysplasia, Crigler Najjar syndrome, Crouzon syndrome, Cutis laxa with osteodystrophy, Cyanosis, Cystic fibrosis, Cystinosis, Cytochrome-c oxidase deficiency, Mitochondrial complex I deficiency, D-2-hydroxyglutaric aciduria, Danon disease, Deafness with labyrinthine aplasia microtia and microdontia (LAMM), Deafness, Deficiency of acetyl-CoA acetyltransferase, Deficiency of ferroxidase, Deficiency of UDPglucose-hexose-1-phosphate uridylyltransferase, Dejerine-Sottas disease, Desbuquois syndrome, DFNA, Diabetes mellitus type 2, Diabetes-deafness syndrome, Diamond-Blackfan anemia, Diastrophic dysplasia, Dihydropteridine reductase deficiency, Dihydropyrimidinase deficiency, Dilated cardiomyopathy, Disseminated atypical mycobacterial infection, Distal arthrogryposis, Distal hereditary motor neuronopathy, Donnai Barrow syndrome, Duchenne muscular dystrophy, Becker muscular dystrophy, Dyschromatosis universalis hereditaria, Dyskeratosis congenital, Dystonia, Early infantile epileptic encephalopathy, Ehlers-Danlos syndrome, Eichsfeld type congenital muscular dystrophy, Emery-Dreifuss muscular dystrophy, Enamel-renal syndrome, Epidermolysis bullosa dystrophica inversa, Epidermolysis bullosa herpetiformis, Epilepsy, Episodic ataxia, Erythrokeratodermia variabilis, Erythropoietic protoporphyria, Exercise intolerance, Exudative vitreoretinopathy, Fabry disease, Factor V deficiency, Factor VII deficiency, Factor xiii deficiency, Familial adenomatous polyposis, breast cancer, ovarian cancer, cold urticarial, chronic infantile neurological, cutaneous and articular syndrome, hemiplegic migraine, hypercholesterolemia, hypertrophic cardiomyopathy, hypoalphalipoproteinemia, hypokalemia-hypomagnesemia, juvenile gout, hyperlipoproteinemia, visceral amyloidosis, hypophosphatemic vitamin D refractory rickets, FG syndrome, Fibrosis of extraocular muscles, Finnish congenital nephrotic syndrome, focal epilepsy, Focal segmental glomerulosclerosis, Frontonasal dysplasia, Frontotemporal dementia, Fructose-biphosphatase deficiency, Gamstorp-Wohlfart syndrome, Ganglioside sialidase deficiency, GATA-1-related thrombocytopenia, Gaucher disease, Giant axonal neuropathy, Glanzmann thrombasthenia, Glomerulocystic kidney disease, Glomerulopathy, Glucocorticoid resistance, Glucose-6-phosphate transport defect, Glutaric aciduria, Glycogen storage disease, Gorlin syndrome, Holoprosencephaly, GRACILE syndrome, Haemorrhagic telangiectasia, Hemochromatosis, Hemoglobin H disease, Hemolytic anemia, Hemophagocytic lymphohistiocytosis, Carcinoma of colon, Myhre syndrome, leukoencephalopathy, Hereditary factor IX deficiency disease, Hereditary factor VIII deficiency disease, Hereditary factor XI deficiency disease, Hereditary fructosuria, Hereditary Nonpolyposis Colorectal Neoplasm, Hereditary pancreatitis, Hereditary pyropoikilocytosis, Elliptocytosis, Heterotaxy, Heterotopia, Histiocytic medullary reticulosis, Histiocytosis-lymphadenopathy plus syndrome, HNSHA due to aldolase A deficiency, Holocarboxylase synthetase deficiency, Homocysteinemia, Howel-Evans syndrome, Hydatidiform mole, Hypercalciuric hypercalcemia, Hyperimmunoglobulin D, Mevalonic aciduria, Hyperinsulinemic hypoglycemia, Hyperkalemic Periodic Paralysis, Paramyotonia congenita of von Eulenburg, Hyperlipoproteinemia, Hypermanganesemia, Hypermethioninemia, Hyperphosphatasemia, Hypertension, hypomagnesemia, Hypobetalipoproteinemia, Hypocalcemia, Hypogonadotropic hypogonadism, Hypogonadotropic hypogonadism, Hypohidrotic ectodermal dysplasia, Hyper-IgM immunodeficiency, Hypohidrotic X-linked ectodermal dysplasia, Hypomagnesemia, Hypoparathyroidism, Idiopathic fibrosing alveolitis, Immunodeficiency, Immunoglobulin A deficiency, Infantile hypophosphatasia, Infantile Parkinsonism-dystonia, Insulin-dependent diabetes mellitus, Intermediate maple syrup urine disease, Ischiopatellar dysplasia, Islet cell hyperplasia, Isolated growth hormone deficiency, Isolated lutropin deficiency, Isovaleric acidemia, Joubert syndrome, Juvenile polyposis syndrome, Juvenile retinoschisis, Kallmann syndrome, Kartagener syndrome, Kugelberg-Welander disease, Lattice corneal dystrophy, Leber congenital amaurosis, Leber optic atrophy, Left ventricular noncompaction, Leigh disease, Mitochondrial complex I deficiency, Leprechaunism syndrome, Arthrogryposis, Anterior horn cell disease, Leukocyte adhesion deficiency, Leukodystrophy, Leukoencephalopathy, Ovarioleukodystrophy, L-ferritin deficiency, Li-Fraumeni syndrome, Limb-girdle muscular dystrophy-dystroglycanopathy, Loeys-Dietz syndrome, Long QT syndrome, Macrocephaly/autism syndrome, Macular corneal dystrophy, Macular dystrophy, Malignant hyperthermia susceptibility, Malignant tumor of prostate, Maple syrup urine disease, Marden Walker like syndrome, Marfan syndrome, Marie Unna hereditary hypotrichosis, Mast cell disease, Meconium ileus, Medium-chain acyl-coenzyme A dehydrogenase deficiency, Melnick-Fraser syndrome, Mental retardation, Merosin deficient congenital muscular dystrophy, Mesothelioma, Metachromatic leukodystrophy, Metaphyseal chondrodysplasia, Methemoglobinemia, methylmalonic aciduria, homocystinuria, Microcephaly, chorioretinopathy, lymphedema, Microphthalmia, Mild non-PKU hyperphenylalanemia, Mitchell-Riley syndrome, mitochondrial 3-hydroxy-3-methylglutaryl-CoA synthase deficiency, Mitochondrial complex I deficiency, Mitochondrial complex III deficiency, Mitochondrial myopathy, Mucolipidosis III, Mucopolysaccharidosis, Multiple sulfatase deficiency, Myasthenic syndrome, Mycobacterium tuberculosis, Myeloperoxidase deficiency, Myhre syndrome, Myoclonic epilepsy, Myofibrillar myopathy, Myoglobinuria, Myopathy, Myopia, Myotonia congenital, Navajo neurohepatopathy, Nemaline myopathy, Neoplasm of stomach, Nephrogenic diabetes insipidus, Nephronophthisis, Nephrotic syndrome, Neurofibromatosis, Neutral lipid storage disease, Niemann-Pick disease, Non-ketotic hyperglycinemia, Noonan syndrome, Noonan syndrome-like disorder, Norum disease, Macular degeneration, N-terminal acetyltransferase deficiency, Oculocutaneous albinism, Oculodentodigital dysplasia, Ohdo syndrome, Optic nerve aplasia, Ornithine carbamoyltransferase deficiency, Orofaciodigital syndrome, Osteogenesis imperfecta, Osteopetrosis, Ovarian dysgenesis, Pachyonychia, Palmoplantar keratoderma, nonepidermolytic, Papillon-Lefevre syndrome, Haim-Munk syndrome, Periodontitis, Peeling skin syndrome, Pendred syndrome, Peroxisomal fatty acyl-coa reductase 1 disorder, Peroxisome biogenesis disorder, Pfeiffer syndrome, Phenylketonuria, Phenylketonuria, Hyperphenylalaninemia, non-PKU, Pituitary hormone deficiency, Pityriasis rubra pilaris, Polyarteritis nodosa, Polycystic kidney disease, Polycystic lipomembranous osteodysplasia, Polymicrogyria, Pontocerebellar hypoplasia, Porokeratosis, Posterior column ataxia, Primary erythromelalgia, hyperoxaluria, Progressive familial intrahepatic cholestasis, Progressive pseudorheumatoid dysplasia, Propionic acidemia, Pseudohermaphroditism, Pseudohypoaldosteronism, Pseudoxanthoma elasticum-like disorder, Purine-nucleoside phosphorylase deficiency, Pyridoxal 5-phosphate-dependent epilepsy, Renal dysplasia, retinal pigmentary dystrophy, cerebellar ataxia, skeletal dysplasia, Reticular dysgenesis, Retinitis pigmentosa, Usher syndrome, Retinoblastoma, Retinopathy, RRM2B-related mitochondrial disease, Rubinstein-Taybi syndrome, Schnyder crystalline corneal dystrophy, Sebaceous tumor, Severe congenital neutropenia, Severe myoclonic epilepsy in infancy, Severe X-linked myotubular myopathy, onychodysplasia, facial dysmorphism, hypotrichosis, Short-rib thoracic dysplasia, Sialic acid storage disease, Sialidosis, Sideroblastic anemia, Small fiber neuropathy, Smith-Magenis syndrome, Sorsby fundus dystrophy, Spastic ataxia, Spastic paraplegia, Spermatogenic failure, Spherocytosis, Sphingomyelin/cholesterol lipidosis, Spinocerebellar ataxia, Split-hand/foot malformation, Spondyloepimetaphyseal dysplasia, Platyspondylic lethal skeletal dysplasia, Squamous cell carcinoma of the head and neck, Stargardt disease, Sucrase-isomaltase deficiency, Sudden infant death syndrome, Supravalvar aortic stenosis, Surfactant metabolism dysfunction, Tangier disease, Tatton-Brown-rahman syndrome, Thoracic aortic aneurysms and aortic dissections, Thrombophilia, Thyroid hormone resistance, TNF receptor-associated periodic fever syndrome (TRAPS), Tooth agenesis, Torsades de pointes, Transposition of great arteries, Treacher Collins syndrome, Tuberous sclerosis syndrome, Tyrosinase-negative oculocutaneous albinism, Tyrosinase-positive oculocutaneous albinism, Tyrosinemia, UDPglucose-4-epimerase deficiency, Ullrich congenital muscular dystrophy, Bethlem myopathy Usher syndrome, UV-sensitive syndrome, Van der Woude syndrome, popliteal pterygium syndrome, Very long chain acyl-CoA dehydrogenase deficiency, Vesicoureteral reflux, Vitreoretinochoroidopathy, Von Hippel-Lindau syndrome, von Willebrand disease, Waardenburg syndrome, Warsaw breakage syndrome, WFSl-Related Disorders, Wilson disease, Xeroderma pigmentosum, X-linked agammaglobulinemia, X-linked hereditary motor and sensory neuropathy, X-linked severe combined immunodeficiency, and Zellweger syndrome.

In some embodiments, the base-editing system of the present disclosure targets a clinically relevant pathogenic gene variant with a point mutation (e.g., a pathogenic single nucleotide polymorphism (SNP). In some embodiments, the point mutation is a T to C or an A to G substitution. In some embodiments, the point mutation is located in or near a sequence placing the point mutation within the deamination activity window (e.g., near a NGG PAM). In some embodiments, the clinically relevant pathogenic gene variant with a point mutation is selected from the group of SEQ ID NO: 747, SEQ ID NO: 748, SEQ ID NO: 749, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766, SEQ ID NO: 767, SEQ ID NO: 768, SEQ ID NO: 769, SEQ ID NO: 770, SEQ ID NO: 771, SEQ ID NO: 772, SEQ ID NO: 773, SEQ ID NO: 774, SEQ ID NO: 775, SEQ ID NO: 776, SEQ ID NO: 777, SEQ ID NO: 778, SEQ ID NO: 779, SEQ ID NO: 780, SEQ ID NO: 781, SEQ ID NO: 782, SEQ ID NO: 783, SEQ ID NO: 784, SEQ ID NO: 785, SEQ ID NO: 786, SEQ ID NO: 787, SEQ ID NO: 788, SEQ ID NO: 789, SEQ ID NO: 790, SEQ ID NO: 791, SEQ ID NO: 792, SEQ ID NO: 793, SEQ ID NO: 794, SEQ ID NO: 795, SEQ ID NO: 796, SEQ ID NO: 797, SEQ ID NO: 798, SEQ ID NO: 799, SEQ ID NO: 800, SEQ ID NO: 801, SEQ ID NO: 802, SEQ ID NO: 803, SEQ ID NO: 804, SEQ ID NO: 805, SEQ ID NO: 806, SEQ ID NO: 807, SEQ ID NO: 808, SEQ ID NO: 809, SEQ ID NO: 810, SEQ ID NO: 811, SEQ ID NO: 812, SEQ ID NO: 813, SEQ ID NO: 814, SEQ ID NO: 815, SEQ ID NO: 816, SEQ ID NO: 817, SEQ ID NO: 818, SEQ ID NO: 819, SEQ ID NO: 820, SEQ ID NO: 821, SEQ ID NO: 822, SEQ ID NO: 823, SEQ ID NO: 824, SEQ ID NO: 825, SEQ ID NO: 826, SEQ ID NO: 827, SEQ ID NO: 828, SEQ ID NO: 829, SEQ ID NO: 830, SEQ ID NO: 831, SEQ ID NO: 832, SEQ ID NO: 833, SEQ ID NO: 834, SEQ ID NO: 835, SEQ ID NO: 836, SEQ ID NO: 837, SEQ ID NO: 838, SEQ ID NO: 839, SEQ ID NO: 840, SEQ ID NO: 841, SEQ ID NO: 842, SEQ ID NO: 843, SEQ ID NO: 844, SEQ ID NO: 845, SEQ ID NO: 846, SEQ ID NO: 847, SEQ ID NO: 848, SEQ ID NO: 849, SEQ ID NO: 850, SEQ ID NO: 851, SEQ ID NO: 852, SEQ ID NO: 853, SEQ ID NO: 854, SEQ ID NO: 855, SEQ ID NO: 856, SEQ ID NO: 857, SEQ ID NO: 858, SEQ ID NO: 859, SEQ ID NO: 860, SEQ ID NO: 861, SEQ ID NO: 862, SEQ ID NO: 863, SEQ ID NO: 864, SEQ ID NO: 865, SEQ ID NO: 866, SEQ ID NO: 867, SEQ ID NO: 868, SEQ ID NO: 869, SEQ ID NO: 870, SEQ ID NO: 871, SEQ ID NO: 872, SEQ ID NO: 873, SEQ ID NO: 874, SEQ ID NO: 875, SEQ ID NO: 876, SEQ ID NO: 877, SEQ ID NO: 878, SEQ ID NO: 879, SEQ ID NO: 880, SEQ ID NO: 881, SEQ ID NO: 882, SEQ ID NO: 883, SEQ ID NO: 884, SEQ ID NO: 885, SEQ ID NO: 886, SEQ ID NO: 887, SEQ ID NO: 888, SEQ ID NO: 889, SEQ ID NO: 890, SEQ ID NO: 891, SEQ ID NO: 892, SEQ ID NO: 893, SEQ ID NO: 894, SEQ ID NO: 895, SEQ ID NO: 896, SEQ ID NO: 897, SEQ ID NO: 898, SEQ ID NO: 899, SEQ ID NO: 900, SEQ ID NO: 901, SEQ ID NO: 902, SEQ ID NO: 903, SEQ ID NO: 904, SEQ ID NO: 905, SEQ ID NO: 906, SEQ ID NO: 907, SEQ ID NO: 908, SEQ ID NO: 909, SEQ ID NO: 910, SEQ ID NO: 911, SEQ ID NO: 912, SEQ ID NO: 913, SEQ ID NO: 914, SEQ ID NO: 915, SEQ ID NO: 916, SEQ ID NO: 917, SEQ ID NO: 918, SEQ ID NO: 919, SEQ ID NO: 920, SEQ ID NO: 921, SEQ ID NO: 922, SEQ ID NO: 923, SEQ ID NO: 924, SEQ ID NO: 925, SEQ ID NO: 926, SEQ ID NO: 927, SEQ ID NO: 928, SEQ ID NO: 929, SEQ ID NO: 930, SEQ ID NO: 931, SEQ ID NO: 932, SEQ ID NO: 933, SEQ ID NO: 934, SEQ ID NO: 935, SEQ ID NO: 936, SEQ ID NO: 937, SEQ ID NO: 938, SEQ ID NO: 939, SEQ ID NO: 940, SEQ ID NO: 941, SEQ ID NO: 942, SEQ ID NO: 943, SEQ ID NO: 944, SEQ ID NO: 945, SEQ ID NO: 946, SEQ ID NO: 947, SEQ ID NO: 948, SEQ ID NO: 949, SEQ ID NO: 950, SEQ ID NO: 951, SEQ ID NO: 952, SEQ ID NO: 953, SEQ ID NO: 954, SEQ ID NO: 955, SEQ ID NO: 956, SEQ ID NO: 957, SEQ ID NO: 958, SEQ ID NO: 959, SEQ ID NO: 960, SEQ ID NO: 961, SEQ ID NO: 962, SEQ ID NO: 963, SEQ ID NO: 964, SEQ ID NO: 965, SEQ ID NO: 966, SEQ ID NO: 967, SEQ ID NO: 968, SEQ ID NO: 969, SEQ ID NO: 970, SEQ ID NO: 971, SEQ ID NO: 972, SEQ ID NO: 973, SEQ ID NO: 974, SEQ ID NO: 975, SEQ ID NO: 976, SEQ ID NO: 977, SEQ ID NO: 978, SEQ ID NO: 979, SEQ ID NO: 980, SEQ ID NO: 981, SEQ ID NO: 982, SEQ ID NO: 983, SEQ ID NO: 984, SEQ ID NO: 985, SEQ ID NO: 986, SEQ ID NO: 987, SEQ ID NO: 988, SEQ ID NO: 989, SEQ ID NO: 990, SEQ ID NO: 991, SEQ ID NO: 992, SEQ ID NO: 993, SEQ ID NO: 994, SEQ ID NO: 995, SEQ ID NO: 996, SEQ ID NO: 997, SEQ ID NO: 998, SEQ ID NO: 999, SEQ ID NO: 1000, SEQ ID NO: 1001, SEQ ID NO: 1002, SEQ ID NO: 1003, SEQ ID NO: 1004, SEQ ID NO: 1005, SEQ ID NO: 1006, SEQ ID NO: 1007, SEQ ID NO: 1008, SEQ ID NO: 1009, SEQ ID NO: 1010, SEQ ID NO: 1011, SEQ ID NO: 1012, SEQ ID NO: 1013, SEQ ID NO: 1014, SEQ ID NO: 1015, SEQ ID NO: 1016, SEQ ID NO: 1017, SEQ ID NO: 1018, SEQ ID NO: 1019, SEQ ID NO: 1020, SEQ ID NO: 1021, SEQ ID NO: 1022, SEQ ID NO: 1023, SEQ ID NO: 1024, SEQ ID NO: 1025, SEQ ID NO: 1026, SEQ ID NO: 1027, SEQ ID NO: 1028, SEQ ID NO: 1029, SEQ ID NO: 1030, SEQ ID NO: 1031, SEQ ID NO: 1032, SEQ ID NO: 1033, SEQ ID NO: 1034, SEQ ID NO: 1035, SEQ ID NO: 1036, SEQ ID NO: 1037, SEQ ID NO: 1038, SEQ ID NO: 1039, SEQ ID NO: 1040, SEQ ID NO: 1041, SEQ ID NO: 1042, SEQ ID NO: 1043, SEQ ID NO: 1044, SEQ ID NO: 1045, SEQ ID NO: 1046, SEQ ID NO: 1047, SEQ ID NO: 1048, SEQ ID NO: 1049, SEQ ID NO: 1050, SEQ ID NO: 1051, SEQ ID NO: 1052, SEQ ID NO: 1053, SEQ ID NO: 1054, SEQ ID NO: 1055, SEQ ID NO: 1056, SEQ ID NO: 1057, SEQ ID NO: 1058, SEQ ID NO: 1059, SEQ ID NO: 1060, SEQ ID NO: 1061, SEQ ID NO: 1062, SEQ ID NO: 1063, SEQ ID NO: 1064, SEQ ID NO: 1065, SEQ ID NO: 1066, SEQ ID NO: 1067, SEQ ID NO: 1068, SEQ ID NO: 1069, SEQ ID NO: 1070, SEQ ID NO: 1071, SEQ ID NO: 1072, SEQ ID NO: 1073, SEQ ID NO: 1074, SEQ ID NO: 1075, SEQ ID NO: 1076, SEQ ID NO: 1077, SEQ ID NO: 1078, SEQ ID NO: 1079, SEQ ID NO: 1080, SEQ ID NO: 1081, SEQ ID NO: 1082, SEQ ID NO: 1083, SEQ ID NO: 1084, SEQ ID NO: 1085, SEQ ID NO: 1086, SEQ ID NO: 1087, SEQ ID NO: 1088, SEQ ID NO: 1089, SEQ ID NO: 1090, SEQ ID NO: 1091, SEQ ID NO: 1092, SEQ ID NO: 1093, SEQ ID NO: 1094, SEQ ID NO: 1095, SEQ ID NO: 1096, SEQ ID NO: 1097, SEQ ID NO: 1098, SEQ ID NO: 1099, SEQ ID NO: 1100, SEQ ID NO: 1101, SEQ ID NO: 1102, SEQ ID NO: 1103, SEQ ID NO: 1104, SEQ ID NO: 1105, SEQ ID NO: 1106, SEQ ID NO: 1107, SEQ ID NO: 1108, SEQ ID NO: 1109, SEQ ID NO: 1110, SEQ ID NO: 1111, SEQ ID NO: 1112, SEQ ID NO: 1113, SEQ ID NO: 1114, SEQ ID NO: 1115, SEQ ID NO: 1116, SEQ ID NO: 1117, SEQ ID NO: 1118, SEQ ID NO: 1119, SEQ ID NO: 1120, SEQ ID NO: 1121, SEQ ID NO: 1122, SEQ ID NO: 1123, SEQ ID NO: 1124, SEQ ID NO: 1125, SEQ ID NO: 1126, SEQ ID NO: 1127, SEQ ID NO: 1128, SEQ ID NO: 1129, SEQ ID NO: 1130, SEQ ID NO: 1131, SEQ ID NO: 1132, SEQ ID NO: 1133, SEQ ID NO: 1134, SEQ ID NO: 1135, SEQ ID NO: 1136, SEQ ID NO: 1137, SEQ ID NO: 1138, SEQ ID NO: 1139, SEQ ID NO: 1140, SEQ ID NO: 1141, SEQ ID NO: 1142, SEQ ID NO: 1143, SEQ ID NO: 1144, SEQ ID NO: 1145, SEQ ID NO: 1146, SEQ ID NO: 1147, SEQ ID NO: 1148, SEQ ID NO: 1149, SEQ ID NO: 1150, SEQ ID NO: 1151, SEQ ID NO: 1152, SEQ ID NO: 1153, SEQ ID NO: 1154, SEQ ID NO: 1155, SEQ ID NO: 1156, SEQ ID NO: 1157, SEQ ID NO: 1158, SEQ ID NO: 1159, SEQ ID NO: 1160, SEQ ID NO: 1161, SEQ ID NO: 1162, SEQ ID NO: 1163, SEQ ID NO: 1164, SEQ ID NO: 1165, SEQ ID NO: 1166, SEQ ID NO: 1167, SEQ ID NO: 1168, SEQ ID NO: 1169, SEQ ID NO: 1170, SEQ ID NO: 1171, SEQ ID NO: 1172, SEQ ID NO: 1173, SEQ ID NO: 1174, SEQ ID NO: 1175, SEQ ID NO: 1176, SEQ ID NO: 1177, SEQ ID NO: 1178, SEQ ID NO: 1179, SEQ ID NO: 1180, SEQ ID NO: 1181, SEQ ID NO: 1182, SEQ ID NO: 1183, SEQ ID NO: 1184, SEQ ID NO: 1185, SEQ ID NO: 1186, SEQ ID NO: 1187, SEQ ID NO: 1188, SEQ ID NO: 1189, SEQ ID NO: 1190, SEQ ID NO: 1191, SEQ ID NO: 1192, SEQ ID NO: 1193, SEQ ID NO: 1194, SEQ ID NO: 1195, SEQ ID NO: 1196, SEQ ID NO: 1197, SEQ ID NO: 1198, SEQ ID NO: 1199, SEQ ID NO: 1200, SEQ ID NO: 1201, SEQ ID NO: 1202, SEQ ID NO: 1203, SEQ ID NO: 1204, SEQ ID NO: 1205, SEQ ID NO: 1206, SEQ ID NO: 1207, SEQ ID NO: 1208, SEQ ID NO: 1209, SEQ ID NO: 1210, SEQ ID NO: 1211, SEQ ID NO: 1212, SEQ ID NO: 1213, SEQ ID NO: 1214, SEQ ID NO: 1215, SEQ ID NO: 1216, SEQ ID NO: 1217, SEQ ID NO: 1218, SEQ ID NO: 1219, SEQ ID NO: 1220, SEQ ID NO: 1221, SEQ ID NO: 1222, SEQ ID NO: 1223, SEQ ID NO: 1224, SEQ ID NO: 1225, SEQ ID NO: 1226, SEQ ID NO: 1227, SEQ ID NO: 1228, SEQ ID NO: 1229, SEQ ID NO: 1230, SEQ ID NO: 1231, SEQ ID NO: 1232, SEQ ID NO: 1233, SEQ ID NO: 1234, SEQ ID NO: 1235, SEQ ID NO: 1236, SEQ ID NO: 1237, SEQ ID NO: 1238, SEQ ID NO: 1239, SEQ ID NO: 1240, SEQ ID NO: 1241, SEQ ID NO: 1242, SEQ ID NO: 1243, SEQ ID NO: 1244, SEQ ID NO: 1245, SEQ ID NO: 1246, SEQ ID NO: 1247, SEQ ID NO: 1248, SEQ ID NO: 1249, SEQ ID NO: 1250, SEQ ID NO: 1251, SEQ ID NO: 1252, SEQ ID NO: 1253, SEQ ID NO: 1254, SEQ ID NO: 1255, SEQ ID NO: 1256, SEQ ID NO: 1257, SEQ ID NO: 1258, SEQ ID NO: 1259, SEQ ID NO: 1260, SEQ ID NO: 1261, SEQ ID NO: 1262, SEQ ID NO: 1263, SEQ ID NO: 1264, SEQ ID NO: 1265, SEQ ID NO: 1266, SEQ ID NO: 1267, SEQ ID NO: 1268, SEQ ID NO: 1269, SEQ ID NO: 1270, SEQ ID NO: 1271, SEQ ID NO: 1272, SEQ ID NO: 1273, SEQ ID NO: 1274, SEQ ID NO: 1275, SEQ ID NO: 1276, SEQ ID NO: 1277, SEQ ID NO: 1278, SEQ ID NO: 1279, SEQ ID NO: 1280, SEQ ID NO: 1281, SEQ ID NO: 1282, SEQ ID NO: 1283, SEQ ID NO: 1284, SEQ ID NO: 1285, SEQ ID NO: 1286, SEQ ID NO: 1287, SEQ ID NO: 1288, SEQ ID NO: 1289, SEQ ID NO: 1290, SEQ ID NO: 1291, SEQ ID NO: 1292, SEQ ID NO: 1293, SEQ ID NO: 1294, SEQ ID NO: 1295, SEQ ID NO: 1296, SEQ ID NO: 1297, SEQ ID NO: 1298, SEQ ID NO: 1299, SEQ ID NO: 1300, SEQ ID NO: 1301, SEQ ID NO: 1302, SEQ ID NO: 1303, SEQ ID NO: 1304, SEQ ID NO: 1305, SEQ ID NO: 1306, SEQ ID NO: 1307, SEQ ID NO: 1308, SEQ ID NO: 1309, SEQ ID NO: 1310, SEQ ID NO: 1311, SEQ ID NO: 1312, SEQ ID NO: 1313, SEQ ID NO: 1314, SEQ ID NO: 1315, SEQ ID NO: 1316, SEQ ID NO: 1317, SEQ ID NO: 1318, SEQ ID NO: 1319, SEQ ID NO: 1320, SEQ ID NO: 1321, SEQ ID NO: 1322, SEQ ID NO: 1323, SEQ ID NO: 1324, SEQ ID NO: 1325, SEQ ID NO: 1326, SEQ ID NO: 1327, SEQ ID NO: 1328, SEQ ID NO: 1329, SEQ ID NO: 1330, SEQ ID NO: 1331, SEQ ID NO: 1332, SEQ ID NO: 1333, SEQ ID NO: 1334, SEQ ID NO: 1335, SEQ ID NO: 1336, SEQ ID NO: 1337, SEQ ID NO: 1338, SEQ ID NO: 1339, SEQ ID NO: 1340, SEQ ID NO: 1341, SEQ ID NO: 1342, SEQ ID NO: 1343, SEQ ID NO: 1344, SEQ ID NO: 1345, SEQ ID NO: 1346, SEQ ID NO: 1347, SEQ ID NO: 1348, SEQ ID NO: 1349, SEQ ID NO: 1350, SEQ ID NO: 1351, SEQ ID NO: 1352, SEQ ID NO: 1353, SEQ ID NO: 1354, SEQ ID NO: 1355, SEQ ID NO: 1356, SEQ ID NO: 1357, SEQ ID NO: 1358, SEQ ID NO: 1359, SEQ ID NO: 1360, SEQ ID NO: 1361, SEQ ID NO: 1362, SEQ ID NO: 1363, SEQ ID NO: 1364, SEQ ID NO: 1365, SEQ ID NO: 1366, SEQ ID NO: 1367, SEQ ID NO: 1368, SEQ ID NO: 1369, SEQ ID NO: 1370, SEQ ID NO: 1371, SEQ ID NO: 1372, SEQ ID NO: 1373, SEQ ID NO: 1374, SEQ ID NO: 1375, SEQ ID NO: 1376, SEQ ID NO: 1377, SEQ ID NO: 1378, SEQ ID NO: 1379, SEQ ID NO: 1380, SEQ ID NO: 1381, SEQ ID NO: 1382, SEQ ID NO: 1383, SEQ ID NO: 1384, SEQ ID NO: 1385, SEQ ID NO: 1386, SEQ ID NO: 1387, SEQ ID NO: 1388, SEQ ID NO: 1389, SEQ ID NO: 1390, SEQ ID NO: 1391, SEQ ID NO: 1392, SEQ ID NO: 1393, SEQ ID NO: 1394, SEQ ID NO: 1395, SEQ ID NO: 1396, SEQ ID NO: 1397, SEQ ID NO: 1398, SEQ ID NO: 1399, SEQ ID NO: 1400, SEQ ID NO: 1401, SEQ ID NO: 1402, SEQ ID NO: 1403, SEQ ID NO: 1404, SEQ ID NO: 1405, SEQ ID NO: 1406, SEQ ID NO: 1407, SEQ ID NO: 1408, SEQ ID NO: 1409, SEQ ID NO: 1410, SEQ ID NO: 1411, SEQ ID NO: 1412, SEQ ID NO: 1413, SEQ ID NO: 1414, SEQ ID NO: 1415, SEQ ID NO: 1416, SEQ ID NO: 1417, SEQ ID NO: 1418, SEQ ID NO: 1419, SEQ ID NO: 1420, SEQ ID NO: 1421, SEQ ID NO: 1422, SEQ ID NO: 1423, SEQ ID NO: 1424, SEQ ID NO: 1425, SEQ ID NO: 1426, SEQ ID NO: 1427, SEQ ID NO: 1428, SEQ ID NO: 1429, SEQ ID NO: 1430, SEQ ID NO: 1431, SEQ ID NO: 1432, SEQ ID NO: 1433, SEQ ID NO: 1434, SEQ ID NO: 1435, SEQ ID NO: 1436, SEQ ID NO: 1437, SEQ ID NO: 1438, SEQ ID NO: 1439, SEQ ID NO: 1440, SEQ ID NO: 1441, SEQ ID NO: 1442, SEQ ID NO: 1443, SEQ ID NO: 1444, SEQ ID NO: 1445, SEQ ID NO: 1446, SEQ ID NO: 1447, SEQ ID NO: 1448, SEQ ID NO: 1449, SEQ ID NO: 1450, SEQ ID NO: 1451, SEQ ID NO: 1452, SEQ ID NO: 1453, SEQ ID NO: 1454, SEQ ID NO: 1455, SEQ ID NO: 1456, SEQ ID NO: 1457, SEQ ID NO: 1458, SEQ ID NO: 1459, SEQ ID NO: 1460, SEQ ID NO: 1461, SEQ ID NO: 1462, SEQ ID NO: 1463, SEQ ID NO: 1464, SEQ ID NO: 1465, SEQ ID NO: 1466, SEQ ID NO: 1467, SEQ ID NO: 1468, SEQ ID NO: 1469, SEQ ID NO: 1470, SEQ ID NO: 1471, SEQ ID NO: 1472, SEQ ID NO: 1473, SEQ ID NO: 1474, SEQ ID NO: 1475, SEQ ID NO: 1476, SEQ ID NO: 1477, SEQ ID NO: 1478, SEQ ID NO: 1479, SEQ ID NO: 1480, SEQ ID NO: 1481, SEQ ID NO: 1482, SEQ ID NO: 1483, SEQ ID NO: 1484, SEQ ID NO: 1485, SEQ ID NO: 1486, SEQ ID NO: 1487, SEQ ID NO: 1488, SEQ ID NO: 1489, SEQ ID NO: 1490, SEQ ID NO: 1491, SEQ ID NO: 1492, SEQ ID NO: 1493, SEQ ID NO: 1494, SEQ ID NO: 1495, SEQ ID NO: 1496, SEQ ID NO: 1497, SEQ ID NO: 1498, SEQ ID NO: 1499, SEQ ID NO: 1500, SEQ ID NO: 1501, SEQ ID NO: 1502, SEQ ID NO: 1503, SEQ ID NO: 1504, SEQ ID NO: 1505, SEQ ID NO: 1506, SEQ ID NO: 1507, SEQ ID NO: 1508, SEQ ID NO: 1509, SEQ ID NO: 1510, SEQ ID NO: 1511, SEQ ID NO: 1512, SEQ ID NO: 1513, SEQ ID NO: 1514, SEQ ID NO: 1515, SEQ ID NO: 1516, SEQ ID NO: 1517, SEQ ID NO: 1518, SEQ ID NO: 1519, SEQ ID NO: 1520, SEQ ID NO: 1521, SEQ ID NO: 1522, SEQ ID NO: 1523, SEQ ID NO: 1524, SEQ ID NO: 1525, SEQ ID NO: 1526, SEQ ID NO: 1527, SEQ ID NO: 1528, SEQ ID NO: 1529, SEQ ID NO: 1530, SEQ ID NO: 1531, SEQ ID NO: 1532, SEQ ID NO: 1533, SEQ ID NO: 1534, SEQ ID NO: 1535, SEQ ID NO: 1536, SEQ ID NO: 1537, SEQ ID NO: 1538, SEQ ID NO: 1539, SEQ ID NO: 1540, SEQ ID NO: 1541, SEQ ID NO: 1542, SEQ ID NO: 1543, SEQ ID NO: 1544, SEQ ID NO: 1545, SEQ ID NO: 1546, SEQ ID NO: 1547, SEQ ID NO: 1548, SEQ ID NO: 1549, SEQ ID NO: 1550, SEQ ID NO: 1551, SEQ ID NO: 1552, SEQ ID NO: 1553, SEQ ID NO: 1554, SEQ ID NO: 1555, SEQ ID NO: 1556, SEQ ID NO: 1557, SEQ ID NO: 1558, SEQ ID NO: 1559, SEQ ID NO: 1560, SEQ ID NO: 1561, SEQ ID NO: 1562, SEQ ID NO: 1563, SEQ ID NO: 1564, SEQ ID NO: 1565, SEQ ID NO: 1566, SEQ ID NO: 1567, SEQ ID NO: 1568, SEQ ID NO: 1569, SEQ ID NO: 1570, SEQ ID NO: 1571, SEQ ID NO: 1572, SEQ ID NO: 1573, SEQ ID NO: 1574, SEQ ID NO: 1575, SEQ ID NO: 1576, SEQ ID NO: 1577, SEQ ID NO: 1578, SEQ ID NO: 1579, SEQ ID NO: 1580, SEQ ID NO: 1581, SEQ ID NO: 1582, SEQ ID NO: 1583, SEQ ID NO: 1584, SEQ ID NO: 1585, SEQ ID NO: 1586, SEQ ID NO: 1587, SEQ ID NO: 1588, SEQ ID NO: 1589, SEQ ID NO: 1590, SEQ ID NO: 1591, SEQ ID NO: 1592, SEQ ID NO: 1593, SEQ ID NO: 1594, SEQ ID NO: 1595, SEQ ID NO: 1596, SEQ ID NO: 1597, SEQ ID NO: 1598, SEQ ID NO: 1599, SEQ ID NO: 1600, SEQ ID NO: 1601, SEQ ID NO: 1602, SEQ ID NO: 1603, SEQ ID NO: 1604, SEQ ID NO: 1605, SEQ ID NO: 1606, SEQ ID NO: 1607, SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 1616, SEQ ID NO: 1617, SEQ ID NO: 1618, SEQ ID NO: 1619, SEQ ID NO: 1620, SEQ ID NO: 1621, SEQ ID NO: 1622, SEQ ID NO: 1623, SEQ ID NO: 1624, SEQ ID NO: 1625, SEQ ID NO: 1626, SEQ ID NO: 1627, SEQ ID NO: 1628, SEQ ID NO: 1629, SEQ ID NO: 1630, SEQ ID NO: 1631, SEQ ID NO: 1632, SEQ ID NO: 1633, SEQ ID NO: 1634, SEQ ID NO: 1635, SEQ ID NO: 1636, SEQ ID NO: 1637, SEQ ID NO: 1638, SEQ ID NO: 1639, SEQ ID NO: 1640, SEQ ID NO: 1641, SEQ ID NO: 1642, SEQ ID NO: 1643, SEQ ID NO: 1644, SEQ ID NO: 1645, SEQ ID NO: 1646, SEQ ID NO: 1647, SEQ ID NO: 1648, SEQ ID NO: 1649, SEQ ID NO: 1650, SEQ ID NO: 1651, SEQ ID NO: 1652, SEQ ID NO: 1653, SEQ ID NO: 1654, SEQ ID NO: 1655, SEQ ID NO: 1656, SEQ ID NO: 1657, SEQ ID NO: 1658, SEQ ID NO: 1659, SEQ ID NO: 1660, SEQ ID NO: 1661, SEQ ID NO: 1662, SEQ ID NO: 1663, SEQ ID NO: 1664, SEQ ID NO: 1665, SEQ ID NO: 1666, SEQ ID NO: 1667, SEQ ID NO: 1668, SEQ ID NO: 1669, SEQ ID NO: 1670, SEQ ID NO: 1671, SEQ ID NO: 1672, SEQ ID NO: 1673, SEQ ID NO: 1674, SEQ ID NO: 1675, SEQ ID NO: 1676, SEQ ID NO: 1677, SEQ ID NO: 1678, SEQ ID NO: 1679, SEQ ID NO: 1680, SEQ ID NO: 1681, SEQ ID NO: 1682, SEQ ID NO: 1683, SEQ ID NO: 1684, SEQ ID NO: 1685, SEQ ID NO: 1686, SEQ ID NO: 1687, SEQ ID NO: 1688, SEQ ID NO: 1689, SEQ ID NO: 1690, SEQ ID NO: 1691, SEQ ID NO: 1692, SEQ ID NO: 1693, SEQ ID NO: 1694, SEQ ID NO: 1695, SEQ ID NO: 1696, SEQ ID NO: 1697, SEQ ID NO: 1698, SEQ ID NO: 1699, SEQ ID NO: 1700, SEQ ID NO: 1701, SEQ ID NO: 1702, SEQ ID NO: 1703, SEQ ID NO: 1704, SEQ ID NO: 1705, SEQ ID NO: 1706, SEQ ID NO: 1707, SEQ ID NO: 1708, SEQ ID NO: 1709, SEQ ID NO: 1710, SEQ ID NO: 1711, SEQ ID NO: 1712, SEQ ID NO: 1713, SEQ ID NO: 1714, SEQ ID NO: 1715, SEQ ID NO: 1716, SEQ ID NO: 1717, SEQ ID NO: 1718, SEQ ID NO: 1719, SEQ ID NO: 1720, SEQ ID NO: 1721, SEQ ID NO: 1722, SEQ ID NO: 1723, SEQ ID NO: 1724, SEQ ID NO: 1725, SEQ ID NO: 1726, SEQ ID NO: 1727, SEQ ID NO: 1728, SEQ ID NO: 1729, SEQ ID NO: 1730, SEQ ID NO: 1731, SEQ ID NO: 1732, SEQ ID NO: 1733, SEQ ID NO: 1734, SEQ ID NO: 1735, SEQ ID NO: 1736, SEQ ID NO: 1737, SEQ ID NO: 1738, SEQ ID NO: 1739, SEQ ID NO: 1740, SEQ ID NO: 1741, SEQ ID NO: 1742, SEQ ID NO: 1743, SEQ ID NO: 1744, SEQ ID NO: 1745, SEQ ID NO: 1746, SEQ ID NO: 1747, SEQ ID NO: 1748, SEQ ID NO: 1749, SEQ ID NO: 1750, SEQ ID NO: 1751, SEQ ID NO: 1752, SEQ ID NO: 1753, SEQ ID NO: 1754, SEQ ID NO: 1755, SEQ ID NO: 1756, SEQ ID NO: 1757, SEQ ID NO: 1758, SEQ ID NO: 1759, SEQ ID NO: 1760, SEQ ID NO: 1761, SEQ ID NO: 1762, SEQ ID NO: 1763, SEQ ID NO: 1764, SEQ ID NO: 1765, SEQ ID NO: 1766, SEQ ID NO: 1767, SEQ ID NO: 1768, SEQ ID NO: 1769, SEQ ID NO: 1770, SEQ ID NO: 1771, SEQ ID NO: 1772, SEQ ID NO: 1773, SEQ ID NO: 1774, SEQ ID NO: 1775, SEQ ID NO: 1776, SEQ ID NO: 1777, SEQ ID NO: 1778, SEQ ID NO: 1779, SEQ ID NO: 1780, SEQ ID NO: 1781, SEQ ID NO: 1782, SEQ ID NO: 1783, SEQ ID NO: 1784, SEQ ID NO: 1785, SEQ ID NO: 1786, SEQ ID NO: 1787, SEQ ID NO: 1788, SEQ ID NO: 1789, SEQ ID NO: 1790, SEQ ID NO: 1791, SEQ ID NO: 1792, SEQ ID NO: 1793, SEQ ID NO: 1794, SEQ ID NO: 1795, SEQ ID NO: 1796, SEQ ID NO: 1797, SEQ ID NO: 1798, SEQ ID NO: 1799, SEQ ID NO: 1800, SEQ ID NO: 1801, SEQ ID NO: 1802, SEQ ID NO: 1803, SEQ ID NO: 1804, SEQ ID NO: 1805, SEQ ID NO: 1806, SEQ ID NO: 1807, SEQ ID NO: 1808, SEQ ID NO: 1809, SEQ ID NO: 1810, SEQ ID NO: 1811, SEQ ID NO: 1812, SEQ ID NO: 1813, SEQ ID NO: 1814, SEQ ID NO: 1815, SEQ ID NO: 1816, SEQ ID NO: 1817, SEQ ID NO: 1818, SEQ ID NO: 1819, SEQ ID NO: 1820, SEQ ID NO: 1821, SEQ ID NO: 1822, SEQ ID NO: 1823, SEQ ID NO: 1824, SEQ ID NO: 1825, SEQ ID NO: 1826, SEQ ID NO: 1827, SEQ ID NO: 1828, SEQ ID NO: 1829, SEQ ID NO: 1830, SEQ ID NO: 1831, SEQ ID NO: 1832, SEQ ID NO: 1833, SEQ ID NO: 1834, SEQ ID NO: 1835, SEQ ID NO: 1836, SEQ ID NO: 1837, SEQ ID NO: 1838, SEQ ID NO: 1839, SEQ ID NO: 1840, SEQ ID NO: 1841, SEQ ID NO: 1842, SEQ ID NO: 1843, SEQ ID NO: 1844, SEQ ID NO: 1845, SEQ ID NO: 1846, SEQ ID NO: 1847, SEQ ID NO: 1848, SEQ ID NO: 1849, SEQ ID NO: 1850, SEQ ID NO: 1851, SEQ ID NO: 1852, SEQ ID NO: 1853, SEQ ID NO: 1854, SEQ ID NO: 1855, SEQ ID NO: 1856, SEQ ID NO: 1857, SEQ ID NO: 1858, SEQ ID NO: 1859, SEQ ID NO: 1860, SEQ ID NO: 1861, SEQ ID NO: 1862, SEQ ID NO: 1863, SEQ ID NO: 1864, SEQ ID NO: 1865, SEQ ID NO: 1866, SEQ ID NO: 1867, or SEQ ID NO: 1868. In some embodiments, the point mutation can be edited by the base-editing system of the present disclosure to produce a gene variant that is no longer pathogenic (e.g., no longer associated with the disease or disorder, no longer clinically relevant, wild-type, etc.).

### Using Base-Editing Systems in Plants

In some aspects, the disclosure relates to using the base-editing systems of the present disclosure in plants. Exemplary applications of the base-editing systems and methods of the present disclosure in plants may be to correct deleterious mutations, produce derivatives, modify sequences, insert sequences, and edit genomes of plants. These applications may be in coding sequences of plant genes or in non-coding sequences (e.g., regulatory sequences).

The base-editing systems disclosed herein may be used to produce derivatives of coding sequences. These derivatives may result in increased levels of specific (e.g., preselected) amino acids in the encoded polypeptide. In some embodiments, these derivatives are of agriculturally important genes or the derivatives modify genes to increase their agricultural importance. For example, the gene encoding the barley high lysine polypeptide (BHL) is derived from barley chymotrypsin inhibitor (see, e.g., U.S. application Ser. No. 08/740,682 and WO 98/20133). Other exemplary target proteins include methionine-rich plant proteins such as from sunflower seed (Lilley et al., Proceedings of the World Congress on Vegetable Protein Utilization in Human Foods and Animal Feedstuffs, ed. Applewhite (American Oil Chemists Society, Champaign, Ill.) (1989) pp. 497-502), corn (Pedersen et al., J Biol Chem (1986) 261:6279; and Kirihara et al., Gene (1988) 71:359), and rice (Musumura et al., Plant Mol. Biol. (1989) 12:123). Other agronomically important genes include latex, Floury 2, growth factors, seed storage factors, and transcription factors.

The base-editing systems disclosed herein can be used to modify herbicide resistance traits. Exemplary herbicide resistance traits include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene); glyphosate (e.g., the EPSPS gene and the GAT gene; see, e.g., U.S. Pat. App. Pub. No. 20040082770 and WO 03/092360); or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron. Additional herbicide resistance traits are for example described in U.S. Pat. App. Pub. No. 2016/0208243.

Sterility genes can also be modified using base-editing systems of the present disclosure. Modified sterility genes provide an alternative to physical sterility modifications, e.g., physical detasseling in corn. Examples of genes used in such ways include male tissue-preferred genes and genes with male sterility phenotypes such as QM, described in U.S. Pat. No. 5,583,210. Other sterility genes include kinases and those encoding compounds toxic to either male or female gametophytic development. Additional sterility traits are described for example in U.S. Pat. App. Pub. No. 2016/0208243.

Base-editing systems of the present disclosure can also be used to make haploid inducer lines as disclosed in WO2018086623.

Base-editing systems of the present disclosure can be used to alter agricultural product quality (e.g., to improve agricultural product quality, to alter agricultural product characteristics, etc.) or commercial traits (e.g., to improve commercial traits, to alter traits important for commercial production, etc.). For example, the quality of grain can be altered by modifying genes encoding traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389. Commercial traits can also be altered using base-editing systems of the present disclosure by modifying a gene that could increase, for example, starch for ethanol production, or provide expression of proteins. An exemplary important commercial use of modified plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al., J Bacteriol (1988) 170:5837-5847) facilitate expression of polyhyroxyalkanoates (PHAs).

In some embodiments, base-editing systems of the present disclosure can be used to alter exogenous products. Exogenous products include nucleic acids and proteins (e.g., enzymes or other protein-based products) that are not derived from the host organism species. Exogenous nucleic acids can be RNA, DNA, or RNA/DNA chimeras. Exogenous protein-based products can be from plants, prokaryotes, or other eukaryotes. Such protein products may include enzymes, cofactors, hormones, and the like. In some embodiments, the level of proteins, particularly modified proteins having improved amino acid distribution to improve the nutrient value of the plant, can be increased. In some embodiments, plants with improved nutrient value are achieved by the expression of such proteins having enhanced amino acid content.

The base-editing systems disclosed herein can also be used for insertion of heterologous genes and/or modification of native plant gene expression to achieve desirable plant traits. Such traits include, for example, disease resistance, herbicide tolerance, drought tolerance, salt tolerance, insect resistance, resistance against parasitic weeds, improved plant nutritional value, improved forage digestibility, increased grain yield, cytoplasmic male sterility, altered fruit ripening, increased storage life of plants or plant parts, reduced allergen production, and increased or decreased lignin content. Genes capable of conferring these desirable traits are disclosed in U.S. Pat. App. Pub. No. 2016/0208243.

The base-editing systems of the present disclosure may be used for base editing of any plant species, including, but not limited to, monocots and dicots (i.e., monocotyledonous and dicotyledonous, respectively). Examples of plant species of interest include, but are not limited to, corn (Zea mays), Brassica sp. (e.g., B. napus, B. rapa, B. juncea, particularly those Brassica species useful as sources of seed oil), alfalfa (Medicago sativa), rice (Oryza sativa), rye (Secale cereale), sorghum (Sorghum bicolor, Sorghum vulgare), oat (Avena sativa), barley (Hordeum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum) peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), and eucalyptus (Eucalyptus spp.). Additional plant species of interest may be classified as vegetables, ornamentals, or conifers.

### Base-Editing System Capacities and Methods of Using Base-Editing Systems

Some aspects of the disclosure are based on the recognition that any of the base-editing systems provided herein are capable of modifying a specific nucleotide base without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base-editing systems that efficiently modify (e.g., mutate or deaminate) a specific nucleotide within a nucleic acid, without generating a large number of insertions or deletions (i.e., indels) in the nucleic acid. In certain embodiments, any of the base-editing systems provided herein are capable of generating a greater proportion of intended modifications (e.g., point mutations or deaminations) versus indels. In some embodiments, the base-editing systems provided herein are capable of generating a ratio of intended point mutations to indels that is greater than 1:1. In some embodiments, the base-editing systems provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 8.5:1, at least 9:1, at least 9.5:1, at least 10:1, at least 11:1, at least 12:1, at least 13:1, at least 14:1, at least 15:1, at least 16:1, at least 17:1, at least 18:1, at least 19:1, at least 20:1, at least 25:1, at least 30:1, at least 35:1, at least 40:1, at least 45:1, at least 50:1, at least 60:1, at least 70:1, at least 80:1, at least 90:1, at least 100:1, at least 150:1, at least 200:1, at least 250:1, at least 300:1, at least 350:1, at least 400:1, at least 450:1, at least 500:1, at least 550:1, at least 600:1, at least 650:1, at least 700:1, at least 750:1, at least 800:1, at least 850:1, at least 900:1, at least 950:1, or at least 1000:1. The number of intended mutations and indels may be determined using any suitable method known in the art.

In some embodiments, the base-editing systems provided herein are capable of limiting formation of indels in a region of a nucleic acid. In some embodiments, the region is at a nucleotide targeted by a base-editing system or a region within 2, 3, 4, 5, 6, 7, 8, 9, or 10 nucleotides of a nucleotide targeted by a base-editing system. In some embodiments, any of the base-editing systems provided herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 5.5%, less than 6%, less than 6.5%, less than 7%, less than 7.5%, less than 8%, less than 8.5%, less than 9%, less than 9.5%, less than 10%, less than 11%, less than 12%, less than 13%, less than 14%, less than 15%, less than 16%, less than 17%, less than 18%, less than 19%, or less than 20%. The number of indels formed at a nucleic acid region may depend on the amount of time a nucleic acid (e.g., a nucleic acid within the genome of a cell) is exposed to a base-editing system. In some embodiments, an number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base-editing system.

Some aspects of the disclosure are based on the recognition that any of the base-editing systems provided herein are capable of efficiently generating an intended mutation, such as a point mutation, in a nucleic acid (e.g. a nucleic acid within a genome of a subject) without generating a significant number of unintended mutations, such as unintended point mutations. In some embodiments, an intended mutation is a mutation that is generated by a specific base-editing system bound to a gRNA, specifically designed to generate the intended mutation. In some embodiments, the intended mutation is a mutation associated with a disease or disorder. In some embodiments, the intended mutation is a cytosine (C) to thymine (T) point mutation associated with a disease or disorder. In some embodiments, the intended mutation is a guanine (G) to adenine (A) point mutation associated with a disease or disorder. In some embodiments, the intended mutation is a cytosine (C) to thymine (T) point mutation within the coding region of a gene. In some embodiments, the intended mutation is a guanine (G) to adenine (A) point mutation within the coding region of a gene. In some embodiments, the intended mutation is a point mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base-editing systems provided herein are capable of generating a ratio of intended mutations to unintended mutations (e.g., intended point mutations:unintended point mutations) that is greater than 1:1. In some embodiments, any of the base-editing systems provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 8.5:1, at least 9:1, at least 9.5:1, at least 10:1, at least 11:1, at least 12:1, at least 13:1, at least 14:1, at least 15:1, at least 16:1, at least 17:1, at least 18:1, at least 19:1, at least 20:1, at least 25:1, at least 30:1, at least 35:1, at least 40:1, at least 45:1, at least 50:1, at least 60:1, at least 70:1, at least 80:1, at least 90:1, at least 100:1, at least 150:1, at least 200:1, at least 250:1, at least 300:1, at least 350:1, at least 400:1, at least 450:1, at least 500:1, at least 550:1, at least 600:1, at least 650:1, at least 700:1, at least 750:1, at least 800:1, at least 850:1, at least 900:1, at least 950:1, or at least 1000:1. It should be appreciated that the characteristics of the base-editing systems described herein may be applied to any of the fusion proteins, or methods of using the fusion proteins provided herein.

Some aspects of the disclosure provide methods for editing a nucleic acid. In some embodiments, the method is a method for editing a nucleobase of a nucleic acid (e.g., a base pair of a double-stranded DNA sequence). In some embodiments, the method includes the steps of: a) contacting a target region of a nucleic acid (e.g., a double-stranded DNA sequence) with a complex including a base editor (e.g., a Cas9 domain fused to a cytidine deaminase domain) and a guide nucleic acid (e.g., gRNA), wherein the target region includes a targeted nucleobase pair, b) inducing strand separation of said target region, c) converting a first nucleobase of said target nucleobase pair in a single strand of the target region to a second nucleobase, and d) cutting no more than one strand of said target region, where a third nucleobase complementary to the first nucleobase base is replaced by a fourth nucleobase complementary to the second nucleobase; and the method results in less than 20% indel formation in the nucleic acid. It should be appreciated that in some embodiments, step b) is omitted. In some embodiments, the first nucleobase is a cytosine. In some embodiments, the second nucleobase is a deaminated cytosine, or a uracil. In some embodiments, the third nucleobase is a guanine. In some embodiments, the fourth nucleobase is an adenine. In some embodiments, the first nucleobase is a cytosine, the second nucleobase is a deaminated cytosine or a uracil, the third nucleobase is a guanine, and the fourth nucleobase is an adenine. In some embodiments, the method results in less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.5%, less than 0.2%, or less than 0.1% indel formation. In some embodiments, the method further includes replacing the second nucleobase with a fifth nucleobase that is complementary to the fourth nucleobase, thereby generating an intended edited base pair (e.g., C:G to T:A). In some embodiments, the fifth nucleobase is a thymine. In some embodiments, at least 5% of the intended basepairs are edited. In some embodiments, at least 10%, at least 15%, at least 20%, at least 25%, at least 30%, at least 35%, at least 40%, at least 45%, at least 50%, at least 55%, at least 60%, at least 65%, or at least 70% of the intended basepairs are edited.

Some aspects of the disclosure provide methods for editing a nucleic acid. In some embodiments, the method is a method for editing a nucleobase of a nucleic acid (e.g., a base pair of a double-stranded DNA sequence). In some embodiments, the method includes the steps of: a) contacting a target region of a nucleic acid (e.g., a double-stranded DNA sequence) with a complex including a base editor (e.g., a Cas12a domain fused to a cytidine deaminase domain) and a guide nucleic acid (e.g., gRNA), wherein the target region includes a targeted nucleobase pair, b) inducing strand separation of said target region, c) converting a first nucleobase of said target nucleobase pair in a single strand of the target region to a second nucleobase, and d) cutting no more than one strand of said target region, where a third nucleobase complementary to the first nucleobase base is replaced by a fourth nucleobase complementary to the second nucleobase; and the method results in less than 20% indel formation in the nucleic acid. It should be appreciated that in some embodiments, step b) is omitted. In some embodiments, the first nucleobase is a cytosine. In some embodiments, the second nucleobase is a deaminated cytosine, or a uracil. In some embodiments, the third nucleobase is a guanine. In some embodiments, the fourth nucleobase is an adenine. In some embodiments, the first nucleobase is a cytosine, the second nucleobase is a deaminated cytosine or a uracil, the third nucleobase is a guanine, and the fourth nucleobase is an adenine. In some embodiments, the method results in less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.5%, less than 0.2%, or less than 0.1% indel formation. In some embodiments, the method further includes replacing the second nucleobase with a fifth nucleobase that is complementary to the fourth nucleobase, thereby generating an intended edited base pair (e.g., C:G to T:A). In some embodiments, the fifth nucleobase is a thymine. In some embodiments, at least 5% of the intended basepairs are edited. In some embodiments, at least 10%, at least 15%, at least 20%, at least 25%, at least 30%, at least 35%, at least 40%, at least 45%, at least 50%, at least 55%, at least 60%, at least 65%, or at least 70% of the intended basepairs are edited.

Some aspects of the disclosure provide methods for editing a nucleic acid. In some embodiments, the method is a method for editing a nucleobase of a nucleic acid (e.g., a base pair of a double-stranded DNA sequence). In some embodiments, the method includes the steps of: a) contacting a target region of a nucleic acid (e.g., a double-stranded DNA sequence) with a complex including a base editor (e.g., a TALE domain fused to a cytidine deaminase domain) and a guide nucleic acid (e.g., gRNA), wherein the target region includes a targeted nucleobase pair, b) inducing strand separation of said target region, c) converting a first nucleobase of said target nucleobase pair in a single strand of the target region to a second nucleobase, and d) cutting no more than one strand of said target region, where a third nucleobase complementary to the first nucleobase base is replaced by a fourth nucleobase complementary to the second nucleobase; and the method results in less than 20% indel formation in the nucleic acid. It should be appreciated that in some embodiments, step b) is omitted. In some embodiments, the first nucleobase is a cytosine. In some embodiments, the second nucleobase is a deaminated cytosine, or a uracil. In some embodiments, the third nucleobase is a guanine. In some embodiments, the fourth nucleobase is an adenine. In some embodiments, the first nucleobase is a cytosine, the second nucleobase is a deaminated cytosine or a uracil, the third nucleobase is a guanine, and the fourth nucleobase is an adenine. In some embodiments, the method results in less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.5%, less than 0.2%, or less than 0.1% indel formation. In some embodiments, the method further includes replacing the second nucleobase with a fifth nucleobase that is complementary to the fourth nucleobase, thereby generating an intended edited base pair (e.g., C:G to T:A). In some embodiments, the fifth nucleobase is a thymine. In some embodiments, at least 5% of the intended basepairs are edited. In some embodiments, at least 10%, at least 15%, at least 20%, at least 25%, at least 30%, at least 35%, at least 40%, at least 45%, at least 50%, at least 55%, at least 60%, at least 65%, or at least 70% of the intended basepairs are edited.

Some aspects of the disclosure provide methods for editing a nucleic acid. In some embodiments, the method is a method for editing a nucleobase of a nucleic acid (e.g., a base pair of a double-stranded DNA sequence). In some embodiments, the method includes the steps of: a) contacting a target region of a nucleic acid (e.g., a double-stranded DNA sequence) with a complex including a base editor (e.g., a ZF domain fused to a cytidine deaminase domain) and a guide nucleic acid (e.g., gRNA), wherein the target region includes a targeted nucleobase pair, b) inducing strand separation of said target region, c) converting a first nucleobase of said target nucleobase pair in a single strand of the target region to a second nucleobase, and d) cutting no more than one strand of said target region, where a third nucleobase complementary to the first nucleobase base is replaced by a fourth nucleobase complementary to the second nucleobase; and the method results in less than 20% indel formation in the nucleic acid. It should be appreciated that in some embodiments, step b) is omitted. In some embodiments, the first nucleobase is a cytosine. In some embodiments, the second nucleobase is a deaminated cytosine, or a uracil. In some embodiments, the third nucleobase is a guanine. In some embodiments, the fourth nucleobase is an adenine. In some embodiments, the first nucleobase is a cytosine, the second nucleobase is a deaminated cytosine or a uracil, the third nucleobase is a guanine, and the fourth nucleobase is an adenine. In some embodiments, the method results in less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.5%, less than 0.2%, or less than 0.1% indel formation. In some embodiments, the method further includes replacing the second nucleobase with a fifth nucleobase that is complementary to the fourth nucleobase, thereby generating an intended edited base pair (e.g., C:G to T:A). In some embodiments, the fifth nucleobase is a thymine. In some embodiments, at least 5% of the intended basepairs are edited. In some embodiments, at least 10%, at least 15%, at least 20%, at least 25%, at least 30%, at least 35%, at least 40%, at least 45%, at least 50%, at least 55%, at least 60%, at least 65%, or at least 70% of the intended basepairs are edited.

In some embodiments, the ratio of intended products to unintended products in the target nucleotide is at least at least 10:1, at least 11:1, at least 12:1, at least 13:1, at least 14:1, at least 15:1, at least 16:1, at least 17:1, at least 18:1, at least 19:1, at least 20:1, at least 25:1, at least 30:1, at least 35:1, at least 40:1, at least 45:1, at least 50:1, at least 60:1, at least 70:1, at least 80:1, at least 90:1, at least 100:1, at least 150:1, or at least 200:1. In some embodiments, the ratio of intended point mutation to indel formation is greater than 1:1, greater than 10:1, greater than 50:1, greater than 100:1, greater than 500:1, or greater than 1000:1, or more. In some embodiments, the cut single strand (nicked strand) is hybridized to the guide nucleic acid. In some embodiments, the cut single strand is opposite to the strand including the first nucleobase. In some embodiments, the base-editing system includes a DNA binding domain (e.g., a Cas9 domain, a Cas12a domain, a Cas12h domain, a Cas12i domain, a CasX domain, a CasY domain, a C2c1 domain, a C2c2 domain, a C2c3 domain, a TALE domain, or a ZF domain). In some embodiments, the first base is cytosine, and the second base is not a G, C, A, or T. In some embodiments, the second base is uracil. In some embodiments, the first base is cytosine. In some embodiments, the second base is not a G, C, A, or T. In some embodiments, the second base is uracil. In some embodiments, the base-editing system inhibits base excision repair of the edited strand. In some embodiments, the base-editing system protects or binds the non-edited strand. In some embodiments, the base-editing system includes npUGI activity. In some embodiments, the base-editing system includes nickase activity. In some embodiments, the intended edited basepair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited basepair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream of the PAM site. In some embodiments, the method does not require a canonical (e.g., NGG) PAM site. In some embodiments, the nucleobase-editing system includes a linker. In some embodiments, the linker is in the range of 1-25 amino acids in length. In some embodiments, the linker is in the range of 5-20 amino acids in length. In some embodiments, the linker is 10, 11, 12, 13, 14, 15, 16, 17, 18, 19, or 20 amino acids in length. In some embodiments, the target region includes a target window, wherein the target window includes the target nucleobase pair. In some embodiments, the target window is in the range of 1-10 nucleotides. In some embodiments, the target window is in the range of 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window includes the intended edited base pair. In some embodiments, the method is performed using any of the base-editing systems provided herein. In some embodiments, the target window is a deamination window

In some embodiments, the disclosure provides methods for editing a nucleotide. In some embodiments, the disclosure provides a method for editing a nucleobase pair of a double-stranded DNA sequence. In some embodiments, the method includes a) contacting a target region of the double-stranded DNA sequence with a complex including a base-editing system and a guide nucleic acid (e.g., gRNA), where the target region includes a target nucleobase pair, b) inducing strand separation of said target region, c) converting a first nucleobase of said target nucleobase pair in a single strand of the target region to a second nucleobase, d) cutting no more than one strand of said target region, wherein a third nucleobase complementary to the first nucleobase base is replaced by a fourth nucleobase complementary to the second nucleobase, and the second nucleobase is replaced with a fifth nucleobase that is complementary to the fourth nucleobase, thereby generating an intended edited basepair, wherein the efficiency of generating the intended edited basepair is at least 5%. It should be appreciated that in some embodiments, step b is omitted. In some embodiments, at least 5% of the intended basepairs are edited. In some embodiments, at least 10%, at least 15%, at least 20%, at least 25%, at least 30%, at least 35%, at least 40%, at least 45%, at least 50%, at least 55%, at least 60%, at least 65%, or at least 70% of the intended basepairs are edited. In some embodiments, the method causes less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.5%, less than 0.2%, or less than 0.1% indel formation. In some embodiments, the ratio of intended product to unintended products at the target nucleotide is at least at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 8.5:1, at least 9:1, at least 9.5:1, at least 10:1, at least 11:1, at least 12:1, at least 13:1, at least 14:1, at least 15:1, at least 16:1, at least 17:1, at least 18:1, at least 19:1, at least 20:1, at least 25:1, at least 30:1, at least 35:1, at least 40:1, at least 45:1, at least 50:1, at least 60:1, at least 70:1, at least 80:1, at least 90:1, at least 100:1, at least 150:1, or at least 200:1. In some embodiments, the ratio of intended point mutation to indel formation is greater than 1:1, 10:1, 50:1, 100:1, 500:1, or 1000:1. In some embodiments, the cut single strand is hybridized to the guide nucleic acid. In some embodiments, the cut single strand is opposite to the strand including the first nucleobase. In some embodiments, the first base is cytosine. In some embodiments, the second nucleobase is not G, C, A, or T. In some embodiments, the second base is uracil. In some embodiments, the base-editing system inhibits base excision repair of the edited strand. In some embodiments, the base-editing system protects or binds the non-edited strand. In some embodiments, the base-editing system includes npUGI activity. In some embodiments, the nucleobase edit includes nickase activity. In some embodiments, the intended edited basepair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited basepair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the base-editing system includes a linker. In some embodiments, the linker is in the range of 1-25 amino acids in length. In some embodiments, the linker is in the range of 5-20 amino acids in length. In some embodiments, the linker is 10, 11, 12, 13, 14, 15, 16, 17, 18, 19, or 20 amino acids in length. In some embodiments, the target region includes a target window, wherein the target window includes the target nucleobase pair. In some embodiments, the target window is in the range of 1-10 nucleotides. In some embodiments, the target window is in the range of 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair occurs within the target window. In some embodiments, the target window includes the intended edited base pair. In some embodiments, the base-editing system is any one of the base-editing systems provided herein.

### Kits, Vectors, and Cells

Some aspects of this disclosure provide kits including a nucleic acid construct, including (a) a nucleotide sequence encoding a Cas9 protein, a Cas12a protein, a Cas12h protein, a Cas12i protein, a CasX protein, a CasY protein, a C2c1 protein, a C2c2 protein, a C2c3 protein, a TALE protein, or a ZF protein or a Cas9 base-editing system, a Cas12a base-editing system, a Cas12h base-editing system, a Cas12i base-editing system, a CasX base-editing system, a CasY base-editing system, a C2c1 base-editing system, a C2c2 base-editing system, a C2c3 base-editing system, an Argonaute base-editing system, a TALE base-editing system, or a ZF base-editing system as provided herein; and (b) a heterologous promoter that drives expression of the sequence of (a). In some embodiments, the promoter is inducible, tissue-specific, or organism specific. In some embodiments, the kit further includes an expression construct encoding a guide RNA backbone, wherein the construct includes a cloning site positioned to allow the cloning of a nucleic acid sequence identical or complementary to a target sequence into the guide RNA backbone.

Some aspects of this disclosure provide polynucleotides encoding a Cas9 protein, a Cas12a protein, a Cas12h protein, a Cas12i protein, a CasX protein, a CasY protein, a C2c1 protein, a C2c2 protein, a C2c3 protein, an Argonaute protein, a TALE protein, or a ZF protein or a Cas9 base-editing system, a Cas12a base-editing system, a Cas12h base-editing system, a Cas12i base-editing system, a CasX base-editing system, a CasY base-editing system, a C2c1 base-editing system, a C2c2 base-editing system, a C2c3 base-editing system, an Argonaute base-editing system, a TALE base-editing system, or a ZF base-editing system as provided herein. Some aspects of this disclosure provide vectors including such polynucleotides. In some embodiments, the vector includes a heterologous promoter driving expression of the polynucleotide. In some embodiments, the vector is delivered by PEG-mediated transfection.

Some aspects of this disclosure provide cells including a Cas9 protein, a Cas12a protein, a Cas12h protein, a Cas12i protein, a CasX protein, a CasY protein, a C2c1 protein, a C2c2 protein, a C2c3 protein, an Argonaute protein, a TALE protein, or a ZF protein or a Cas9 base-editing system, a Cas12a base-editing system, a Cas12h base-editing system, a Cas12i base-editing system, a CasX base-editing system, a CasY base-editing system, a C2c1 base-editing system, a C2c2 base-editing system, a C2c3 base-editing system, an Argonaute base-editing system, a TALE base-editing system, or a ZF base-editing system, a nucleic acid molecule encoding the DNA binding base-editing system, a complex include a Cas9 protein, a Cas12a protein, a Cas12h protein, a Cas12i protein, a CasX protein, a CasY protein, a C2c1 protein, a C2c2 protein, a C2c3 protein, an Argonaute protein, a TALE protein, or a ZF protein and the gRNA, and/or a vector as provided herein.

The description of exemplary embodiments of the base-editing systems above is provided for illustration purposes only and not meant to be limiting. Additional base-editing systems, e.g., variations of the exemplary systems described in detail above, are also embraced by this disclosure.

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4 ed., Cold Spring Harbor Laboratory Press, Cold Spring Harbor, N.Y. (2012)).

The terms “nucleic acid” and “nucleic acid molecule,” as used herein, refer to a compound including a nucleobase and an acidic moiety, e.g., a nucleoside, a nucleotide, or a polymer of nucleotides. Typically, polymeric nucleic acids, e.g., nucleic acid molecules including three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain including three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or portion thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can include nucleoside analogs such as analogs having chemically modified bases or sugars, and backbone modifications. A nucleic acid sequence is presented in the 5′ to 3′ direction unless otherwise indicated. In some embodiments, a nucleic acid is or includes natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nucleic acid editing domain,” as used herein refers to a protein or enzyme capable of making one or more modifications (e.g., deamination of a cytidine residue) to a nucleic acid (e.g., DNA or RNA). Exemplary nucleic acid editing domains include, but are not limited to a deaminase, a nuclease, a nickase, a recombinase, a methyltransferase, a methylase, an acetylase, an acetyltransferase, a transcriptional activator, or a transcriptional repressor domain. In some embodiments the nucleic acid editing domain is a deaminase (e.g., a cytidine deaminase, such as an APOBEC or an AID deaminase).

The term “effective amount,” as used herein, refers to an amount of a biologically active agent that is sufficient to elicit a desired biological response. For example, in some embodiments, an effective amount of a nuclease may refer to the amount of the nuclease that is sufficient to induce cleavage of a target site specifically bound and cleaved by the nuclease. In some embodiments, an effective amount of a base-editing system provided herein, e.g., of a base-editing system including a nuclease inactive Cas domain (e.g., a nuclease inactive Cas9 domain or a nuclease inactive Cas12a domain) and a nucleic acid editing domain (e.g., a deaminase domain) may refer to the amount of the base-editing system that is sufficient to induce editing of a target site specifically bound and edited by the base-editing system. As will be appreciated by the skilled artisan, the effective amount of an agent, e.g., a base-editing system, a nuclease, a deaminase, a recombinase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

The term “linker,” as used herein, refers to a chemical group or a molecule linking two molecules or moieties, e.g., two domains of a base-editing system, such as, for example, a nuclease inactive Cas domain (e.g., a nuclease inactive Cas9 domain or a nuclease inactive Cas12a domain) and a nucleic acid editing domain (e.g., a deaminase domain). In some embodiments, a linker joins a gRNA binding domain of an RNA-programmable nuclease, including a Cas nuclease domain (e.g., a Cas9 nuclease domain or a Cas12a nuclease domain), and the catalytic domain of a nucleic-acid editing protein. In some embodiments, a linker joins a dCas domain (e.g., a dCas9 domain or a dCas12a domain) and a nucleic-acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is in the range of 5-100 amino acids in length. In some embodiments, the linker is 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The term “proliferative disease,” as used herein, refers to any disease in which cell or tissue homeostasis is disturbed in that a cell or cell population exhibits an abnormally elevated proliferation rate. Proliferative diseases include hyperproliferative diseases, such as pre-neoplastic hyperplastic conditions and neoplastic diseases. Neoplastic diseases are characterized by an abnormal proliferation of cells and include both benign and malignant neoplasias. Malignant neoplasia is also referred to as cancer.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a portion of a naturally occurring protein or peptide. A protein, peptide, or polypeptide may be naturally occurring, recombinant, or synthetic, or any combination thereof. The term “base-editing system” as used herein refers to a hybrid molecule which includes protein domains from at least two different proteins, and non-protein domains that may or may not be chemically linked to the hybrid molecule. One protein may be located at the amino-terminal (N-terminal) portion of the base-editing system or at the carboxy-terminal (C-terminal) protein thus forming an “amino-terminal base-editing system” or a “carboxy-terminal base-editing system,” respectively. A base-editing system may include different domains, for example, a nucleic acid binding domain (e.g., the gRNA binding domain of Cas9 that directs the binding of the protein to a target site) and a nucleic acid cleavage domain or a catalytic domain of a nucleic-acid editing protein. In some embodiments, a base-editing system includes a proteinaceous part, e.g., an amino acid sequence constituting a nucleic acid binding domain, and an organic compound, e.g., a compound that can act as a nucleic acid cleavage agent. In some embodiments, a base-editing system is in a complex with, or is in association with, a nucleic acid, e.g., RNA. Any of the base-editing systems provided herein may be produced by any method known in the art. For example, the base-editing systems provided herein may be produced via recombinant expression and purification, which is especially suited for base-editing systems including a peptide linker. Methods for recombinant expression and purification are well known, and include those described by Green and Sambrook, Molecular Cloning: A Laboratory Manual (4th ed., Cold Spring Harbor Laboratory Press, Cold Spring Harbor, N.Y. (2012)).

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

The term “target site” refers to a sequence within a nucleic acid molecule that is deaminated by a deaminase or a base-editing system including a deaminase, (e.g., a dCas9-deaminase base-editing system provided herein).

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “recombinant” as used herein in the context of proteins or nucleic acids refers to proteins or nucleic acids that do not occur in nature, but are the product of human engineering. For example, in some embodiments, a recombinant protein or nucleic acid molecule includes an amino acid or nucleotide sequence that includes at least one, at least two, at least three, at least four, at least five, at least six, or at least seven mutations as compared to any naturally occurring sequence.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

Having generally described this invention, the same will be better understood by reference to certain specific examples, which are included herein to further illustrate the invention and are not intended to limit the scope of the invention as defined by the claims.

### EXAMPLES

The present disclosure is described in further detail in the following examples which are not in any way intended to limit the scope of the disclosure as claimed. The following examples are offered to illustrate, but not to limit the claimed disclosure.

### Example 1: Deoxyuracil-Modified Guide RNA Inhibition of Uracil-DNA Glycosylase

### Materials and Methods

A Cas12a fully processed crRNA sequence is chemically synthesized (Integrated DNA Technologies, Coralville, Iowa, USA) and encoded with the ‘spacer’ sequence ‘TTCAGGAGAAGCATGGTTCTA’ (SEQ ID NO: 384). The spacer sequence targets the Zea mays phytoene desaturase (PDS) gene (LOC542329, SEQ ID NO: 382) at base C3488. This targeting mutates the wild-type PDS amino acid sequence (SEQ ID NO: 383) to yield a protein with the amino acid mutation H298Y (SEQ ID NO: 386). The Z. mays inbred B73 is used.

The 5′-phosphorylated, uracil-containing DNA oligonucleotide ‘Phos-AUCUAUCUAUCUAUCUAUCU’ (SEQ ID NO: 385) is chemically synthesized (Integrated DNA Technologies, Coralville, Iowa, USA). The RNA and DNA oligonucleotides are mixed and ligated into a single RNA-DNA hybrid using T4 RNA Ligase 1 (New England Biolabs, Ipswich, Mass., USA).

A cytosine deaminase base editing system is generated in vitro by complexing a purified CRISPR-deaminase (cytosine base editor) with the RNA-DNA hybrid guide RNA, of protein and guide RNA(s) targeting the B73 Z. mays PDS gene (LOC542329, SEQ ID NO: 382) is subsequently delivered by PEG-mediated transfection.

### Results

The Z. mays PDS gene will be edited at C3488. The Z. mays PDS protein will include the amino acid mutation H298Y (i.e., will be SEQ ID NO: 386).

### Example 2: Nucleic Acid Inhibition of Uracil-DNA Glycosylase

### Materials and Methods

A short, double-stranded RNA is synthesized to target the 21-nt sequence AAATACCTTCTAGCTTAGGTA (SEQ ID NO: 381) of the Zea mays uracil-DNA glycosylase mRNA transcript (SEQ ID NO: 380). This sequence is synthesized with well-characterized structural elements for binding to the endogenous AGO RNA-silencing machinery. The Z. mays inbred B73 is used.

Exogenous RNA is co-delivered to Z. mays mid-leaf protoplasts using PEG-mediated transfection along with a vector-encoded cytosine base editor (e.g., the fusion protein of the base-editing system) driven by the ZmUBI1 promoter and guide RNA specific to the B73 Z. mays PDS gene (LOC542329, SEQ ID NO: 382).

### Results

The Z. mays PDS gene will be edited at C3488. The Z. mays PDS protein will include the amino acid mutation H298Y (i.e., will be SEQ ID NO: 386).

### Example 3: Small Molecule Inhibition of Uracil-DNA Glycosylase

### Materials and Methods

Isolated mid-leaf protoplasts from Zea mays are cultured in media supplemented with 1-methoxyethyl-6-(p-n-octylanilino) uracil (CID: 129848789) or related compound(s) (see section “Small molecule inhibitors of uracil-DNA glycosylase”). The Z. mays inbred B73 is used.

A cytosine deaminase base editing system composed of protein and guide RNA(s) targeting the B73 Z. mays PDS gene (LOC542329, SEQ ID NO: 382) is subsequently delivered by PEG-mediated transfection.

### Results

The Z. mays PDS gene will be edited at C3488. The Z. mays PDS protein will include the amino acid mutation H298Y (i.e., will be SEQ ID NO: 386).

### Example 4: Deoxyuracil-Modified Guide RNA, Nucleic Acid Knockdown, or Small Molecule Inhibition of Uracil-DNA Glycosylase in Glycine Max

### Materials and Methods

A Cas12a fully processed crRNA sequence is chemically synthesized (Integrated DNA Technologies, Coralville, Iowa, USA) encoded with the ‘spacer’ sequence, which targets the Glycine max phytoene desaturase (PDS) to yield an amino acid mutation.

The 5′-phosphorylated, uracil-containing DNA a oligonucleotide is chemically synthesized (Integrated DNA Technologies, Coralville, Iowa, USA). The RNA and DNA oligonucleotides are mixed and ligated into a single RNA-DNA hybrid using T4 RNA Ligase 1 (New England Biolabs, Ipswich, NIA, USA). A cytosine deaminase base editing system is generated in vitro by complexing a purified CRISPR-deaminase (cytosine base editor) with the RNA-DNA hybrid guide RNA, of protein and guide RNA(s) targeting the G. max PDS gene is subsequently delivered by PEG-mediated transfection.

Alternatively, a short, double-stranded RNA is synthesized to target a 21-nt sequence of the G. max uracil-DNA glycosylase mRNA transcript. This sequence is synthesized with well-characterized structural elements for binding to the endogenous AGO RNA-silencing machinery. Exogenous RNA is co-delivered to G. max mid-leaf protoplasts using PEG-mediated transfection along with a vector-encoded cytosine base editor (e.g., the fusion protein of the base-editing system) driven by a promoter and guide RNA specific to the G. max PDS gene.

In yet another alternative, isolated mid-leaf protoplasts from G. max are cultured in media supplemented with 1-methoxyethyl-6-(p-n-octylanilino) uracil (CID: 129848789) or related compound(s) (see section “Small molecule inhibitors of uracil-DNA glycosylase”). A cytosine deaminase base editing system composed of protein and guide RNA(s) targeting the G. max PDS gene is subsequently delivered by PEG-mediated transfection.

### Results

The G. max PDS gene will be edited. The G. max PDS protein will include a targeted amino acid alteration (e.g., a single amino acid change, a point mutation, etc.).

1. A base-editing system comprising
(i) a fusion protein comprising (a) a DNA-binding domain and (b) a cytidine deaminase domain; and
(ii) a non-protein uracil-DNA glycosylase inhibitor (npUGI), wherein the npUGI is a small molecule inhibitor of UDG.

2. The base-editing system of claim 1, wherein the DNA-binding domain is selected from the group consisting of a Cas domain, a Transcription Activator-Like Effector (TALE) domain, and a Zinc finger (ZF) domain.

3. The base-editing system of claim 2, wherein the Cas domain is selected from the group consisting of a Cas 9 domain, a Cas12a domain, a Cas12h domain, a Cas12i domain, a CasX domain, a CasY domain, a C2c1 domain, a C2c2 domain, and a C2c3 domain.

4. The base-editing system of claim 3, wherein the Cas domain is a Cas12a domain.

5. The base-editing system of claim 4, wherein the Cas12a domain comprises an amino acid sequence with at least 85% sequence identity, at least 90% sequence identity, at least 95% sequence identity, or at least 99% sequence identity to SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413.

6. The base-editing system of claim 4, wherein the Cas12a domain is SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413.

7. The base-editing system of claim 3, wherein the Cas domain is a Cas9 domain.

8. The base-editing system of claim 7, wherein the Cas9 domain comprises an amino acid sequence with at least 85% sequence identity, at least 90% sequence identity, at least 95% sequence identity, or at least 99% sequence identity to SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 325, SEQ ID NO: 674, SEQ ID NO: 1869, SEQ ID NO: 1870, SEQ ID NO: 1871, SEQ ID NO: 1872, SEQ ID NO: 1873, SEQ ID NO: 1874, SEQ ID NO: 1875, or SEQ ID NO: 1876.

9. The base-editing system of claim 7, wherein the Cas9 domain comprises SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 325, SEQ ID NO: 674, SEQ ID NO: 1869, SEQ ID NO: 1870, SEQ ID NO: 1871, SEQ ID NO: 1872, SEQ ID NO: 1873, SEQ ID NO: 1874, SEQ ID NO: 1875, or SEQ ID NO: 1876.

10. The base-editing system of any one of claims 3-9, wherein the Cas domain is selected from the group consisting of a Cas nickase domain and a deactivated Cas domain.

11. The base-editing system of any one of claims 1-10, wherein the base-editing system further comprises a guide RNA, wherein the guide RNA has a length in the range of 15-100 nucleotides, and wherein the guide RNA comprises a sequence of at least 10 contiguous nucleotides that is complementary to a target sequence.

12. The base-editing system of claim 1 or claim 2, wherein the DNA-binding domain is a TALE domain.

13. The base-editing system of claim 1 or claim 2 wherein the DNA-binding domain is a ZF domain.

14. The base-editing system of any one of claims 1-13, wherein the cytidine deaminase domain is an apolipoprotein B mRNA-editing complex (APOBEC) family deaminase domain.

15. The base-editing system of claim 14, wherein the APOBEC family deaminase domain is selected from the group consisting of an APOBEC1 deaminase domain, an APOBEC2 deaminase domain, an APOBEC3A deaminase domain, an APOBEC3B deaminase domain, an APOBEC3C deaminase domain, an APOBEC3D deaminase domain, an APOBEC3F deaminase domain, an APOBEC3G deaminase domain, an APOBEC3H deaminase domain, and an APOBEC4 deaminase domain.

16. The base-editing system of any one of claims 14-15, wherein the APOBEC family deaminase domain comprises an amino acid sequence with at least 85% sequence identity, at least 90% sequence identity, at least 95% sequence identity, or at least 99% sequence identity to SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894.

17. The base-editing system of any one of claims 14-15, wherein the APOBEC family deaminase domain comprises SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 607, SEQ ID NO: 608, SEQ ID NO: 609, SEQ ID NO: 610, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, or SEQ ID NO: 1894.

18. The base-editing system of any one of claims 14-17, wherein the APOBEC family deaminase domain is an activation-induced deaminase (AID) domain.

19. The base-editing system of any one of claims 1-18, wherein the small molecule inhibitor of UDG is a compound of formula (I): 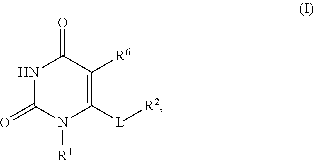
or a pharmaceutically acceptable salt thereof, wherein:
R¹ is H, a furanose carbohydrate, a pyranose carbohydrate, a carbohydrate mimetic, C₁₋₁₆alkyl, C₁₋₁₆alkenyl, C₁₋₁₆alkynyl, C₁₋₁₆alkoxy, or C₆₋₂₀aryl, wherein the furanose carbohydrate, or a derivative thereof, pyranose carbohydrate, or a derivative thereof, carbohydrate mimetic, C₁₋₁₆alkyl, C₁₋₁₆alkenyl, C₁₋₁₆alkynyl, C₁₋₁₆alkoxy, or C₆₋₂₀aryl is independently optionally substituted with one or more substituents selected from the group consisting of hydroxyl, halo, cyano, NO₂, N(R⁴)(R⁵), C₁₋₁₆alkoxy, and C₆₋₂₀aryl, wherein the C₆₋₂₀aryl is further optionally substituted with one or more substituents selected from the group consisting of C₁₋₁₆alkyl, C₁₋₁₆alkenyl, C₁₋₁₆alkynyl, C₁₋₁₆alkoxy, hydroxyl, halo, cyano, NO₂, and N(R⁴)(R⁵);
L is O, S, or *—N(R³)—**, wherein R³ is H or C₁₋₁₆alkyl, and ** indicates the point of attachment to the R² moiety and * indicates the point of attachment to the remainder of the molecule;
R² is H, N(R⁴)(R⁵), or C₆₋₂₀aryl, wherein the C₆₋₂₀aryl is independently substituted with one or more substituents selected from the group consisting of C₁₋₁₆alkyl, C₁₋₁₆alkenyl, C₁₋₁₆alkynyl, and C₁₋₁₆alkoxy; and
R⁴ and R⁵ are each independently H or C₆₋₂₀aryl; and
R⁶ is H or halo.

20. The base-editing system of claim 19, wherein R¹ is H.

21. The base-editing system of claim 19, wherein R¹ is C₁₋₁₆alkyl, wherein the C₁₋₁₆alkyl is optionally substituted with one or more substituents selected from the group consisting of hydroxyl, halo, cyano, NO₂, N(R⁴)(R⁵), C₁₋₁₆alkoxy, and C₆₋₂₀aryl, wherein the C₆₋₂₀aryl is further optionally substituted with one or more substituents selected from the group consisting of C₁₋₁₆alkyl, C₁₋₁₆alkenyl, C₁₋₁₆alkynyl, C₁₋₁₆alkoxy, hydroxyl, halo, cyano, NO₂, and N(R⁴)(R⁵), wherein R₄ and R₅ are each independently H or C₆₋₂₀aryl.

22. The base-editing system of any one of claims 19-21, wherein L is NR³, wherein R³ is H.

23. The base-editing system of any one of claims 19-21, wherein L is O.

24. The base-editing system of any one of claims 19-23, wherein R² is C₆₋₂₀aryl, wherein the C₆₋₂₀aryl is independently substituted with one or more substituents selected from the group consisting of C₁₋₁₆alkyl, C₁₋₁₆alkenyl, C₁₋₁₆alkynyl, and C₁₋₁₆alkoxy.

25. The base-editing system of any one of claims 19-24, wherein R⁶ is H.

26. The base-editing system of any one of claims 19-24, wherein R⁶ is halo.

27. The base-editing system of any one of claims 19-26, further comprising a pharmaceutically acceptable carrier, diluent, or excipient.

28. The base-editing system of any one of claims 19-27, wherein the compound of formula (I) is incorporated into a macromolecule.

29. The base-editing system of claim 28, wherein the macromolecule is an oligonucleotide.

30. The base-editing system of any one of claims 1-29, wherein the npUGI reversibly inhibits an activity of an UDG.

31. The base-editing system of any one of claims 1-30, wherein the UDG is an animal UDG.

32. The base-editing system of any one of claims 1-30, wherein the UDG is a plant UDG.

33. The base-editing system of claim 32, wherein the plant UDG comprises an amino acid sequence with at least 85% sequence identity, at least 90% sequence identity, at least 95% sequence identity, or at least 99% sequence identity to SEQ ID NO: 373, SEQ ID NO: 374, SEQ ID NO: 375, SEQ ID NO: 376, SEQ ID NO: 377, SEQ ID NO: 378, or SEQ ID NO: 379.

34. The base-editing system of any one of claims 32-33, wherein the plant UDG comprises SEQ ID NO: 373, SEQ ID NO: 374, SEQ ID NO: 375, SEQ ID NO: 376, SEQ ID NO: 377, SEQ ID NO: 378, or SEQ ID NO: 379.

35. The base-editing system of any one of claims 1-34, wherein the fusion protein and the npUGI are chemically linked.

36. The base-editing system of any one of claims 1-34, wherein the fusion protein and the npUGI are not chemically linked.

37. A base-editing system comprising (i) a fusion protein comprising (a) a Cas9 nickase domain or a dCas9 domain and (b) an APOBEC family deaminase domain; and (ii) a npUGI.

38. A base-editing system comprising (i) a fusion protein comprising (a) a Cas12a nickase domain or a dCas12a domain and (b) an APOBEC family deaminase domain; and (ii) a npUGI.

39. A base-editing system comprising (i) a fusion protein comprising (a) a TALE domain and (b) an APOBEC family deaminase domain; and (ii) a npUGI.

40. A base-editing system comprising (i) a fusion protein comprising (a) a ZF domain and (b) an APOBEC family deaminase domain; and (ii) a npUGI.

41. The base-editing system of any one of claims 37-40, wherein the npUGI is selected from the group consisting of a small molecule inhibitor of uracil-DNA glycosylase (UDG) and a nucleic acid inhibitor of UDG.

42. The base-editing system of any one of claims 37-38 and 41, wherein the npUGI is a RNA guide of the Cas9 nickase domain, the dCas9 domain, the Cas12a nickase domain, or the dCas12a domain, wherein the RNA guide comprises a 5′ end and a 3′ end, and wherein the RNA guide comprises one or more deoxyuracil (dU) bases added to the 5′ end or the 3′ end.

43. A method of editing a target nucleic acid molecule, comprising contacting a target nucleic acid molecule with the base-editing system of claim 3, wherein the DNA binding domain of the fusion protein is the Cas domain.

44. A method of editing a target nucleic acid molecule, comprising contacting a target nucleic acid molecule with the base-editing system of claim 2, wherein the DNA binding domain of the fusion protein is the TALE domain or the ZF domain.

45. The method of claim 43 or claim 44, wherein the fusion protein and the npUGI of the base-editing system are chemically linked.

46. The method of any one of claims 43-45, wherein the fusion protein and the npUGI are chemically linked by in vitro complexing.

47. The method of claim 43 or claim 44, wherein the fusion protein and the npUGI of the base-editing system are not chemically linked.

48. The method of any one of claims 43-47, wherein the fusion protein and the npUGI are co-delivered, wherein the fusion protein is delivered before the npUGI, or wherein the npUGI is delivered before the fusion protein.

49. The method of any one of claims 43-48, wherein the target nucleic acid sequence is a double-stranded DNA (dsDNA) sequence, and wherein the target nucleic acid sequence is in the genome of an organism.

50. The method of any one of claims 43-49, wherein the target nucleic acid sequence is associated with a disease or disorder, and wherein the target nucleic acid sequence comprises a point mutation.

51. The method of claim 50, wherein the point mutation is a T to C point mutation, and wherein the T to C point mutation is associated with the disease or disorder.

52. The method of claim 50 or claim 51, wherein the point mutation is a T to C point mutation in a codon of the target nucleic acid sequence, and wherein the point mutation results in an amino acid change in a polypeptide encoded by the target nucleic acid sequence as compared to a wild-type polypeptide encoded by a wild-type nucleic acid sequence without the point mutation.

53. The method of any one of claims 50-52, wherein contacting the target nucleic acid sequence with the base-editing system produces a nucleic acid sequence that is not associated with the disease or disorder.

54. The method of any one of claims 50-53, wherein contacting the target nucleic acid sequence with the base-editing system produces a wild-type nucleic acid sequence without the point mutation.

55. The method of any one of claims 50-54, wherein the disease or disorder is cystic fibrosis, phenylketonuria, epidermolytic hyperkeratosis (EHK), Charcot-Marie-Toot disease type 4J, neuroblastoma (NB), von Willebrand disease (vWD), myotonia congenital, hereditary renal amyloidosis, dilated cardiomyopathy (DCM), hereditary lymphedema, familial Alzheimer's disease, HIV, Prion disease, chronic infantile neurologic cutaneous articular syndrome (CINCA), desmin-related myopathy (DRM), a neoplastic disease associated with a mutant PI3KCA protein, a mutant CTNNBl protein, a mutant HRAS protein, or a mutant p53 protein.

56. The method of any one of claims 43-55, wherein the contacting is performed in vivo.

57. The method of any one of claims 43-55, wherein the contacting is performed in vitro.

58. The method of any one of claims 49-57, wherein the organism is a mammal, and wherein the mammal is selected from the group consisting of mouse, rat, human, pig, cow, chicken, rhesus monkey, and guinea pig.

59. The method of any one of claims 49-50, wherein the target nucleic acid sequence is selected from the group consisting of a target nucleic acid sequence associated with a disease, a target nucleic acid sequence associated with a disorder, a target nucleic acid sequence associated with metabolic function, a target nucleic acid sequence associated with reproductive function, a target nucleic acid sequence associated with disease resistance function, a target nucleic acid sequence associated with stress tolerance function, a target nucleic acid sequence associated with agronomic function, and a target nucleic acid sequence associated with nutritional function.

60. The method of any one of claims 49-51, wherein contacting the target nucleic acid sequence with the base-editing system produces a nucleic acid sequence selected from the group consisting of a nucleic acid sequence with a corrected deleterious mutation, a nucleic acid sequence derivative, a modified nucleic acid sequence, a nucleic acid sequence with an inserted sequence, a nucleic acid sequence with improved function, and a nucleic acid sequence with altered function.

61. The method of any one of claims 49-52, wherein the target nucleic acid sequence comprises a point mutation, and wherein contacting the target nucleic acid sequence with the base-editing system produces a wild-type nucleic acid sequence without the point mutation.

62. The method of any one of claims 59-61, wherein the contacting is performed in vivo.

63. The method of any one of claims 59-61, wherein the contacting is performed in vitro.

64. The method of any one of claims 59-63, wherein the organism is a plant, and wherein the plant is selected from the group consisting of sorghum, corn, tomato, rice, soybean, and wheat.

65. A method for in vivo editing of a cytosine residue of a target DNA sequence, the method comprising
a. contacting the target DNA sequence with the base-editing system of any one of claims 3-11 and 37-38, wherein the DNA binding domain of the fusion protein is the Cas domain, and a guide nucleic acid; and
b. cultivating the cell through a cell division.

66. A method for editing a nucleobase pair of a double-stranded DNA sequence, the method comprising:
a. contacting a target region of the double-stranded DNA sequence with the base-editing system of any one of claims 3-11 and 37-38, wherein the DNA binding domain of the fusion protein is the Cas domain, and a guide nucleic acid, wherein the target region comprises a target nucleobase pair;
b. inducing strand separation of said target region;
c. converting a first nucleobase of said target nucleobase pair in a single strand of the target region to a second nucleobase; and
d. cutting no more than one strand of said target region;
wherein a third nucleobase complementary to the first nucleobase base is replaced by a fourth nucleobase complementary to the second nucleobase and the method causes less than 20% indel formation in the double-stranded DNA sequence.

67. The method of claim 65 or claim 66, wherein the cut single strand is hybridized to the guide nucleic acid.

68. The method of any one of claims 65-67, wherein the cut single strand is opposite to the strand comprising the first nucleobase.

69. The method of any one of claims 66-68, wherein the first base is a cytosine.

70. The method of any one of claims 66-69, wherein the second base is an uracil.

71. The method of any one of claims 66-70, wherein the Cas domain is selected from the group consisting of a Cas nickase domain and a deactivated Cas domain.

72. A method for editing a nucleobase pair of a double-stranded DNA sequence, the method comprising:
a. contacting a target region of the double-stranded DNA sequence with the base-editing system of claim 3, wherein the DNA binding domain of the fusion protein is the Cas domain, and a guide nucleic acid, wherein the target region comprises a target nucleobase pair;
b. inducing strand separation of said target region;
c. converting a first nucleobase of said target nucleobase pair in a single strand of the target region to a second nucleobase;
d. cutting no more than one strand of said target region; wherein a third nucleobase complementary to the first nucleobase base is replaced by a fourth nucleobase complementary to the second nucleobase; and
e. replacing the second nucleobase with a fifth nucleobase that is complementary to the fourth nucleobase, thereby generating an intended edited basepair, wherein the efficiency of generating the intended edited basepair is at least 5%.

73. The method of claim 72, wherein the cut single strand is hybridized to the guide nucleic acid.

74. The method of claim 72 or claim 73, wherein the cut single strand is opposite to the strand comprising the first nucleobase.

75. The method of any one of claims 72-74, wherein the first base is a cytosine.

76. The method of any one of claims 72-75, wherein the second base is an uracil.

77. The method of any one of claims 72-76, wherein the Cas domain is selected from the group consisting of a Cas nickase domain and a deactivated Cas domain.

78. The method of any one of claims 72-77, wherein the Cas domain is selected from the group consisting of a Cas 9 domain, a Cas12a domain, a Cas12h domain, a Cas12i domain, a CasX domain, a CasY domain, a C2c1 domain, a C2c2 domain, and a C2c3 domain

79. The method of any one of claims 72-78, wherein the Cas domain is the Cas12a domain, and wherein the Cas12a domain comprises an amino acid sequence with at least 85% sequence identity, at least 90% sequence identity, at least 95% sequence identity, or at least 99% sequence identity to SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, or SEQ ID NO: 413.

80. The method of any one of claims 72-79, wherein the Cas domain is the Cas9 domain, and wherein the Cas9 domain comprises an amino acid sequence with at least 85% sequence identity, at least 90% sequence identity, at least 95% sequence identity, or at least 99% sequence identity to SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 325, SEQ ID NO: 674, SEQ ID NO: 1869, SEQ ID NO: 1870, SEQ ID NO: 1871, SEQ ID NO: 1872, SEQ ID NO: 1873, SEQ ID NO: 1874, SEQ ID NO: 1875, or SEQ ID NO: 1876.

81. A method for in vivo editing of a cytosine residue of a target DNA sequence, the method comprising
a. contacting the target DNA sequence with the base-editing system of any one of claims 12-13 and 39-40, wherein the DNA binding domain of the fusion protein is the TALE domain or the ZF domain; and
b. cultivating the cell through a cell division.

82. The method of any one of claims 65-81, wherein the fusion protein and the npUGI are chemically linked.

83. The method of claim 82, wherein the fusion protein and the npUGI are chemically linked by in vitro complexing or in vivo complexing.

84. The method of any one of claims 65-82, wherein the fusion protein and the npUGI are not chemically linked.

85. The method of any one of claims 65-84, wherein the fusion protein and the npUGI are co-delivered, wherein the fusion protein is delivered before the npUGI, or wherein the npUGI is delivered before the fusion protein.

86. The method of any one of claims 65-85, wherein the DNA sequence is in the genome of an organism.

87. The method of any one of claim 86, wherein the organism is a mammal selected from the group consisting of mouse, rat, human, pig, cow, chicken, rhesus monkey, and guinea pig.

88. The method of any one of claim 86, wherein the organism is a plant selected from the group consisting of sorghum, corn, tomato, rice, soybean, and wheat.
